SEQUENCE LISTING (25 sequences)

SEQ ID NO: 1 - DNA, 43120 bp, Homo sapiens
agagtgcagcccccagctatttttctccgtggcggcggcgacgagcggaagttcttgggagcgccagttccgtctgtgtgttcgagtggacaaaatggcgaagatcgccaagactcacgaaggtaagcggtctttccctgcttacgtgttttcttcgttgctagcctaataaaagccttttttcctaggattctgcctttttctatggtttctttcgtaggagctgagcccgcgtttcggggccaagggtcgtctcctccgcgccagcggcgcaggccactgccccgcgtaatggcgtcctcctcttacacccccgacaggcgctttgttcagtgtttgggacctgctacttcagagtagcttctcttctctgaccagagggcctcgtttaagcggcttcctcagtaggattcgtgggcaagggatcggggaagcgggcgtggggagaaggtctcactcgcctctcgacactgtcgtccccatagtttgtgcagaagagggtcagaaaagcactgaaatacggcagccgcttttccggcagactgaatcgaatttggttaaaccgagattccgttactcctacccttgtgtgtcttcaagcgtcagttacttgtttatttttcttgcacgaaacgatttggggataaaatctggtagttgttttccctcccttagtcacttttagtataatattgatctttttctttggagactggttattggaggagcagatggaagaggaaaacataaattactttcattaatggccgaagctttcaagctagttgatcgttttatggaatacagagtgtgcaaattaggaaaacgttgggaatagactggagttcattaggaagtatccagagatgacagaaaatgggatcacttttcagaaaagacgaggcatgagaagttggaactgcattctttgatttctgacttggaatccgagattttcactgagaacgagacttctaagtcacttttgtttcatatgttctttctgggtatttggctcggtattagtctgtgagaggtaagagactgaggccatccaaaattttacagtctgataggagaaacagatgtgtatgttctcattctctcatgaggccagatgcatgcttaagtagaagaaaaagcagcatgttctgggaacctgggtaacggaatgattatgtttagggttgtttcactgaggaggttacatgaccgtgttttaaaaggatcagtcagttttaaatcgggaaggacatctgagcctgagaacatcttgtaacaaggtattgaatcgtgaaaggtgtgtttgagtagtagtccactgtagcaggaacagagggtgcaagtagagaagaaataaatgaatctggaaaggttggattaggaccagaatgtgaatggctttgaatagttgggtcacccagaggacctgaagtccagctttctgctatttatcaaacatgctaatgaattgtcaatgactttaatattagtataccattttaggtatttaggatttaaatgctctcttagtattcctaggctttattattttgtctttagactatctcaaatataaccttttgcaaagtaagtagaaaatataaagttgtgctgtttttgttcattcaggatgttattcatttggggatattgatagtatagctttgatacttatttattggtaacttcagtataagatgccctcagacaggaaaatcagaattctgatcagttttcctgaattttttagaaacccagctaaaaactttggtctattgctattcattggccaaacttgttatttttgagattattctatcaatgtgaattaagtgataccataagaaaatagcagaacattcaatacttgagcactgtctgtgcccatccctatgggatatgtcaggggatgataaatgtttagtagaaatacacataatactgacatagcttgttgcttccttcctaatggaactctagttcttcatggtccatcactgttttctcttgttaagccatttttgataaactgaagcagaattaatgctttttggggcagtgctgctatagtactcagtataaatgttatattgcttagaatagtctgttgtgttaagattttctcttctccttagctccaagattgagataaactgataactatttatttattttgctgtcatattcctttcaaacatttctttttttattttttatttttttttgagatagggtctttttctgttgcccaggcagaagtgcagtggtgcaatctcggctcacagcaacctccgccttttaggttcaagcgattgtcctgcttcagcctcctgagtactagtatcgcaggcatgtgccaccacacccgactaatttttgtatttttagagacagggtttcgccatgttggccaggctggtctcaaactcctgaccacaagtgatcctcccaccttggcctcccaaagtgctgggattactggcatgagccaccatgcctgaaccctttcaaacatttctattaggatcaggcctcacatcctctttaaccaatctgattatattttaccctggccaatgtggcttatgctttccaggattgaaatataaaaagaactggaattactcaaatcagataaaatcttttagatctttccacgtattatgtcaggtgtatggttatgaatatgcataatcttgccatccagcagttcttcagatactgctgactttggcatacaaacagggaacacacattattctctgttttgtaaggggaaaatggtttaaccaaaaatcctacatatcagcttgttttgccactaatcctttgaattagattttttgggacatcacaaagctgaaaaagttttttccctaattctttgcttgataaatggctggaatagttatagttttgttattgtgtatcttgctgatctatatattttttcctgtgtttatttttgagaccgggtcttgccctgttgcccaggttggagtgcagtggcgtgatcatagctcacttaacctacaactgctgggctcaagtgatcctcccatttcagcctcctgagtagctacaactacaggcgtgtgctaccatgcctggctaatttttacaatttttttttagagatggggtctcgctatgttacccaggctgatctcgaactcctgggctcaagcagtcctgccttggcctcccaccgtgctaggattacaggtgtgagccacgatgcctggccttgaaattttttttaatagaattaatcatttaggaatcaatttatcagtattgtttgtagtgttcagtaaaatgatttatattatagttagttgtcctattggagttttgtttaatgaaaaagctgagggttgggattcagaatatacttcctgtttttctgtgatgtcttttagaagccttgtattttggaaatagttgttcaccggttatatctggctgaaggagagtagatatcacttagggaccagactgaaaggtgtaggtgagacattaacatctgagggcagtatctgtgtaacatgtaatgagcagtgattagaacactgaaaataattcagacgaaaaatgaaaaataaaaatcaaggaggaagaagtgctacaagttgggtttagcaggatctaaggcttagaaagaagaaaagggctgggcatggtgtctcacacctataatcccagcactttgggaggctgaggtgggcagatcacccaaggttaggagttccagaccagcctggccaacgtggtgaaaccccctctctactaaaagtacaaaaaaattaaccgggcatggtggcgtgagcctgtgatcccagctcctcgggaggctgttgcaggagaatcgcttgaacccaggaggcgaaggttgcagtgagctgagatcacgccacgccactgcactccagcctgggcgactgagtgaaactctgtctcaaaaaaaaaaaaaaaaaaaaaaaaaagaagaaaaggcacaggaggggaaatcagagggacagaattggtggggagcagttaaatagttttatcagatgtgaccttctgctatcagtgatatgaattgtttttctcaagagcttgatatgtaggtctgagttttaaaatacaggaggccaggggttgctcacgcatgtaatcccagcattttgggaagtcaagacaggaggattgcttgagactgcaaacatacagaaaagtagagagcggctggcgctatactccagcctgggcaacagagcaggactctgtctcagaaaaacaaaacaacaggccgggcacagtgtgtgaagacaggttttaataacactagggtaggctgggcacggtggcttacgcctgtaatcccagcactttgggaggctgatgtggatggatcaccagaggtcaggagtttgagaccagcttgatcaacatggtgaaaccctatctctactaaaaataaaaaaattagctgggtttggtggtgcgtgcctgtaatcccacctgcttgggagcctcaggcaggagaatctcttgaaccaggaggtggaggttgcagtgagccgagatcgtgccactgcacttcagtttgggcaacagagtgagactttgtctcaaaacaaaataacaaaaaaaactaccgtgagtagtaaaatgaacccctcatatactcatcacctagcttcaacagttacaagcttaggccagctttgtttaatttgtacactggtcacttctactcttcccattattttgaagcagatcttagacataccactttatccataaatatttcagtatgtatttctgtgtagggacttaaaaagataataccatgattacacctaaaattaaaataataaaatatctagtcactgttctgttttacccattcttttaaactttgtttttcagtttcagtcaggcttccaagaaaactgataacattttgattggttggtttctcttatatctttttcatttcacagtacattgtgatttaatgatttatctcttaaatctttttcagttcataggttcctcctgtctctttctttttccttgcacttcatttgttgcagaacctttgtcctgtggtttcgcacagtctggaggaattggggaaagtggctgtctaaactggttccctccttcccattactgctgcaatcaagatgttagggggcctacaagcaatggaagcacccagaaaattattggctctaagtatgagcatagttctgaaatcatacttcaggtttagattgtgctagtcatgggaaggaaatttttttttttttttttgagacagagtcttgctctgttgtccaggctggagtgcagtcgcacaatgtcggctcactgcaagctccgcctcccgggttcaagcgatacttctgcctcagcctcccgagtagctgggactacagacgcgcgccatcacacccagctaatttttgtatttttagtagagatggggtttcaccacattggccagactggtctcgaactcctgacctcaggtgatccatccacatcggcctcccaaagtgctgggattacaggcgtgagccaccgtgccggcctgccttagtgttattaaaacctgtcttgacacaaaaacctctaaacaaatgtttacagtagccttacttgtaatcactgcaaacagctcgatgtacttcagctagagaatggataaacacactgtagtacaatggaatactacccagtaataaaaaggcagaaactattgatgtgcacaacagtgtggaaattggaatatttaagggtttaaatgggttaaagagtgccagcttttggcagttttgtgtggccaataggaagtcctagtctcttcttgctcagtcactagcagacagtatcccttttttctttgtcttcacatctgaatgtattgaactatgagtccaaactgggaagggattaggctggaacaggatttgttcattttggtcataattgaagttggaagagattattagcatagacgtttctttgcctcaagtcttcattgtttttgacctctggttactcattttggtatgttctctcattttttattttcatagttgccaacccccgccttttttttttgagatggagtttcactgtgtcgtccgggctggagtgcagtggcgtgatcttggctcactgcaacctctgcctcccagactcaagcgattctcttgttgcccaggccagagtgcagtgctgtgatctcggctcactgcaacctccacctcccaggttcaagcgattctcatgcctcagccttccaagtagctgggattacaggcgtgccaccaccatgtctggctaatttttgtatttttagtgaagacagggtttcaccatgttggccaggccggctttgaactcctgatctcaagtgattcaactgtcttgacttcccaaagtgctgggattacaggcgtgagccaccgcgctcagccagttgccaagcagttttatgcattgatcatttgttgtcagaaatgatctatttgcatatttttagtcagtttatatttaagaaattataggaatacttacacaccaggctgtaacttagtgttggttttgaaaatacggagttagagacaacattgttattcccagagataaaaaatttgaagtggaattttacaaaccttggctggcatggataggggagagaaaaatttggaaaattacttttaattacttctacagattacctatttatttatttacttacttatttagaggcaaggcctggctctaacacccaggctagagtgcagtggtgtgatctcagctcactgcaacctccgcctcctgggctcaagccatcctctgacctcagcctcctgagtatctgggactacaggtgtgcacctgtagtgactaatttttgtatttttgtctaatttttgtagagacagggtttcatcatgttgcttaggctggccttgaacttgtgaactcaagtgatctgcctgcctgggcatcccaaagtgcccagcctcattaccttttaattaattaattaattaattaattaatttgagacggagtcttgctctgtcgcctaggttggagtgcagtggtgcgatctcggctcactgcatcctctgcctcttgggttcaagcaattctctgactcagcctcctgagtagctgggattccaggcacccaccaccacacctggctaatttttatatttttagtagagatggggtttcaccatcttggccaggctgatcctgaactcctgacctcgtgatccacccgcctcggcctcccaaagtgctgggattacaggcgtgagccaccatgcccggcccagattaccttttatgaaatattttaacatgcagaagagaataggacaaaatataatgaacactaacccactgctcagctttatcagttcttcattttctgccgtattttttcccacctttttatttattttgaaaacgaaacctcagacacaaagttaccttatttatgaatgtcatggtaataatttgtttgctcataaatatttgggtatgttatatttggtattttagcacatagtttattaagaaatttctgagtttttagttctctgaattgcataataagctgctgagttttatggaatctgacggcttaattttatggatcacatggtgtatgtgtaaatgtgctgcagtttgcagtgtgttaacaaagcccattaagctgtttggactttatatctatagtttccagcagacagaaaacaatgcctgatttgcatataaacatttaattgtactgcctataagaagttcagtaatatactcaccttaggattaattaactacaaacagctgcctagccccaggagattttgggctgggtaattttgttactaaattttaataagaacctagtcatgatttacatagatttcataccaacactatttcagtactttatgaacctttgtaattggaaaactctgatgctaatcagcaggtacataaaatcaatagttaaaattcagtagctgaaattagttccagaataagtgttatccaaatgagaaactagttgctgaaacttcaggatggaaaagttgtttagatggaaaataagagagatgcctgataagggggaaaaagcaagttgcacaatatgtgtagtgtgcagtttttataaaacagcacccctttcctgttttttagttgtgtatgaggtagagaaaagtataaaatgatgctctaaattggaaaatttggtaggggtgggctggggttggaaatgggaggggtgaagggccaggtaaaatagtttcctagggtgtttgttaatgatgcagaatcctgggcttcacagcctgagttggagtttcagagttagtgtatccaagacctaaggttctacctttgtaacaggtgcaccaggtcatttctttgtaggtggtccttatactagactaatgctgtagaggagattgcagactatttctcctgattcatccatttgtttcttacctaaatcctcatggaagtagcatttgtggtccttttacaaaaacctatttaggtaaaagtgataaatgcacctgtggtttaaaaaaaaagcctgaagctgggcgtggtggtgggcatctataatcccaactatttgggaggctgaggcaggagaatggcttgaacctgggaggcggaggttgcagtgagccgagatccagccactgcactccagcctgggcaacaagagcaaaactccgcctcaaaaaaagaaaagaaaagaagatgcccgaagggggtgggagtggggcaaatgtagattaaatttttcaaaatatgaaactacaaacagaaacaaaagtagagatcatagtaaaaagaaccaccctgctcatgtgaggatatttctggctgcctgggctgctataatggtttggggacaaagtaacatctgtgcatgtagtggtgctgtgtaacacaacacactataataattttgagatccgatcacaactggggctgctaaactcagcattaaaactattctatcattttgagtttctttctttctgtttgtttctgagacagtctctctctcatccaggctggagtgcagtggcagcaatcgtggcttactgcagccttgacctcctgggctcaaacagtcctcccacctgagccccctaagtggcaggggactataggtttgccaccatacctggctaattttagtattttttgtagagatagggttttgccatgtcgcccaggctggttttgaactcctgggctcaagtgatccgcccacctcagccttccaaagtgtttggatgacatgcgtgagctaccgcgtctgcctccagtttgtgagacgtgctcctgttcctttcttatgtggaaatgtgacccagccgtgccttagactagataaggacattttgttatcctgcacatctatcttgaggaactatagagggctttctcccttagctgggggggtgggaggttttgcaaatacaggatgctatttgaatctttatcttttgattcatgcagctagcaattgagcttttttattttttatttgatttatttatctatttattttttgagacggagtttcgctcttgtcgcccaggctggagtgcaatggcgtgatctcgacttactgcaacctccaccttccgggttcatgtgattctcctgcctcagcctcccgagtagctgggattacaggcgcctgccaccatgcccggctaatttttgtatttttagtagagacggggttttagcacattggccaggctggtcttgaactcctgacctcaggtgatctgcccacctcggcctcccgaagtgctgggattgcaggtgtgagccactgcgcccggcctaatttttattcatttatttatttatttgagatgagttttgctctttgcccaggctaaagtgcagtggcacgtgatctcggctcactgcagccttggcctcccgggttcaagcagttctgcctcagcctcctgagtagctgggactagaggcgtgcgccattacacctggctaactttgtatttttactggagaggaggtttcaccatcttggctaggctggtctcgaatgcctgacttcaggtgatccgcccgccttggcctcccaaagtgctgggattataggcgtggaccaccgcgcccggccagcaagtaagctttttaaaagtgcctggtgtcaaagggcttaattcagcatgggctggcaagaatttaaacagctatttgtcaggccatggaaatcttttgcaatcgaaactatttctttatacatctatgtattatttagattattaaaacaaaacattactttgcaaaattaacctggtgtcaggaaagactacaaaatggaagactagtctgtatttaaagaagagcaaagagtgacaactaaatgcagtgtatggtgcttgattggatcatgggttaaaaaagaatctatagaagctgttactagggcaactggggacattttaagctaggttatatgtttgttagatgatattttgtcacggtttttttttttttttttgagagatagagtcttgctctgtcacccaggttggagtgcagtggtgtgatctgcaacctccgcctcccgggttaaagtgattctcctgcctcaacttcccaaatacctgggactacaggtgcatgcccccacgcccagctactttttgtattttttagtagagacggggtttcgctatatgttggccaggttggtctcgaactcctgacctcaagtgatcgcccacctcctcctcccaaagtgttgggattataggcgtgagccacagcacccagatctgcccacctcatcctcccaaagtgttgggattgtaggcgtgagccacatgcccagcctatcatggtttaatttctagagcttgcttacgctgttgtgattactagtctcttatatggtgcaattattgtacattttctgtatttaagagagtattttgataattacctagtaaagaaatgcattgtgttgggagttcttttggaaagtgaaattaagaaagtctgtgttatctttttatagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgtaagttacaaattatgtacaagaggcagttatttttttgagagagcaagaaatatgaataacttttgttgcagccatctggtttaatctggagaaacatttttgggatcttttagaggataaaactactggaggtatcctctctaaggttcagaaacttcactggaaatgctttgttctgagaatttatggtaaagactagtttgtttatccagtgaagcacctgccagccattacctttataaagttaagagtaaagtaaatccctaccattacttatcagctctatctagagtagcctgtgtgagaagtgacttaattgtaaatagccatctgtccttgtgatttgattactgacttgtacttgatactgtttttctcctcttggtatcttaatgtgaatggattttcttttcttatctttttttttttttttttgagacagtctcacctgtcatccaggcttgagtgcagtggactgcaacctccgcctcccggtttcaagtgattctcctgcctcagcctcccaaatagctgggattacaggcgcctgctaccatgcctggctaatttttgtatttttagtagagatggggttttgccatgttggccaggctggtctggaactcatgccttcaggtgatccacctgccttggcctcccaaagtgctgcgattacaggcttgagccactgcgcccagcctggaatttttcttagtactgatattgatgagcttttcatgtctgtttgaattcctatggaggaggactgcctttcattttgaaatctgttccctcaaagcattttttatgtattccaggtagcttatattttatcagaccattccaaactccaaatttggtgattcagttacagataaattttattttatgtatatagattatcaaactggtttttttttttcgtctcactctgttgcccaggctggaatgcagtggtgcaatcttggcttactgcatgctccgcctccagggttcatagcattctcctgcctcagcctcccgagtagctgggactacagatgcctgccaccaagtccggctaattttttgtatttttagtagagacggggtttcaccgtgttagccaggatggtctcgatctcctgagctcgtgatccacctgcctcagcctcccagagtgctgggattacaggcgtgagccaccgcgcctggttattttttatttttttttaatttttggataggtgatgtatatttctttttttattattattatactttaaattctagggtccatgtgcacaatgtgcaggttacgtatgtatacatgtgccatactggtgtgctgcacccattaacttgtcatttacgttaggtatatctcccaatgctatccctcccccctacccccaccccatgacaagccccggtgtgtgatgttccccatcctgtgtccaggtgttcttattgttcagttcccacctaggagtgagaacatgcggtgccaactggttatttttaagaaattcaagtcacattttatagttggctctcttcctcttttttttttcttgagagaaatttaaaaatcccaaaaggtgaaatttctagaagccagtcatggactagttagcactttatttgggggtctcataccctaattgtaaaagaaagtgatagctataggtttcagaagctgagggggaaattgtcactttatctttaaataaaataagtgaggtgttaagatctggagaattaccccgtgttgcactttttattttttatttatttttattacaatagagatgagatctcactatgtatgtggttttgttttttggagacaaagtcttgctctgttgcccagaccaaaggcagtggccagatcatgcctcactatagccttcgcctcccgagatcaagcaattttcccacctcagcctcctgagtagctgggactacgtatgcacaccaccacgcctggctaatttttgtattttttttggtagagacagagtttcaccatgttgcccaggctggtcttgaactcctgggctcaattagtcccttgcctcggcctcccaaagtgttgggattatagatgtgagccaccttgcccagctgggtctctgttgcctaggctagtctcaaaactcctacactcaagccattctcccaccttggcctcccaaacagttgtactttttatactagaaaagtaacagtataacattgttctgtttaaacatactactaggggcaaataaatttaaaattaaatatttgtttctatggttgttaaattgtgttaaaaaacatgagttaaaaaaagaaagtagaaagttctaaaataagctgtagttatcaaattgaaaaaacattttgtcttctgtgaaaaaattaaatgcacaggtttttctagtccatacctactttatcttaattgacgtctatagaaaatattccttgcttaatcattttaaaagtaaagataactttactgaattgaatttaatagatttttgaagtgaaagaagaccaattgtgtttctaagttttatgaaggagggcttagacatcacactgtcaatagatcttttgatttcattttgaatctttgcatattctttctaatttttaagttctaacattttgtttatggctttttggggggttttcattgcaggatgacgatgactattcatcatctacgagtttgcttggtcagaagaagccaggatatcatgcccctgtggcattgcttaatgatataccacagtcaacagaacaggtgatctttcattttaaaagcatagtttattcagctaaagaaaagtgaatgtctgagttcccatagaaatccataaattttaggatgctttaaggataattataaataataacttattttgtaaaggtgtaatctttttctttttgacacaaggttttgctgtcttgctcaggctgatcccaaactcctgggctcaaggattcctcctgtctcaacctcccaagtagttgagattatacacacaggctgtcatgcctggctgtaatcatttaatgaagcattgaactatggaaatgtggatatgtatattaccttgtttccatgtgccttatttttttatatttaacatttatctgtgaaacggtacagaatattagtaggcatgtattaaacatttgtgctttatggtgttctgatttttgtttatgatattctgttttgttattttaataaagtggatattaagtgagacatacctcttttcagtatgatccatttgctgagcacagacctccaaagattgcagaccgggaagatgaatacaaaaagcataggcggaccatgataatttccccagagcgtcttgatccttttgcagatggtaattctttcccacttttctataagtattcagaaatttatttgtattaagttgtctttagccctttgattttttttggcatgcttctgaatttgcttttctttttaaaaatcccccttactatagatttgtatagatgtggttgaagtcacagatgccatattcttcatctttataatgcaagttttctttccaggtagaaggacaagaaaaaaatgagccaatgccatagaaacgcaaagggttaaaggttttacttttacttttgttttgggtacgctttctatacttggatcaaatgtatttttctgctcaaaatactttcaaattattgaaagttttcattccaaatcctagatttttaggattgttacccttgagaaactgtttctcacactagagctccccccatagtgctctaaatatagcttagtaaaggcacctactttgctctaagcatacttaaagttaaaacaaaagtatgtatacgaagagtttgatatttttaatagttggagtctgttttgctaattttagtgtgcaagtcagcacaaggggcatagtgagcctgatggaagctttcctcaggtgagtgagtttcacaatcaaaattaggtgcctgtttcttaaaattagattctgctgcaggtttacatactagacttttcagcctgcatgaattagtagggatattctttttttacttgcataattgtaattgatttaaacatgtatgccagaattcacaggcccatactaactgtccttaatttctttcctacagcagtactgctatatgccagtcctgtctgcattcttaagggtgcagttcaacacatcctctctagattatggtgaaaaagtattccaaaggaagtcttatcagagctagtgtcagaaagaatgacactaccaattgggcatgatcttcagcataggaaatgtcttagaattttattattttttcctaaattgtgatgctatactacttgtatagctagttaccacatttctaaagcatagtgtgcctctgtgaatcttgttatatactgtttgggcttctgtaagatattttgaagtttgaaaagcaaatactgaaactttaaccaagagactgacacatctctttgtacaccacgttgcttctgtgtgcatcataataactggtagaagtaatatatttaaagtaatatgtttccagttttaatttatgctttctaagaaataaaaatatgttttctgggtcaacaaaacttaaagcatgaagccctttttagtaacgtgacatcagaataagattataggtatatttttaaaatcagattttctaaactacaattgttttagaacactgtatgaagaacttatctagtcatagttatttgtagtcaggattttaacagcttgcaacaggtaatgttttgaatataaatatctttccaaagtgttactaatggtaaagagataattttctaggcttctattctgctgcttgaagtcagaactgctgatggagacaaaggcacgaaagtgtacgtattccggattagcaacccaggaacccatcacttctgaagactctaaactgtgctgtcattttgtttttatatgcattaaaatatttgttttaaactgctgtagatgtttgtgttcaaacaggttaaattgatcagcaaactcaataaaaagtaagatgtataattattaaagttttccatcaaaaaataatttaaatgattatggaataacataatatttagagcagggcagataaatcagttgaacctgcttattttttatttattgtactctttttcctgttgctgttcttttctgcaggagggaaaacccctgatcctaaaatgaatgctaggacttacatggatgtaatgcgagaacaacacttgactaaagaagaagtatgtaaacctgtctcctgttttaatggttgtgaaagaatataaatacagttgtgattgagagactgttaggtttcttttttgctatttgctgcacaaataatagtattaataaaaattaactgagaaagaatcttacccccaaattataccaccttccaagtaaaacttttcatttctatatccttttctagtcctgggccccttggctgtatatgtttgtatgtatgtgtctcacaagggattttttttttttttttttttttttttttttggacacagtctcactctgtcatccaggctagagtgcagtggcgtgatctcatctcactgcaatctctgcttcccaggttcaagttatcctcccacttcagcctcccgagtagctgggaacacaagtgtgtgtcaccatgcctagctaatttttgtatttttagtagagatggggtttcaccatgttggccaggctggtctcaaactcctgacctcaggtgatctgcctgtctcggcctgccaaagtgctgggattacaggtgtgagccactgtgcctggccattacaggggtggtttctgtgtggagaaggttgatggtgtgtatctgaactcagtatgttagggaaaagtcagagttcagtgtgagattctgtaagtttcggtttggatgtaagtggagaattttgaataattccttttggcctacagagattatgccattttgtctaataactgcctactttttgtttgtttgttttgagtggagtcttcctttgttgcccaggctgaagtgcagtggtgcgatttcagcttaccgcaccctctgcctcctgggtccaagcgattctcctgccacagcctcccgagtttctgggattgcaggtgtgcaccgccatgcctggctaattttcatatttcttttttttttttttttttttagtagaggtggggtttcgttatgttggccaggctggtctcgaactcctgacctcaggtgatctgcccgccttaacctctcaaagtgctgggattacaggtgtgagccaccgtacccagcctcctccattactattatgaatatataatagattttatatgttgaatgagttaaatgggatggtaaaaccccattttaacagtacagagtgaattaaatgtgcatccattatcactatgcatatttacataatttgtcagtataaataaaacaattttattatttttatcttttaccatttttctatcttgttccatagtcttaattgctatttatatgagttttgaagaaaaccctatttttcagtggaagcattatggttctccattgttgactgaagattttacttcacctccagtatataaactacagtgtgctatgttacgaattctgaacttgcttatcttttgtttagaaaggcattatgtaaaacgaaataaagacgcacagactatggaaccagcgagcgtagaaaggagatcaccagtacatagaaaagagaatgagcaaagtataaaataggatgttaagtggaagtgattgcgctaatggtagaaaaatacattgaaagcattttaaaggtaaatactcactgtttttttcagcgagaaattaggcaacagctagcagaaaaagctaaagctggagaactaaaagtcgtcaatggagcagcagcgtcccagcctccatcaaaacgaaaacggcgttgggatcaaacagctgatcagactcctggtgccactcccaaaaaactatcaagttgggatcaggcagaggtaatttctttttgttttttgttattactgttaaaaaatttttttctgttatggactgtattagtctgcttgggttgccatagcaagataccacagactgggtgccttaaacaacagacattgatttttttcgcagttctggaggcttgaaatccaagatcagagtgccaacagggttggtgtttgttgagggctttcttgctgtgtcctcacatggcctttctctgtgccagcaagctctgttgtccatgctcttcctatgaggacatcagtcttactagattagggccccaccctcatgaccccatttgaccttaatttacctccctaaaggcccagtctccaaatacagtcacactgggggttaggacttcaacatacagattttgaggggcatgcaatttagtccatattatggaataaggaaagttctttttagaatattaaatacagacctgtaggtatgttacattgtggaggttgagtatgatatagtggtcttccttatcttctggatagataacttaagcccctcactactggcagcaaatagtcaatgctactcctttctcttggccagtctactctcctacctttttttttttttaaacatttgttttggaggctgaatgcattggctcatgcctataatctcagcactttgggggtccaggtgggaggattggttgagctcaaggagtttgagaccagcttgggcaacagagggagaccctgtctttacaaaaaaaaaaaaaaaaattagctgggcatagtggtgcatgcctgtagtcccagctactggggtatggggggtggtgggggctgaggtgggaggatcccttgatccccggaggtagaggttgcagtgagctatgatcatgccgctgcattccagcctggtgacagagtaagaccctgtctcaaaaaaacaaaacgatggcatctgtaaaatctttcttagaaatgtatttcctagttctgtagaaatggttgtattagatgttttctatcatttaataatatacttgcagactaaaagatataagtgctgcataaaagtagctaattatgttaaactgtcatatgtgcctttattatgtttgtcattatcctgaatagaaaggtctttaaaattgattttttagaaagcacttgaaaatatttaatcattgctagaaaaataaagcggctgggtgcggtggctcacgcttgtaatcccagcattttgggaggccaaggcaggcggatcttgaggtcaggagttcaagaccagcctggccaacgtagtgaaaccctgtctctactaaaaatacaaaaaaaattagctgggcatggtggcgtgtgcctgtagtaccagctacttgggaggctgagacaggagaatcacttgaacctgggaggtggaggttgtagtgagctgagattacgccaatgtgttccagcttgggcaacagagtgagactttgtctcaaaataaataaataaataattaaagctactcttttgcttatgcaccatttcccttcccctcttggagactctcatcatgtaccattaatttttttctccatactagattatcagcaccagcaaacatgttttatcgtgctttgtcttaataacagcaacaaaaaacctgcagaaaacatctcttaaccccattcacatccgcattcagctacaatcctgtttctatatctttttagaacaaagtgctttgttctttgtttttttcttgaggtagggtctcattctgttgccaaggttggagtgcaggggcgcaataatagctcactgcagcctccacctcctgggctcaaatgatcttcccacctcaaccccccaagtagatgggactatagacacgtaccaccatgcctggctagttttttatttttttgtagaaacgggtctcaccatgttgtccaggctgatctcaaactcctgggcttaagcagtccatctgcctcagccttgcaaagtgctgggattacaggcatgagccacagatgccttgaatgagttgtttgtacttgctttccctgcttacatagttctagtcttatgtaatttctagtctttttatgtagttctagtctattccacctaaaatctcttgtcaaggtcacttggcccctaattgataaatttaaaaagtgtattttcatttctcacatgatttagcataccaagaatatttgacatttcttcatacccttttctctctcagtctctgggttttagtctttcttgcttcccctcaaaatctcagtggctactactccttagtcttgtttgctggatcctcattcttcttacctctgaactttggggtgttatagtagtgctgagtcttttggattcttttccatctgtgctcaatccctaggatctcattcttgtctttatgtaccgtttgtataagatagctgccaaacttacaaatctgtcctctgcttctgttccgcagacttgcgtatttgttcagcttgacgtatttatttggctgttcaatgggcatttcaaaatcagtatttctaaaagaaaattattgattttcttcgcatacatgtcttctcagtcctgctcccctggtctttcctgtgtctgtttggctcaagcaaaacattaagggctgggcagagtggctcaggcctgtaatcccagcactttgggaggccaaggtgggtggatcatttgaggtcaggcgttcaagaccagcttggccaacatggtgaaaaccccgactctagttaaaatacaaaaattagcccggcatgttggcgggcacctgtaatcccagctacttgggaggctgaggcaggagaatcacttgaacccgggaggtggaggttgcagtgagaggagatcgtgccactgcacttcagtcttgcgacacagcgagactctgtctcaaaaaaaaaaacaaaacaaaacaagaacaacaataacaaaaaaacatttagggagtcatcctatactccttattctcttaccccatatcctacccatatacattttcttggcatcacctttaaatatattaaagagctagccacttttcatgatctctgcctctaccatcctagtctaaatcactattagctctagcctagactgctatagatttaatttataaaccaagaaaaaacctatcagatcatgcagtatttccccaaacccagaacagggatatataaaacctaatacataatttctaaacaatttagtttaaggtgaggacttctcatttgcttggttatatgtaggagggatttggttctgttcaatataagtaactactagctttatttttaattttaattttaatttttgagacagagtcttgctctgtcacccatgggtagctggagtgcattggtgcaatctcagctcattgcaatctccgcctcccagactcaagtgactcttgtgcctcagcctcccaagtagctgggattacaggcatgtgccatcacgcccagctaatttttgtatttttagtagagacagggtttctccatgttggccaggctggtcttgaacccctggcctcaagtgatccccccaccttggcctcccaaagtggtgggattacagttatgagccaccatgtccagccaagtaactagtaactttaattctatggatgacattcagtactaagtttgagaaagagaaagaagtttgtcctcctgttaattgtttaactgtatgcacataactagacacaagagctgatatctttttttttttttttttttttttttgagacaacagtcttgctctgtcacccagactagagtgatgattttggtcccctgcaacctccacctcccaggttcaagtgattctcctgcctcaccctcccaagtagctgggattacaggtgcctgccaccacgcctggctaacttttagtagagacagggtttcgcagtgttggccaggctggtgtcaaactcccaacctcaggtgacccacccgccttggcctcccaaggtgctgggattacacgtgtgagccactgcgcctggcatgagagctgatatgctgatatctttcttataaaaactacagagactcggccagacgcggtggctcacgcctgtaatcccagcactttgggaggccaagcgggcagatcacctgaggtcgagaggttgagaccatcctggccaacatggtgaaaccccgtctctactaaaaatacaaaaaattagccaggtgtggtggcaggcacctgtaatcccagctactcaggaggctgaggcaggagaatcgcttgaacccgggagacagaggttgcagggagccgagactgcgccattggactctggcctgggtgacagagtgagactctgtctcaaataaataaataaataaataaataaataaattaaatcagagactcagacttttttaaaaattctgttcatttaatacaactgtaccctagtcataattgtaggaattacaatagataatagaattcatttcccacctttccttataattcagcaggagggacagatgccagaatggagattttaaacaatttgtgtgctttatttacctaatccagaatttttcaaccctgagtaatagtaaaatctttttttttttttttttttgagacagagtctcactctcacccaggctggagtgcagtggcgtgatcttggcttgttgcaacctccgcctcctgggtttaggcaattctcatgcctcaccctcctgagtagctggaattacaggtgtgcaccaccacacccagctatttttatttatttttattttttatttttagtagagatgggcttttgccatgttggccaggctggtctcaactcctggcctcaattgatctgcctgcctggcctcccaaagtgctgggattacaggcatgaaccaccatgcccagccctgagtagtatgtttttaagggaaaaactattgagtcttaaacttgtttaatggtaccatgcaaatttgcaaacataaaggtagatgaagtccttgtgtctataagagctcccaactttcccaactttttttctttttttttttgagacggggtctcactctgtcgctcagcctggagtgcagtggtgctatctcggctcactgcaaactctgcctcccgggttcacaccattctcctgcctcagcctcccgagtagctgggactacaggcacccgctacaacgcctggctgattttttttgtattagtagagacggggtttcaccatgttagccaggatggtctctatctcctgaccttgtgatccgcccgcctcggcctctcaaagtgctgggattacgggtgtgagccaccgcacccggccaagagctcccaactttaaaacaaatatataaagtacattacacatcctgtggaatacttacgaggcagtatataagtacaaattttgttttctttacactcaactctatgctactttgatatgatactatctttattggtggagattcttttgctattacatgcagtcagttgtccaactattactctctgtttgaactactgtggaacccttgatgttacttaaacccacctctcagcctgggcagcatagtaagactcttatctctccaagaaataaaaaattaactgggtgtggtggtgtgcgcctgtatgtagtctcagctactcaggaggctgacatgggaggatcacttgagcttggtaggtggagactgcagtgagccgtgattgtgccactgcactcaacctgggcgacagagtgagaccctgtgtcaaaaaacaaatgaaaatccacctctcatcttttttcattaatcttgccagcatcagctagggtaatattatttggtacacatgtaactgtacatacaaactgatctcaagattgaaatcatgtgacggtatctggttataattacataggtggggcctggtgcggtggctcacgcctgtaatcccagcactttgggaggccaaggtgggtggatcacgaggtcaggagttcgagaccagcctgaccaacgtggagaaaccctgtttctactaaaaatacaaaaattagcccggcgtggtggcaggcacctgtaaccccagctactcaggaggctgaggctggagaatcacttgaacccgggaggcagaggttgctgtgagccaagatcatgccccggcactccagcctgggtgacagagcgagactctgtctcaaaaaaaaaaaaaaaaaaaaaattacataggtgatctaaagcatgtttatattaattaaacacacacaattaaacaaaacatcaacaggactattttcttagaacacacgaattccttctcccctaaatatgtgtctctaagccccactttgagaagtactaatgtaattagagctgaaagccttcgattactgatctagatagagaaacctcagtgatttcccccagtcacagagctattaatggcatatcaaagattcatatcgataatattttgtttttttaagctgggacttttgtcagtgcttcggattgaaactttacattgtttttaatcataaacatttgctagaaagtaggtatcttttcctataagtagaggtagtgtcttctaacttttcattaattatagtgcatagaactaaaacatttttcgtttaaagttttttattttatggaaattttctaattatttctgtgtgggtgtgtgaaataaaatttttttgcttttttaagacccctgggcatactccttccttaagatgggatgagacaccaggtcgtgcaaagggaagcgagactcctggagcaaccccaggctcaaaaatatgggatcctacacctagccacacaccagcgggagctgctactcctggacgaggtgatacaccaggccatgcgacaccaggccatggaggcgcaacttccagtgctcgtaaaaacagatgggatgaaacccccaaaacagagagaggtacttctagtggagtatgggtgtgtgggtgaaatgttgagtgatatgtttacagactgaaatgtgttctgtttattcctgggtggacacgtataaataaccagctgcttttgggggacagattaagaatagaacagttcatatgcctaattttttccacttcttgaagtggtttaagatttaaggaagaaccttaggtaaaagtaaagttagtaggctgtaacctttgattttgtgtgtttctttagagatagggtcttgctcactgtagccttgacatcctaggctcaagtgatcttcccacctcagccttcttagtagctgagactataggggcatgccaccatgcctggctaattttttttttttgagatggagttttgctcttgttgtccagactggaatgcaatggcgcgatctcagctcactgcaacctccacctcccggattcaggcgattctcctgcctcagccttcctgagtagctgggattacaggcatgcgccaccaagcccggctaattttgtatttttagtagagacggggtttctctatgctggtcaggcgggtctcgaactcccgacctcagatgatctgcccccctcggccttctgaaatgctgggattacaggcatgagccaccgcttccggcccctggctaatttttaaattttttgtagagacaatgtcttgcttcattgcccagcctggtgttgaactcctggtttcaagtgatctgccaaagtgttggaattacaggcatgaaccactgtgcctgacttaccctttgttttttattatggaaaaatgtacataacatcaaatttactttttttgttgttgttgttgagatggagtcttgctctgtcacccaagctagagtgcagtggtgcgctctcggctgactgcaacctctgcctccccagttcaagtgattcttttgcttcagcctcctgagtagctgggattacaagcgcccaccaccacacctggctactttttgtatttttagtagagacagagtttcatcatgttgatcaggctggtctcaaacaactttaaatgggtcaccattttaggaatattctattttagaacttgatagttttgagtataactgtttgagaataaagaatattattttaacagtgattaaaattgtggttttactcactctttctttttaaagatactcctgggcatggaagtggatgggctgagactcctcgaacagatcgaggtggagattctattggtgaaacaccgactcctggagccagtaaaagaaaatcacggtgggatgaaacaccagctagtcagatgggtggaagcactccagttctgacccctggaaagacaccaattggcacaccagccatgaacatggctacccctactccaggtagaactgtctcattggacataattgtttttttctccataaattatttaattttttcctttgtggtattctgtgtactattaatgtcaacttctagtaagaataaggagatatacaaatttaagtcttggtttgcgtttataatataaaagcaaataataggattgatcttaactgtcttttatctcaatgtatgtaataatacagtcttgtactatagattatataaaaatttgcttttcttggtaggtcacataatgagtatgactcctgaacagcttcaggcttggcggtgggaaagagaaattgatgagagaaatcgcccactttctgatgaggaattagatgctatgttcccagaaggatataaggtagtacatcaaatttgaatgaggtggtatttaggatatacatttattttaaatagtgaaaaaaattgattaagcttgcattggctttgcccaaatttactattgtcattccattctcttagctatttccaggtatatttcagttccatatagagatgaaaagaaatgaacaacatgaaacttttattactttttgaaagacatctcaaagacttgtgtatgggtctatcggttatactttgagaactgctggtgcttcagttttctagcagtcccaggaataagctcgttgagattgtggttattttacttggtgcttcaccaagattttatatacttaagtttatggattgtcagtggacacaaagaatttttttttttttttttttttaaaagatggagtctcactctgttgcccaggctgaagtgcagtggcgtgatttcggctcattgcaacctccgcctcccaggtttaagcgattctcttgcctgagtttcccaggtacctgggattacaggtgtccaccaccatgcccggctcatctttgtatttctagtagagacagggtttcgccatgctggccaggctgatcttgaactcctgacctcaagtgatccgtccctctcagcctcccaaagtgctgggattacaggcgtgagttaccgtgcctggcctttttatcatatcaacctaaaatttattttgaactataaaattttctaggtacctggtactcagggagtatttgttagtctgaagatagtctgtggtcgtatagctgacatcataaccagggggaaccagataactttgtaagtcatctgatggcctttacagagctttttctgttaatgactattgatttttaacagttcatgagtatgttttttgttgttttgttttgttttagagacagggtctcactgtgttgcccgtactggagagcagtggcgctgacatagctcactgcagccttggactcctgggctcaagtgatcctcctgcctcagcctcctgagtagctggggctacaggcatgcactaccatacctggctaatttttttattttttgtagatacgggagtctcactgtgttgccaggctggtctcaaactcctaggctcaaagtacccacattggcctcccacactgctggggattataggcatgagccactgtgtccagccttgttttgtttttgatcccctgttatttgtgttagagtatttgtttcaagtttgcatgaagttttgccttaaagctttttgtagcctccacataatgctgtgggtatagatagaactgtgggtatagatagacagaacttactgttgactgcctgcatgtctttctcagtcgtagtcattgatttccttagtgtcactaggaggaaaagtctcccttagtgaatcctactaataagcaacgtccttactagtgaatataaaaatatagtagacctttaaatccttaatggcaagtgatgcctgtggacttgacattgatttctctaggcctcacatctttagagtatccactttatccaaaatgcttagtggtctgtgctggtgcagacgttaactagcaacaaaaaatatctgtgcaaagtacagatagcatcaatataaatttaacataatttaatctttaatagacaaaacaattttaggctcactggagaagacgtgtataggtttattgacctctccaagaagttgtattttttttcttttactatatattaaattatataaaatagagatggggtcttgccatgttgccaaggctggtctcaaactcctgggctcaagcgttcctcccatctcagcctcccaaagtgctgtaatcctggcctgtccagaagttgttttttttttttgaagtctgttaccagctttaaaaaaaaaaaaaaaaatttctttgtgattttcagcctgcattttttgaaattttactcgttttgcaaaaggtttgatcaggtggtgtgtattttatagggctttggcaaacattttggttttagtttttatgcatttaactaatttttgtagagttttacacttaaaagattttgtagtgcaagaatttatgtgacattttaatactaaaatataattttttatgttttatgccccatatgagcttatttccattttatataaatcttatgaaacattatttgctcgactacagcattttttggaattgctgactcagattttataattttcaaatttttaataatttgacattttgacccacaacatgtttgaatgtgattagtattttctaagctcttctcttgtgtttcctcagttccctacaccacggatataatcagttagtggatgagaagtatatattttaagtttctaattaagatagaaaagcaagatagatgttgtctccaagcaaataaaaaccttattagctaataaactaaaatattttaagtgcaaatattgttcattatgctgttttttaagttaagaaaatcctaaactgtcttataacttttattaggtacttcctcctccagctggttatgttcctattcgaactccagctcgaaagctgacagctactccaacacctttgggtggtatgactggtttccacatgcaaactgaagatcgaactatgaaaagtgttaatgaccagccatctggaaatcttccatttttaaaacctgatgatattcaatactttgataaactattggtaagtgatactagcagaaataaactattaattgtgttccagaattataaatcataacatttcaacttcccttaacatttttttctttttactataggttgatgttgatgaatcaacacttagtccagaagagcaaaaagagagaaaaataatgaagttgcttttaaaaattaagaatggaacaccaccaatgagaaaggtaagtcccagtttgttaaattgtcatgtttgttgcttatttatttaattaaaaaaaatttgtagtttttgtagagacagggttttgccatgttgcccaggctggtctcaagtgatcctcctgctttggcctacctcccaaagtgctgggattacaggtgtgaggctctgcacctggccatgtttatatttttaaaaatgtttttaattttttcaccaggaaatgtagaccacaaaatagtagtgaactattggtatagcccgctgtggagtgtggtctttcctttactctcccaaatagcccaaattggtaaaggttatttagaaagccttccccataaaaaattaacttttggtagtgttatatttttgtgtttaggaatttataattactgtcttcatctttatcaaggacatagcatgaggggaaaaaatttatgtagtgtgaaggatttaacttttttgcttgtttttattattaatattatatatatatttttgagacagggtcttgctctcttgcccaggctggagtgcagtggtgtagtcttggctcactgcaacctccgcctcttgggttcaagcaattgtgcctcagccttccaagtagctgggactacaggcgtgcaccaccacgccctgctaatttctgtatttttagtagaaacggggtttcaccatgttggccaagctggtcttgaactcctggcctcaaatgattaacctgcctcggtctcccaaagtgctgggattacaggtgtgaaccaccatgcccagccgtttgtttttattattatttaaatactcctgggctcaagagatcctcctgcctcagtctccgaaagtgttgggattacaggtgtgagccaccgtgcccagctggagttagcacaaaattctttggttaaaaaagctaataaaaatgctgaaaaatgagattctcctttttgttcttaaggcctttaaaatatgcttaaagcttgaagataattagtgaaattatgtgagggattatgttaacactatatgtgccttggaaatggaagatacttcagggaaatgaaggcatttagtacatacctctgtggttcgttctattaaattactcttttgagttaacataaaatttctatcacaggaattgaaattccagagtaatttggatggaaatgaactcatgctgtctatgtaaaatgtgtgtaaaagtaaacactaaaatattaaagtatggaaaatcttctttgagtaatttgctcattcaaaatgtttgattataaagaaaccacacctattactctgctctttttcccaggctgcattgcgtcagattactgataaagctcgtgaatttggagctggtcctttgtttaatcagattcttcctctgctgatgtctcctacacttgaggatcaagagcgtcatttacttgtgaaagttattgataggatactgtacaaacttgatgacttagttcgtccatatgtgcataaggtttgttctccttaatagtgtttctgatagtttaaagttttaactttttcagttaatcaatgttaaatttacatattctcctagaccttttcctttgcacaagtacacaccatacatgtacaaatacagttttatacagatgtacagatgttagtttttgtcatctttgccgtaagaggttcatattatttaattttttcatttgcttttattaaaattagattaggatgagaacatatgagaactaacgccagttagtattaagaaccagttttttcaccgatggtaattatggtgcagtatgttcatagggtgtataagttatttctttaaaaaatattgaatattagtgtgtcactaatagctctcttaaattttcatctctgtctggagtttgaacttgtataattaaaattcttaaacagttcgtcccttgattaacaaaagtcctgataattctaaatttcttatgtatgtttaattctgtacatgagcatttcatcagtaattgatgtgaaagtgtagcttcttctcttttctctttttcagatcctcgtggtcattgaaccgctattgattgatgaagattactatgctagagtggaaggccgagagatcatttctaatttggcaaaggtatttacatttaatttctagagaagaaaatttatatctgtttatggaattgattatggaaagaaatggttgaagattaatattaccaactcatgactgtcctttctttgtttacattttaggctgctggtctggctactatgatctctaccatgagacctgatatagataacatggatgagtatgtccgtaacacaacagctagagcttttgctgttgtagcctctgccctgggcattccttctttattgcccttcttaaaagctgtgtgcaaaagcaagaagtcctggcaagcgagacacactggtattaagattgtacaacagatagctattcttatgggctgtgccatcttgccacatcttagaagtttagttgaaatcattgaacatggtaagttgtaatgtaactttgtctttttttttttttctttaggagacagggtcttactatgttgcccagactggactcaaacctttgggctcaagtgatcctcctgctcagcctccttagtagttgggactagaggtacacacacagcctgtccatgtttaataggacagctgtcctaaaatttgggctactgatttggggagataaatggaaaggcatagctctacaaactatagattttatgatgggtttgttatattatctgctgacaggctatggttcatgttttgcttttacctaattttgtttaatgtgaacatattctgcagtttggctgaatagttgatatattgagagaatctggatgatattgtgtaacttaggtaatgttggggcatagttaaaacctgtgtttggttttgtaggtcttgtggatgagcagcagaaagttcggaccatcagtgctttggccattgctgccttggctgaagcagcaactccttatggtatcgaatcttttgattctgtgttaaagcctttatggaagggtatccgccaacacagaggaaaggtaaatccaccaattaccttttgatttatcttcattaaagttaaggcgacataaatctaaattactaaagtacatatattttttatttaaaaatagggtttggctgctttcttgaaggctattgggtatcttattcctcttatggatgcagaatatgccaactactatactagagaagtgatgttaatccttattcgagaattccagtctcctgatgaggaaatgaaaaaaattgtgctgaaggtaattattccagatttgttaatgtaaactggatatgtttcatggttctaacagaattttaaagtgtttttaaaaatcaacaaaaaaaactgtatgttacacgaatactgtttgatttgtttttaaattttattttttaaaatcacgtaatcagcaatgagtattctcttcatttcaggtcagttgatttattattactatttcttaaccttttaggtggtaaaacagtgttgtgggacagatggtgtagaagcaaactacattaaaacagagattcttcctcccttttttaaacacttctggcagcacaggatggctttggatagaagaaattaccgacaggtaagcttttattgtaaaagaatattctcaaaagtcctttcaactcattctttagtcaacttgaatgcatgttgtatattattttttgccttgattatctcacagtttatatgtgcttgattatgaaaggagatggattttagtcagccagttaacttccattttgattttggattcgaggattaatacttaactgtgaattgatgtttttgtctggtacattcagtgctttatctggctagatgaaacatggacacagttttaccttgaatcatgttaacttatactcatttttcgatgtttggtcacttttctcttcaactttaataagtagcaatgaatcattcttaccatgtttattagctaaataagtagtatatttagagtatttggttttcatgatgttgctttatttccttggaaaagcagtctaaaaggttttttgtttttctgttctttttagttagttgatactactgtggagttggcaaacaaagtaggtgcagcagaaattatatccaggattgtggatgatctgaaagatgaagccgaacagtacagaaaaatggtgatggagacaattgagaaaattatgggtaatttgggagcagcagatattgatcataaacttgaagaacaactgattgatggtattctttatgctttccaagaacagactacagaggtaatgaaaactattatggcacattgctactaactttaatatttgtagaaaagcatattagttgtcaagcaaatttttctagaatgcaattgaatttgaagtaatttttaaaatgctcagggggttaccaaacgatattattctacctcaagttttaagatttgatgtaaaaaaacagcaacagatgtttgggtggttagaaaaggttgactcttagtgtatttcaactgtgcagtcataaaccaaatgaatatgtcttttttttaaatttattttttaggactcagtaatgttgaacggctttggcacagtggttaatgctcttggcaaacgagtcaaaccatacttgcctcagatctgtggtacagttttgtggcgtttaaataacaaatctgctaaagttaggcaacaggcagctgacttgatttctcgaactgctgttgtcatgaagacttgtcaagaggtaaacttttgcatcaaattcttacttcttccccaaatagtttaatgtaaatagtaacattttaacattatttgttttattataggaaaaattgatgggacacttgggtgttgtattgtatgagtatttgggtgaagagtaccctgaagtattgggcagcattcttggagcactgaaggccattgtaaatgtcataggtatggaatttgctggttacataagactttttcttgttttaactttctttttcttttttttttcgtaagtaaaatcttatttaggagttgggaggtttttgttagctttttaaaagcacttcaagattgtagttaagtcgggggatgtcacaaaagtcttgtttcccaaaacccatttatattttactttaaagagaaaaaaagggtaagtttaggacctacatgagaggtgggagtagtcacagctcaaaaagctttgaagtcatggaatacaggaccctagtagaaatagtgtgtcaggtgaaatatattctgccatgttctgctgcggggtgaaaaggcaatcctaaagttttaatttagttttagaacaatatatatatatttaaaccttgaattagcgattatgacaggttttttgcttgtgatcagtttcaaggaatataatcagagaaaagtgaactgatcaaattattgtctttatttacatacccaagttttaaatactgtccattcagttttctaacccgtaatacagctatattacaaatggtcttggatatggagagcaaagtttttattccattagggcagtatgaagaaaacaaatggtttcttatatttaatcttttttttaaattgattataactgttgccaaatgtaactcctgcagagtttagaggctctgataacctagcagaagtcaaaatagctgactgtgagagagccactgtttgtttttcctcattttgtaagaggtatttatgcgatacacatgtttaaactattgtatttgcatataatctccaatgcaaccctatcctctatacttgaagatagctacttgccagaaatcatttacatggccacttgtagggctctggtttgcagggagttttaaaccatattgtttgtcagagaggcatgggaagagcaatggagagagaatcagagcaagtgattctagttagagcaagagcccctgccagctgcctccttttttctctcttcgtaggtgtaaataccatccaagtatgccctgcattgcagggagactggagatctgtttgttttgcagttaaagggtaagggggccctttctggaaatttttgctttatttacatgtttacctctggcttatattgttgtgtatggccggaacataagctgtccccatatttaaatgtcagtgcacaaatatatttcagaatcaaacaaaacttttttgttattgagtatccatcatcctttaaatcacttcactgatttctttcatttctatacataatcaaaaattgttaataaaagttaaaaacgattctgagtctggtcagaatctggggctttctctttccctaggcagctgtaagtttatatttatacatagtcatatatgacattgctaagtaaaaggaaagtgaacaaaagttgcaattcaaatgttgatagtttattgacttctattaaataggtatgcataagatgactccaccaattaaagatctgctgcctagactcacccccatcttaaagaacagacatgaaaaagtacaagagaattgtattgatcttgttggtcgtattgctgacaggtaagcagattacccacacatttttataagcagtatcaattttcaaatttggccactttgtattcaattagcaaaatttccttaatgtcactaatataagtgtaaaagattaggctatccagtattaatattttcagtcaaatttcaatttggatggtaatgggggtggcaatgtaattttaggactaaacctgtaagactaactcttcagcctttctgaagagtagtaagttttatttaatgcttaaaatgagttaatcatgtttttagaactgaatttgcaaatttatccttaaaacttttcttaatgtcacaataattaatatgtgtattttttaaattaggggagctgaatatgtatctgcaagagagtggatgaggatttgctttgagcttttagagctcttaaaagcccacaaaaaggctattcgtagagccacagtcaacacatttggttatattgcaaaggccattgggtgagtgttatttacttccattaaaaaaaaattactttataataaatgcattgtggtattggaaaatacttccatgtagaaatctcttttgaggcatggtctgaataatgcagttgagaaagctcttcatttaaccactgaccttctgattattggaatgtattgttaaattctttttttttttttttgagacggagtctcgctctgtcaccctggctggaatgcagtgattcaatctcggctcactgcaaccaccgcctcccaggtgcaggcgattctcctgcctcagcctccccagtagctgggactacaggcatgcaccatcacgcctaggtaatttttgtattttttagtagagatgaggtttcaccatgtgggccaggctgttctcgaactcctggcctcaagtgagccacctatcttggcctccaaaagtgctgagattacaggcgtgagccactgcgcacagcctgtagtcttaaattcttaattaaaatagatggaagggtttaagacttactttaattacactttaggtgttttcagggagggaaacagtaccattttcagaaaaatttaaaatgtgtgctggtcttctaggatattgcttaaactattaaggcaagtttttagtctcacaacatcatttaagtctcatattaatgctttatatgttttaaaaatcttattatggattgtttgagcccaggagttcaaggttacagtgagctgtgattgtgccactgcactccaacctgggcatctgggcacctgggcgacagggcgagaccctgtctctaaaaaaaacaacaaaaatgctattctaacattactccacggatttaactgaattatagctgctttgtgagataatctgtggtggtttgcttccactccatcacaactaaaataggaaaaaacagtcattgtagaaaacgagacaccacatgtccatgtggcttaagaaaatgttttcaaggaatatgacatgtgaatgtctgattaattaggtagaattataccagacaacaccccctcccctaacacatactcattcctctcctattttggtagcataatatttatttcagtggtgatggagtcaagacatttgcaagaatagcttttgtgcaagcttttagccccaatgtccctctaagtttatgtcaaagaagaggcaaatagatgcagcaaattgtgtggaggaaagataaaaggaaaagagattttatatggtttataatgggagactacaattcggatattcccctcaccactcctacctagttctagtgcccattttgacaactcagagaattactcactttaaacatttagttaaatttctttatatttcagaaagtttaagaactgaaaattacatttgaaaattgagttaacttaaaatgtttagaatttgtacagctatgacctctgatcttctcagagtcaacttttttaaactgaccttttaatttatatgaaatttttatatagaaacaacaatgccagtgacttgaaagtattgcattttataaaattactgagttccctgtaattattaatacttaaaagttttcatctttatgcatttttttcttccataaatttttgacttataatgtaacagcttgttgacccatttgtttttttcagccctcatgatgtattggctacacttctgaacaacctcaaagttcaagaaaggcagaacagagtttgtaccactgtagcaatagctattgttgcagaaacatgttcaccctttacagtactccctgccttaatgaatgaatacagagttcctgaactgaatgttcaaaatggagtgttaaaatcgctttccttcttgtttgaatatattggtgaaatgggaaaagactacatttatgccgtaacaccgttacttgaagatgctttaatggataggtaagtgacttgaactaaatacaagaaaataattataactcttccttcattttagaacatcgtgaaatagttattagatgactttttactgtaacatagttaaagaaaaaattgctagcctggagtcttaggacattgtaaatatgacttattattttaaaataatgtgcacgctgtagtgattccccagactttctttcaagttctcccatcccctttaaatctcttttcttatctattgaattaaataaattttgaaaagggttgattgataatcttttgttttcaaaattggtcaaatatagttagaggacatgatgagcagaagctaatgggcagagacctgatctttttagctttttgttgcaggtaaaggacaggtttctcttaagcctttgaattactgaacaatagcctgccagaactacactcttcctgttgcaggtccctggtgtttcagatggtgaccaccagtagagtactgttcaagttatttgacagattgctaataccttttaccatctagactgacgtacagccttaaacacagaagtaactgaagaaagtgcaaaggaaagtgcaagcgtagtaagggaaagaactgtttcactggctctttatttttcaggagagataagttactcaaatcagagcccattcaaatacttttgtgtacagatttaagttattcattaataaccttctgaaagggagacttaactggactcgacaagttaccagaacgtatatatgcaagacatggagacaaagtaagaattgtgctctgtaaaagcctggctgcccagggctaaaaaaaatgagggtaaatcacccttgctctgtattccttagtgagaagtgcctgagcaccatagtgcagtcattaatgtatgtcacctgcagttaaaaacttttttctgtgtactaagaagttaagggagtaattccattgaaaattactaaatgaaaagaatacttcagttggaaattttattaatgaggaatactgcttcattcttatgctgaatgacattattaaaataaataggaaaaacaagaatattagtaaatacaagtatttttctcttttctctttgatatggtctgtgttactatctttggagcaaagtgtggaaactcatttttttttttggacggagttttgctgtgtcacccagtctggagtgcagtggcatgatctcggcatactgcaacctctgcctcccaggttcaagtgattcttctgcctcagcctcccaagtagctgggactacaggcgtgtgccaccatggccggctaattttttgtatttttggcaaagatagggtttcaccgtgttagccaggatggtctccatctcctgacctcgtgatctacccgccttggcatcccaaagtgctgggattacaggcgtgagccaccgtgcccggcctggaaactcattttcatacccattgcaaatcccattgtgttagccagcgagctcatacttggttgaataattgtggaatgaataactcattgatttttcatgaacataaattcagttaacataggagatgatagttatttctgtaggtttgaaaaaattattgatcttttgttcctctccccaaattgagttattgaatgaaacttaaactttacgtgagaagggaatttaactttaatttagttacctattatataccagtatctgaagtactagtaaagatttaagtttataatcaagagtttaaaatgatttgggaggccaaggtgggaggatcgcttgaccccaggagctcaagaccagcttgggcaacatagcgaggccttccctatctctaacaataacaataaaaaagaaaaataatttttcttttttttttgagacagagtttcactcttgttgcctaggctggagtgcagtggcacaatctcgactcactgcaacctccaccccccaggttcaagtgattctcctgcctcagcctcctgagtagctgggattacaggcgtgtgccaaaatgccctactaatttttgtatgtttagtagagacggggtttcaccacgctggccaggctggtcttgacctcctgacctcaggtgatccatctgcctcggcctcccaaagcgctaggattacagacatgagccaccatgtccggcctgaaaaataattttaaaaaagagaaaaaagtttaaaatattactgtttattattcatatgttagaaccacatgcctcctctttagtagcataataaagccaagacttcacagagccctcctgctcatattccttttaaaagttaattagatggccaggcacggtggctcacgcctgtaatccctgtactttgggaggctgaggcgggcagatcacaaggtcaagagatcgagaccatcctggccaacatggtgaaaccctgtctctactaaaaatacaaaaattaggcaggcgtggtggcacacacctgtaattccagctactcaggaggctgaggcaggcgaattgcttgaacctgggaggcagaggttgcagtgagccaagatcacaccactgcactccagtctgggtgacagagtaagactccgtctcaaaaaaaaaaaaaaagttaattagaaaatatggcttaattgttgagccatagtgaatcttgagtaattaaagcaaaagtcctcaagtggaagctattctaaaggatatattagatatatgaaatgttagatgacttcacatctattcagtgcactgaattagtaatttagtttctaatgggccggtgaatatttcctgtaaaactaagactgtatctttaaggatttttccccctttcttataagaccgcatcttaaaggacttttttcccttccgttgtagagaccttgtacacagacagacggctagtgcagtggtacagcacatgtcacttggggtttatggatttggttgtgaagattcgctgaatcacttgttgaactatgtatggcccaatgtatttgagacatctcctcatgtaattcaggcagttatgggagccctagagggcctgagagttgctattggaccatgtagaatgttgcaatattgtttacaggtaagttaaagattttttttttaatcggtgattttttttttttaaataatactttaagttctgggatacttgtgctgaacgtgcaggtttgttacataggtatatatgtgccatggtggtttgctgcacctatcaacctgtcatctaggttttaagccctgcatgcattaggtatttgtcctaatgctctccctcccctttccccccactgcctgtaagttaaagatttttaaaagattacttagcaagaaaagtagtgtattgaattaagttacttttcctgattatcattagggtatattgtgccatatggtagaacaattgtgtggtgacaggggagtgagttttgtctcttccattgcagcttaagagcagtgtaattagtaggttcccccccccccaactccccaagggaaataattttgaatcatatctttatgttttcttaattttgttaaggttatgtatgttcttaaaaattgaaataatgtaaccgttcaattattttagggtctgtttcacccagcccggaaagtcagagatgtatattggaaaatttacaactccatctacattggttcccaggacgctctcatagcacattacccaagaatctacaacgatgataagaacacctatattcgttatgaacttgactatatcttataattttattgtttattttgtgtttaatgcacagctacttcacaccttaaacttgctttgatttggtgatgtaaacttttaaacattgcagatcagtgtagaactggtcatagaggaagagctagaaatccagtagcatgatttttaaataacctgtctttgtttttgatgttaaacagtaaatgccagtagtgaccaagaacacagtgattatatacactatactggagggatttcatttttaattcatctttatgaagatttagaactcattccttgtgtttaaagggaatgtttaattgagaaataaacatttgtgtacaaaatgctaa

SEQ ID NO: 2 - DNA, 4338 bp, Homo sapiens
agagtgcagcccccagctatttttctccgtggcggcggcgacgagcggaagttcttgggagcgccagttccgtctgtgtgttcgagtggacaaaatggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggagggaaaacccctgatcctaaaatgaatgctaggacttacatggatgtaatgcgagaacaacacttgactaaagaagaacgagaaattaggcaacagctagcagaaaaagctaaagctggagaactaaaagtcgtcaatggagcagcagcgtcccagcctccatcaaaacgaaaacggcgttgggatcaaacagctgatcagactcctggtgccactcccaaaaaactatcaagttgggatcaggcagagacccctgggcatactccttccttaagatgggatgagacaccaggtcgtgcaaagggaagcgagactcctggagcaaccccaggctcaaaaatatgggatcctacacctagccacacaccagcgggagctgctactcctggacgaggtgatacaccaggccatgcgacaccaggccatggaggcgcaacttccagtgctcgtaaaaacagatgggatgaaacccccaaaacagagagagatactcctgggcatggaagtggatgggctgagactcctcgaacagatcgaggtggagattctattggtgaaacaccgactcctggagccagtaaaagaaaatcacggtgggatgaaacaccagctagtcagatgggtggaagcactccagttctgacccctggaaagacaccaattggcacaccagccatgaacatggctacccctactccaggtcacataatgagtatgactcctgaacagcttcaggcttggcggtgggaaagagaaattgatgagagaaatcgcccactttctgatgaggaattagatgctatgttcccagaaggatataaggtacttcctcctccagctggttatgttcctattcgaactccagctcgaaagctgacagctactccaacacctttgggtggtatgactggtttccacatgcaaactgaagatcgaactatgaaaagtgttaatgaccagccatctggaaatcttccatttttaaaacctgatgatattcaatactttgataaactattggttgatgttgatgaatcaacacttagtccagaagagcaaaaagagagaaaaataatgaagttgcttttaaaaattaagaatggaacaccaccaatgagaaaggctgcattgcgtcagattactgataaagctcgtgaatttggagctggtcctttgtttaatcagattcttcctctgctgatgtctcctacacttgaggatcaagagcgtcatttacttgtgaaagttattgataggatactgtacaaacttgatgacttagttcgtccatatgtgcataagatcctcgtggtcattgaaccgctattgattgatgaagattactatgctagagtggaaggccgagagatcatttctaatttggcaaaggctgctggtctggctactatgatctctaccatgagacctgatatagataacatggatgagtatgtccgtaacacaacagctagagcttttgctgttgtagcctctgccctgggcattccttctttattgcccttcttaaaagctgtgtgcaaaagcaagaagtcctggcaagcgagacacactggtattaagattgtacaacagatagctattcttatgggctgtgccatcttgccacatcttagaagtttagttgaaatcattgaacatggtcttgtggatgagcagcagaaagttcggaccatcagtgctttggccattgctgccttggctgaagcagcaactccttatggtatcgaatcttttgattctgtgttaaagcctttatggaagggtatccgccaacacagaggaaagggtttggctgctttcttgaaggctattgggtatcttattcctcttatggatgcagaatatgccaactactatactagagaagtgatgttaatccttattcgagaattccagtctcctgatgaggaaatgaaaaaaattgtgctgaaggtggtaaaacagtgttgtgggacagatggtgtagaagcaaactacattaaaacagagattcttcctcccttttttaaacacttctggcagcacaggatggctttggatagaagaaattaccgacagttagttgatactactgtggagttggcaaacaaagtaggtgcagcagaaattatatccaggattgtggatgatctgaaagatgaagccgaacagtacagaaaaatggtgatggagacaattgagaaaattatgggtaatttgggagcagcagatattgatcataaacttgaagaacaactgattgatggtattctttatgctttccaagaacagactacagaggactcagtaatgttgaacggctttggcacagtggttaatgctcttggcaaacgagtcaaaccatacttgcctcagatctgtggtacagttttgtggcgtttaaataacaaatctgctaaagttaggcaacaggcagctgacttgatttctcgaactgctgttgtcatgaagacttgtcaagaggaaaaattgatgggacacttgggtgttgtattgtatgagtatttgggtgaagagtaccctgaagtattgggcagcattcttggagcactgaaggccattgtaaatgtcataggtatgcataagatgactccaccaattaaagatctgctgcctagactcacccccatcttaaagaacagacatgaaaaagtacaagagaattgtattgatcttgttggtcgtattgctgacaggggagctgaatatgtatctgcaagagagtggatgaggatttgctttgagcttttagagctcttaaaagcccacaaaaaggctattcgtagagccacagtcaacacatttggttatattgcaaaggccattggccctcatgatgtattggctacacttctgaacaacctcaaagttcaagaaaggcagaacagagtttgtaccactgtagcaatagctattgttgcagaaacatgttcaccctttacagtactccctgccttaatgaatgaatacagagttcctgaactgaatgttcaaaatggagtgttaaaatcgctttccttcttgtttgaatatattggtgaaatgggaaaagactacatttatgccgtaacaccgttacttgaagatgctttaatggatagagaccttgtacacagacagacggctagtgcagtggtacagcacatgtcacttggggtttatggatttggttgtgaagattcgctgaatcacttgttgaactatgtatggcccaatgtatttgagacatctcctcatgtaattcaggcagttatgggagccctagagggcctgagagttgctattggaccatgtagaatgttgcaatattgtttacagggtctgtttcacccagcccggaaagtcagagatgtatattggaaaatttacaactccatctacattggttcccaggacgctctcatagcacattacccaagaatctacaacgatgataagaacacctatattcgttatgaacttgactatatcttataattttattgtttattttgtgtttaatgcacagctacttcacaccttaaacttgctttgatttggtgatgtaaacttttaaacattgcagatcagtgtagaactggtcatagaggaagagctagaaatccagtagcatgatttttaaataacctgtctttgtttttgatgttaaacagtaaatgccagtagtgaccaagaacacagtgattatatacactatactggagggatttcatttttaattcatctttatgaagatttagaactcattccttgtgtttaaagggaatgtttaattgagaaataaacatttgtgtacaaaatgctaa

SEQ ID NO: 3 - DNA, 665 bp, Homo sapiens
agagtgcagcccccagctatttttctccgtggcggcggcgacgagcggaagttcttgggagcgccagttccgtctgtgtgttcgagtggacaaaatggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggcttctattctgctgcttgaagtcagaactgctgatggagacaaaggcacgaaagtgtacgtattccggattagcaacccaggaacccatcacttctgaagactctaaactgtgctgtcattttgtttttatatgcattaaaatatttgttttaaa

SEQ ID NO: 4 - DNA, 2141 bp, Homo sapiens
agagtgcagcccccagctatttttctccgtggcggcggcgacgagcggaagttcttgggagcgccagttccgtctgtgtgttcgagtggacaaaatggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggtaattctttcccacttttctataagtattcagaaatttatttgtattaagttgtctttagccctttgattttttttggcatgcttctgaatttgcttttctttttaaaaatcccccttactatagatttgtatagatgtggttgaagtcacagatgccatattcttcatctttataatgcaagttttctttccaggtagaaggacaagaaaaaaatgagccaatgccatagaaacgcaaagggttaaaggttttacttttacttttgttttgggtacgctttctatacttggatcaaatgtatttttctgctcaaaatactttcaaattattgaaagttttcattccaaatcctagatttttaggattgttacccttgagaaactgtttctcacactagagctccccccatagtgctctaaatatagcttagtaaaggcacctactttgctctaagcatacttaaagttaaaacaaaagtatgtatacgaagagtttgatatttttaatagttggagtctgttttgctaattttagtgtgcaagtcagcacaaggggcatagtgagcctgatggaagctttcctcaggtgagtgagtttcacaatcaaaattaggtgcctgtttcttaaaattagattctgctgcaggtttacatactagacttttcagcctgcatgaattagtagggatattctttttttacttgcataattgtaattgatttaaacatgtatgccagaattcacaggcccatactaactgtccttaatttctttcctacagcagtactgctatatgccagtcctgtctgcattcttaagggtgcagttcaacacatcctctctagattatggtgaaaaagtattccaaaggaagtcttatcagagctagtgtcagaaagaatgacactaccaattgggcatgatcttcagcataggaaatgtcttagaattttattattttttcctaaattgtgatgctatactacttgtatagctagttaccacatttctaaagcatagtgtgcctctgtgaatcttgttatatactgtttgggcttctgtaagatattttgaagtttgaaaagcaaatactgaaactttaaccaagagactgacacatctctttgtacaccacgttgcttctgtgtgcatcataataactggtagaagtaatatatttaaagtaatatgtttccagttttaatttatgctttctaagaaataaaaatatgttttctgggtcaacaaaacttaaagcatgaagccctttttagtaacgtgacatcagaataagattataggtatatttttaaaatcagattttctaaactacaattgttttagaacactgtatgaagaacttatctagtcatagttatttgtagtcaggattttaacagcttgcaacaggtaatgttttgaatataaatatctttccaaagtgttactaatggtaaagagataattttctaggcttctattctgctgcttgaagtcagaactgctgatggagacaaaggcacgaaagtgtacgtattccggattagcaacccaggaacccatcacttctgaagactctaaactgtgctgtcattttgtttttatatgcattaaaatatttgttttaaa

SEQ ID NO: 5 - protein, 1304 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNARTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGREIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL

SEQ ID NO: 6 - protein, 144 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGFYSAA

SEQ ID NO: 7 - protein, 154 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGNSFPLFYKYSEIYLY

SEQ ID NO: 8 - DNA, 23 bp, Homo sapiens
caactccttatggtatcgaatct

SEQ ID NO: 9 - DNA, 21 bp, Homo sapiens
cctcgattctacaggttatta

SEQ ID NO: 10 - DNA, 58 bp, Artificial Sequence
  lacZ shRNA sense strand
ccggtgttcgcattatccgaaccatctcgagatggttcggataatgcgaacatttttg

SEQ ID NO: 11 - DNA, 58 bp, Artificial Sequence
  lacZ shRNA antisense strand
aattcaaaaatgttcgcattatccgaaccatctcgagatggttcggataatgcgaaca

SEQ ID NO: 12 - DNA, 58 bp, Artificial Sequence
  TR-shSF3B1#3 sense strand
ccggcaactccttatggtatcgaatctcgagattcgataccataaggagttgtttttg

SEQ ID NO: 13 - DNA, 58 bp, Artificial Sequence
  TR-shSF3B1#3 antisense strand
aattcaaaaacaactccttatggtatcgaatctcgagattcgataccataaggagttg

SEQ ID NO: 14 - DNA, 58 bp, Artificial Sequence
  TR-shSF3B1#5 sense strand
ccggcctcgattctacaggttattactcgagtaataacctgtagaatcgaggtttttg

SEQ ID NO: 15 - DNA, 58 bp, Artificial Sequence
  TR-shSF3B1#5 antisense strand
aattcaaaaacctcgattctacaggttattactcgagtaataacctgtagaatcgagg

SEQ ID NO: 16 - DNA, 21 bp, Homo sapiens
cgctattgattgatgaagatt

SEQ ID NO: 17 - DNA, 21 bp, Homo sapiens
caactccttatggtatcgaat

SEQ ID NO: 18 - DNA, 21 bp, Homo sapiens
tgctttgatttggtgatgtaa

SEQ ID NO: 19 - DNA, 21 bp, Homo sapiens
cctcgattctacaggttatta

SEQ ID NO: 20 - DNA, 58 bp, Artificial Sequence
  RNAi consortium shSF3B1 #2 (TRCN0000320576)      hairpin sequence
ccggcgctattgattgatgaagattctcgagaatcttcatcaatcaatagcgtttttg

SEQ ID NO: 21 - DNA, 58 bp, Artificial Sequence; identical to SEQ ID NO: 12
  RNAi consortium shSF3B1 #3 (TRCN0000320566)      hairpin sequence

SEQ ID NO: 22 - DNA, 58 bp, Artificial Sequence
  RNAi consortium shSF3B1 #4 (TRCN0000350273)      hairpin sequence
ccggtgctttgatttggtgatgtaactcgagttacatcaccaaatcaaagcatttttg

SEQ ID NO: 23 - DNA, 58 bp, Artificial Sequence; identical to SEQ ID NO: 14
  RNAi consortium shSF3B1 #5 (TRCN0000320636)      hairpin sequence

SEQ ID NO: 24 - DNA, 20 bp, Homo sapiens
ccaaagattgcagaccggga

SEQ ID NO: 25 - DNA, 20 bp, Homo sapiens
tcaggggttttccctccatc

(19) United States
(12) Patent Application Publication
(10) Pub. No.: US 2019/0147975 A1
(43) Pub. Date: May 16, 2019

(54) SF3B1 SUPPRESSION AS A THERAPY FOR TUMORS HARBORING SF3B1 COPY LOSS

(71) Applicant: DANA-FARBER CANCER INSTITUTE, INC., BOSTON, MA (US)

(72) Inventors: RAMEEN BEROUKHIM, BROOKLINE, MA (US); BRENTON PAOLELLA, WEST ROXBURY, MA (US); WILLIAM J. GIBSON, BOSTON, MA (US)

(21) Appl. No.: 16/091,731

(22) PCT Filed: Apr. 7, 2017
(86) PCT No.: PCT/US2017/026693

§ 371 Date: Oct. 5, 2018

Related U.S. Application Data
(60) Provisional application No. 62/319,490, filed on Apr. 7, 2016.

(51) Int. Cl. G16B 20/20; G16H 50/20; C12Q 1/6886; G16H 50/30; G06F 17/18; G16B 20/10

(52) U.S. Cl. CPC G16B 20/20; G16H 50/20; C12Q 1/6886; C12Q 2600/156; G06F 17/18; G16B 20/10; C12Q 2600/106; G16H 50/30

(57) ABSTRACT

The present invention provides an association between copy loss of SF3B1 in cancer and sensitivity to SF3B1 suppression. Cancer cells harboring partial SF3B1 copy-loss are more sensitive because they lack a reservoir of SF3b complex that protects cells with normal SF3B1 copy number from cell death upon SF3B1 suppression. The invention also provides methods for treating cancer, especially cancer with SF3B1 copy loss, by suppressing the expression or activity of SF3B1.

### RELATED APPLICATIONS

This application claims the benefit of U.S. Provisional Application No. 62/319,490, filed Apr. 7, 2016, which is incorporated by reference herein in its entirety.

### GOVERNMENT RIGHTS

This invention was made with government support under grant numbers R01 CA188228, F32 CA180653 and F30 CA192725 awarded by the National Institutes of Health. The Government has certain rights in the invention.

### BACKGROUND

Genomic instability is a hallmark of cancer resulting in widespread somatic copy number alterations (SCNAs), which affect large fractions of the genome. SCNA-related dependencies can be categorized into four classes. First, cells may be dependent upon amplified genes, as has been noted with several amplified oncogenes. Second, cells may be dependent on genes that have undergone partial copy loss. This “CYCLOPS” (Copy-number alterations Yielding Cancer Liabilities Owing to Partial losS) phenotype has been validated for three genes: PSMC2, POLR2A, and CSNK1A1. Third, copy gain may be associated with dependencies on genes outside the amplicon. Fourth, copy loss may be associated with dependencies on genes outside the deletion, as has been described for pairs of paralogs such as ENO1 and ENO2. The relative frequency of each of the four classes and their general features is largely unknown.

The present invention used a genome-scale shRNA viability screen to perform an unbiased analysis of copy-number associated gene-dependency interactions. Among all copy-number associated dependencies, the most highly enriched subclass were “CYCLOPS” genes, whose hemizygous loss sensitizes cells to their further suppression. The invention identified a splicing factor SF3B1 as a CYCLOPS gene and revealed the underlying mechanism.

### SUMMARY

The present disclosure provides a method for determining the likelihood that a subject with cancer responds to an SF3B1 suppression treatment, comprising measuring the copy number of SF3B1 in a sample comprising cells from the subject, wherein the likelihood is increased if the copy number of SF3B1 in the sample from the subject is smaller than the ploidy of the cells in the sample.

In some embodiments, the cancer is selected from the group consisting of breast cancer, hematopoietic cancer, bladder cancer and kidney cancer. In some embodiments, the cancer is selected from the group consisting of acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, chronic myelogenous leukemia, colon adenocarcinoma, esophageal carcinoma, glioblastoma multiforme, head and neck squamous cell carcinoma, chromophobe renal cell carcinoma, renal clear cell carcinoma, renal papillary cell carcinoma, hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma.

In some embodiments, the sample comprises a cancerous lesion. In some embodiments, the sample comprises circulating tumor cells.

In some embodiments, measuring the copy number of SF3B1 comprises comparative genomic hybridization (CGH). In some embodiments, measuring the copy number of SF3B1 comprises fluorescence in situ hybridization (FISH). In some embodiments, measuring the copy number of SF3B1 comprises amplifying a genomic sequence comprising at least 20 nucleotides of SF3B1.

In some embodiments, measuring the copy number of SF3B1 comprises DNA sequencing. In one embodiment, DNA sequencing comprises whole-genome sequencing. In another embodiment, DNA sequencing comprises whole-exome sequencing.

In some embodiments, the copy number of SF3B1 in the sample is an average copy number if the sample is heterogeneous. In some embodiments, the likelihood that a subject with cancer responds to an SF3B1 suppression treatment is increased if the average copy number of SF3B1 in the sample from the subject is at least smaller than the ploidy of the cells in the sample by at least 25%.

In some embodiments, the subjected is treated with an SF3B1 suppression treatment if the likelihood that the subject response is increased.

The present disclosure also provides a method for determining the likelihood that a subject with cancer responds to an SF3B1 suppression treatment, comprising measuring expression level of SF3B1 in a sample from the subject and comparing the measured expression level of SF3B1 in the sample from the subject to the expression level of SF3B1 in a control sample, wherein the likelihood that a subject with cancer responds to an SF3B1 suppression treatment is increased if the expression level of SF3B1 in the sample from the subject is lower than the expression level of SF3B1 in the control sample.

In some embodiments, the cancer is selected from the group consisting of breast cancer, hematopoietic cancer, bladder cancer and kidney cancer. In some embodiments, the cancer is selected from the group consisting of acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, chronic myelogenous leukemia, colon adenocarcinoma, esophageal carcinoma, glioblastoma multiforme, head and neck squamous cell carcinoma, chromophobe renal cell carcinoma, renal clear cell carcinoma, renal papillary cell carcinoma, hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma.

In some embodiments, the sample comprises a cancerous lesion. In some embodiments, the sample comprises circulating tumor cells. In some embodiments, the control sample comprises one or more samples selected from the group consisting of a normal tissue, a tumor known to have the same ploidy as the sample from the subject, and a cell known to have the same ploidy as the sample from the subject.

In some embodiments, the likelihood that a subject with cancer responds to an SF3B1 suppression treatment is increased if the expression level of SF3B1 in the sample from the subject is lower than the expression level of SF3B1 in the control sample by at least 25%.

In some embodiments, the expression level of SF3B1 in the sample from the subject is an mRNA level. In some embodiments, the SF3B1 mRNA level in the sample from the subject is measured by a method comprising quantitative PCR. In some embodiments, the SF3B1 mRNA level in the sample from the subject is measured by a method comprising RNA sequencing. In one embodiment, the RNA sequencing comprises whole-transcriptome sequencing.

In some embodiments, the expression level of SF3B1 in the sample from the subject is a protein level. In one embodiment, the protein level of SF3B1 in the sample from the subject is measured by a method comprising immunohistochemistry. In another embodiment, the protein level of SF3B1 in the sample from the subject is measured by a method comprising enzyme-linked immunosorbent assay (ELISA). In yet another embodiment, the protein level of SF3B1 in the sample from the subject is measured by a method comprising quantitative mass spectrometry.

In some embodiments, the subject is treated with an SF3B1 suppression treatment if the likelihood that the subject response is increased.

In some embodiments, the SF3B1 suppression treatment comprises reducing the amount of SF3B1 mRNA. In one embodiment, reducing the amount of SF3B1 mRNA comprises RNA interference. In a specific embodiment, the RNA interference targets one or more of the sequences selected from SEQ ID NOs 8, 9, 16, 17, 18 and 19. In another embodiment, reducing the amount of SF3B1 mRNA comprises inhibiting one or more transcription cofactors that control SF3B1 transcription. In a specific embodiment, the one or more transcription cofactors that control SF3B1 transcription comprise bromodomain containing 1 (BRD1), bromodomain containing 2 (BRD2), bromodomain containing 3 (BRD3), bromodomain containing 4 (BRD4), or a combination thereof.

In some embodiments, the SF3B1 suppression treatment comprises reducing the amount or activity of SF3B1 protein. In one embodiment, reducing the amount of SF3B1 protein comprises increasing the rate of SF3B1 protein degradation. In a specific embodiment, increasing the rate of SF3B1 protein degradation comprises inhibiting the activity of one or more deubiquitinating enzymes. In another embodiment, reducing the activity of SF3B1 protein comprises inhibiting the interaction between SF3B1 protein and one or more subunits of the SF3B complex. In a specific embodiment, reducing the activity of SF3B1 protein comprises inhibiting the interaction between the SF3B complex and 15S U2 snRNP or 17S U2 snRNP. In another embodiment, reducing the activity of SF3B1 protein comprises inhibiting the incorporation of SF3B1 into 15S U2 snRNP or 17S U2 snRNP.

In some embodiments, the response to an SF3B1 suppression treatment comprises a reduced tumor load, a longer progression-free survival, a longer overall survival, or a combination thereof.

The present disclosure also provides a method for treating a subject with cancer, comprising providing an SF3B1 suppression treatment, thereby treating the cancer in the subject.

In addition, the present disclosure provides a method for treating a subject with cancer, comprising measuring the copy number of SF3B1 in a sample comprising cells from the subject and providing an SF3B1 suppression treatment if the copy number of SF3B1 in the sample from the subject is smaller than the ploidy of the cells in the sample, thereby treating the cancer in the subject.

In some embodiments, the cancer is selected from the group consisting of breast cancer, hematopoietic cancer, bladder cancer and kidney cancer. In some embodiments, the cancer is selected from the group consisting of acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, chronic myelogenous leukemia, colon adenocarcinoma, esophageal carcinoma, glioblastoma multiforme, head and neck squamous cell carcinoma, chromophobe renal cell carcinoma, renal clear cell carcinoma, renal papillary cell carcinoma, hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma.

In some embodiments, the sample comprises a cancerous lesion. In some embodiments, the sample comprises circulating tumor cells.

In some embodiments, measuring the copy number of SF3B1 comprises comparative genomic hybridization (CGH). In some embodiments, measuring the copy number of SF3B1 comprises fluorescence in situ hybridization (FISH). In some embodiments, measuring the copy number of SF3B1 comprises amplifying a genomic sequence comprising at least 20 nucleotides of SF3B1.

In some embodiments, measuring the copy number of SF3B1 comprises DNA sequencing. In one embodiment, DNA sequencing comprises whole-genome sequencing. In another embodiment, DNA sequencing comprises whole-exome sequencing.

In some embodiments, the copy number of SF3B1 in the sample is an average copy number if the sample is heterogeneous. In some embodiments, an SF3B1 suppression treatment is provided to the subject if the average copy number of SF3B1 in the sample from the subject is smaller than the ploidy of the cells in the sample by at least 25%.

The present disclosure also provides a method for treating a subject with cancer, comprising measuring expression level of SF3B1 in a sample from the subject, comparing the measured expression level of SF3B1 in the sample from the subject to the expression level of SF3B1 in a control sample, and providing an SF3B1 suppression treatment if the expression level of SF3B1 in the sample from the subject is lower than the expression level of SF3B1 in the control sample, thereby treating the cancer in the subject.

In some embodiments, the cancer is selected from the group consisting of breast cancer, hematopoietic cancer, bladder cancer and kidney cancer. In some embodiments, the cancer is selected from the group consisting of acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, chronic myelogenous leukemia, colon adenocarcinoma, esophageal carcinoma, glioblastoma multiforme, head and neck squamous cell carcinoma, chromophobe renal cell carcinoma, renal clear cell carcinoma, renal papillary cell carcinoma, hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma.

In some embodiments, the sample comprises a cancerous lesion. In some embodiments, the sample comprises circulating tumor cells. In some embodiments, the control sample comprises one or more samples selected from the group consisting of a normal tissue, a tumor known to have the same ploidy as the sample from the subject, and a cell known to have the same ploidy as the sample from the subject. In some embodiments, an SF3B1 suppression treatment is provided to the subject if the expression level of SF3B1 in the sample from the subject is lower than the expression level of SF3B1 in the control sample by at least 25%.

In some embodiments, the expression level of SF3B1 in the sample from the subject is an mRNA level. In one embodiment, the SF3B1 mRNA level in the sample from the subject is measured by a method comprising quantitative PCR. In another embodiment, the SF3B1 mRNA level in the sample from the subject is measured by a method comprising RNA sequencing. In a specific embodiment, the RNA sequencing comprises whole-transcriptome sequencing.

In some embodiments, the expression level of SF3B1 in the sample from the subject is a protein level. In one embodiment, the protein level of SF3B1 in the sample from the subject is measured by a method comprising immunohistochemistry. In another embodiment, the protein level of SF3B1 in the sample from the subject is measured by a method comprising enzyme-linked immunosorbent assay (ELISA). In yet another embodiment, the protein level of SF3B1 in the sample from the subject is measured by a method comprising quantitative mass spectrometry.

In some embodiments, the SF3B1 suppression treatment comprises reducing the amount of SF3B1 mRNA. In one embodiment, reducing the amount of SF3B1 mRNA comprises RNA interference. In a specific embodiment, the RNA interference targets one or more of the sequences selected from SEQ ID NOs 8, 9, 16, 17, 18 and 19. In another embodiment, reducing the amount of SF3B1 mRNA comprises inhibiting one or more transcription cofactors that control SF3B1 transcription. In a specific embodiment, the one or more transcription cofactors that control SF3B1 transcription comprise bromodomain containing 1 (BRD1), bromodomain containing 2 (BRD2), bromodomain containing 3 (BRD3), bromodomain containing 4 (BRD4), or a combination thereof.

In some embodiments, the SF3B1 suppression treatment comprises reducing the expression or activity of SF3B1 protein. In one embodiment, reducing the amount of SF3B1 protein comprises increasing the rate of SF3B1 protein degradation. In a specific embodiment, increasing the rate of SF3B1 protein degradation comprises inhibiting the activity of one or more deubiquitinating enzymes. In another embodiment, reducing the activity of SF3B1 protein comprises inhibiting the interaction between SF3B1 protein and one or more subunits of the SF3B complex. In another embodiment, reducing the activity of SF3B1 protein comprises inhibiting the interaction between the SF3B complex and 15S U2 snRNP. In yet another embodiment, reducing the activity of SF3B1 protein comprises inhibiting the incorporation of SF3B1 into 15S U2 snRNP or 17S U2 snRNP.

The present disclosure also provides a kit comprising a reagent for reverse transcription of an RNA molecule, two or more primers, wherein a first primer comprises a polynucleotide comprising SEQ ID NO: 24, and a second primer comprises a polynucleotide comprising SEQ ID NO: 25, and a reagent for amplification of a DNA sequence.

In addition, the present disclosure provides a kit comprising an antibody that is capable of binding SF3B1 and a reagent for the detection of the antibody.

### BRIEF DESCRIPTION OF DRAWINGS

FIG. 1A is a series of graphs showing growth of breast cancer cell lines (SF3B1^{neutral}: “Cal 51”, “HMC 1-8”, “Hs578T”, “Cal51 CRISPR^{neutral}” SF3B1^{loss}: “HCC1954”, “BT549”, “T47D”, “Cal51 CRISPR^{frameshift-loss}”) expressing shRNAs targeting lacZ (“shLacZ”) or SF3B1 (“shSF3B1 #3”, “shSF3B1 #4”) measured as changes in CellTiterGlo luminescence relative to one day post-infection.

FIG. 1B is a graph showing quantification of SF3B1 expression from the indicated cell lines (“HMC 1-8”, “Cal-51”, “Hs578T”, “MCF7”, “MCF10A”, “HMEC”, “HCC1954”, “T47D”, “BT549”, “SKBR3”) expressing shRNAs targeting lacZ (“shLacZ,” left bar for each cell line) or SF3B1 (“shSF3B1 #3,” middle bar for each cell line; or “shSF3B1 #4,” right bar for each cell line) by quantitative RT-PCR.

FIG. 1C is a graph showing relative growth of Cal 51 CRISP^{copy-loss} cells and Cal 51 CRISPR^{neutral#2} cells after treatment with siRNAs targeting LacZ (“siLacZ”) or SF3B1 (“siSF3B1 #3”).

FIG. 2A is a graph showing ratio of cells expressing an SF3B1 shRNA coupled with GFP (“shSF3B1-GFP+”) relative to uninfected controls, normalized to the ratio of cells expressing an LacZ shRNA coupled with GFP (“shLacZ-GFP+”) relative to uninfected controls.

FIG. 2B is a series of graphs showing ratio of cells expressing an LacZ shRNA coupled with GFP (“shLacZ-GFP”) or an SF3B1 shRNA coupled with GFP (“shSF3B1#4-GFP”) to uninfected controls in SF3B1^{neutral} and SF3B1^{loss} breast cell lines (SF3B 1^{neutral}: “HMC 1-8”, “Cal51”, “Hs578T”, “MCF10A”; SF3B1^{loss}: “HCC1954”, “T47D”, “BT549”, SKBR3”) and hematopoetic cell lines (SF3B1^{neutral}: “Raji”, “Jurkat”, “HT”; SF3B1^{loss}: “Toledo”, “Hut78”).

FIG. 3A is a graph showing viability of cells (“SF3B1^{neutral}”, “SF3B1^{loss}”) expressing doxycycline (Dox)-activated SF3B1 shRNAs (TR-shSF3B1#3 and TR-shSF3B1#5), cultured in the presence (“+ Dox”) or absence (“− Dox”) of doxycycline, relative to viability three days post Dox treatment.

FIG. 3B is a series of graphs showing quantification of SF3B1 expression without (“−Dox,” left bar for each cell line) or with (“+ Dox,” right bar for each cell line) Dox-induced shSF3B1 (“TR-shSF3B1 #3”, “TR-shSF3B1 #5”) expression by quantitative RT-PCR.

FIG. 3C is a series of graphs showing growth of breast cancer cell lines without (“− Dox”) or with (“+ Dox”) Dox-induced SF3B1 shRNAs (“TR-shSF3B1#3”, “TR-shSF3B1#5”) expression, measured as changes in CellTiterGlo luminescence relative to day 1 of Dox treatment.

FIG. 4A is a series of graphs showing cell cycle distribution in SF3B1^{neutral} and SF3B1^{loss} cells incubated for four days without (“− Dox,” left bar for each cell line) or with (“+ Dox,” right bar for each cell line) Dox-induced expression of shSF3B1.

FIG. 4B is a series of graphs showing the fraction of apoptotic cells five days after incubation without (“− Dox,” left bar for each cell line) or with (“+ Dox,” right bar for each cell line) Dox-induced expression of shSF3B1, as determined by Annexin V/prodium iodide flow cytometry.

FIG. 4C is a graph showing viability of cells expressing shRNAs targeting LacZ (“shLacZ”) or SF3B1 (“TR-shSF3B1”: an average of replicates performed using independent shRNAs “shSF3B1 #3” and “shSF3B1 #4”), measured as fractions of cells excluding propidium iodide, relative to viability of these cells four days post infection.

FIG. 5A is a series of graphs showing quantification of GFP fluorescence from cells expressing SF3B1-IRES-GFP without (“−dox,” left four bars in each panel) or with (“+dox,” right four bars in each panel) Dox-induced SF3B1 shRNA expression.

FIG. 5B is a graph showing ratio of cells expressing SF3B1-GFP (“+SF3B1”) relative to uninfected (“control”) SF3B1^{neutral} cells (“Cal51”) and SF3B1^{loss} cells (“HCC1954”).

FIG. 5C is a graph showing ratio of cells expressing SF3B1-GFP (“+SF3B1”) relative to uninfected SF3B1^{neutral} cells (“Cal51”) and SF3B1^{loss} cells (“HCC1954”) expressing an shRNA targeting SF3B1 (“shSF3B1”).

FIG. 6A is an immunoblot of SF3B1 from HCC1954 cells expressing LacZ or SF3B1.

FIG. 6B is a graph showing growth of SF3B1^{loss} cells expressing LacZ or SF3B1 upon Dox-induced expression of SF3B1 shRNA (“TR shSF3B1#5”), measured as changes in CellTiter-Glo luminescence.

FIG. 7A is a graph showing SF3B1 expression from 777 TCGA breast adenocarcinomas segregated by SF3B1 copy number. Whiskers represent min/max values and boxes represent upper and lower quartile ranges. Width of plots represents relative sample density.

FIG. 7B is a graph showing SF3B1 expression from 974 cell lines from the Cancer Cell Line Encyclopedia (CCLE) classified by SF3B1 copy-number status. Boxes represent the upper and lower quartiles, and whiskers represent the 5-95th percentiles. *p<0.0001.

FIG. 8A is a graph showing SF3B1 expression in SF3B1^{neutral} and SF3B1^{loss} breast cancer cell lines measured by quantitative RT-PCR. Data points represent individual cell lines, and horizontal lines indicate means.

FIG. 8B is a graph showing SF3B1 mRNA expression from control cells and those with CRISPR-induced copy-loss.

FIG. 9A is an immunoblot showing SF3B1 protein levels in breast cancer cell lines (SF3B1^{neutral}: “Cal51”, “Hs578T”, “MCF7”; SF3B1^{loss}; “BT549”, “HCC1954”, “ZR-75-30”).

FIG. 9B is an immunoblot showing SF3B1 expression from control Cal51 cells (“control-1” and “control-2”), Cal51 cells containing a frameshift mutation inactivating one SF3B1 allele (“Loss-1”), and Cal51 cells having deletion of one copy of the SF3B1 locus (“Loss-2”). The Loss-1 and Loss-2 cells were generated by CRISPR technology.

FIG. 9C is a scatterplot of SF3B1 mRNA and protein expression relative to diploid cell line Cal51 after normalization to actin in a panel of breast cancer cell lines (p=0.0018, R²=0.772, regression line slope=0.789).

FIG. 10 is an immunoblot of SF3B1^{neutral} cells (“Cal51”, “Hs578T”, “MCF7”) and SF3B1^{loss} cells (“BT549”, “HCC1954”) without and with Dox-induced expression of shSF3B1#5.

FIG. 11 is a graph showing differences in proliferation (measured by CellTiter-Glo; red=high, blue=low) against relative level of SF3B1 expression (assessed by qPCR; y-axis) in F3B1^{neutral} and SF3B1^{loss} cells expressing either shLacZ (origins of arrows) or shSF3B1 (ends of arrows). Origins with multiple arrows represent cell lines subject to more than one SF3B1 shRNA. Each data point represents the mean from at least two replicate experiments. The dashed line represents the estimated minimum threshold of SF3B1 expression required for survival.

FIG. 12 is a diagram of U2 snRNP assembly.

FIG. 13A is a graph showing sedimentation of mass standards in 10-30% glycerol gradients.

FIG. 13B is a graph showing elution profiles of mass standards in gel filtration chromatography columns.

FIG. 14A is an immunoblot showing input for glycerol gradient fractionation of native whole-cell lysates of breast cancer cell lines (SF3B1^{neutral}: “Cal51”, “Hs578T”; SF3B1^{loss}; “BT549”, “HCC1954”).

FIG. 14B is an immunoblot showing fractions from glycerol gradient fractionation of native whole-cell lysates of breast cancer cell lines (SF3B1^{neutral}: “Cal51”, “Hs578T”; SF3B1^{loss}: “BT549”, “HCC1954”).

FIG. 15A is an immunoblot showing input for glycerol gradient fractionation of native whole-cell lysates of isogenic cells generated by CRISPR (“neutral#1”, “frameshift-loss”).

FIG. 15B is an immunoblot showing fractions from glycerol gradient fractionation of native whole-cell lysates of isogenic cells generated by CRISPR (“neutral#1”, “frameshift-loss”).

FIG. 15C is a graph showing quantification of SF3B1 from immunoblots of samples from glycerol gradient fractions 3-8, relative to fraction 3 (n=3 for each group).

FIG. 16A is an immunoblot showing the amount of SF3B1 in pooled glycerol gradient fractions (“4-6”, “12-14”, “25”) of breast cancer cell lines (SF3B1^{neutral}: “Cal51”, “Hs578T”; SF3B1^{loss}: “BT549”, “HCC1954”) in serial dilution.

FIG. 16B is an immunoblot of indicated gel filtration fractions. GAPDH and SNRPB2 represent markers for complexes <700 kDa and spliceosome precursors respectively.

FIG. 17A is (left) an immunoblot showing SF3B1 Native PAGE of pooled glycerol gradient fractions 4-6 and (right) a denaturing silver stain of total protein from the same pooled fractions as loading control.

FIG. 17B is an immunoblot after SF3B1 immunoprecipitation from pooled glycerol gradient fractions 4-6.

FIG. 18 is an immunoblot after SF3B1 immunoprecipitation from pooled glycerol gradient fractions 24-25.

FIG. 19A is a quantification of U2 snRNA expression in three SF3B1^{neutral}and three SF3B1^{loss} breast cancer cell lines quantitative RT-PCR. ns=not significant.

FIG. 19B is a representative radiologic image of a native agarose gel of U2 snRNP complexes visualized with radiolabeled 2′ O-methyl oligonucleotides complementary to the U2 snRNA. Nuclear extracts were generated from control Cal51 cells (“control-1” and “control-2”), Cal51 cells containing a frameshift mutation inactivating one SF3B1 allele (“Loss-1”), and Cal51 cells having deletion of one copy of the SF3B1 locus (“Loss-2”). HeLa cell nuclear extracts (“Hela NE”) in the absence of presence of ATP were used as controls.

FIG. 19C is a graph showing densitometric quantification of 17S U2 snRNP bands in FIG. 19B, presented as fold change relative to the control Cal51 cells. Data are from three replicate experiments.

FIG. 20 is a diagram showing a model for changes to U2 snRNP assembly associated with SF3B1 copy-loss.

FIG. 21A is an immunoblot showing input for glycerol gradient fractionation of lysates of breast cancer cell lines (SF3B1^{neutral}: “Cal51”, “Hs578T”; SF3B1^{loss}: “BT549”, “HCC1954”) without and with Dox-induced SF3B1 suppression.

FIG. 21B is an immunoblot showing fraction 25 (protein complexes >650 kDa) from glycerol gradient fractionation of lysates of breast cancer cell lines (SF3B1^{neutral}: “Cal51”, “Hs578T”; SF3B1^{loss}: “BT549”, “HCC1954”) without and with Dox-induced F3B1 suppression.

FIG. 22A is an immunoblot showing input for gel filtration chromatography of lysates of breast cancer cell lines (SF3B1^{neutral}: “Cal51”; SF3B1^{loss}: “HCC1954”) without and with Dox-induced SF3B1 suppression.

FIG. 22B is an immunoblot showing fractions 18-26 (protein complexes >650 kDa) from gel filtration chromatography of lysates of breast cancer cell lines (SF3B1^{neutral}: “Cal51”; SF3B1^{loss}: “HCC1954”) without and with Dox-induced SF3B1 suppression.

FIG. 22C is a silver stain of gel filtration inputs for FIG. 22B.

FIG. 23 is a graph showing quantification of U2 snRNA expression in breast cancer cell lines (SF3B1^{neutral:} “Cal51”, “Hs578T”; SF3B1^{loss}: “BT549”, “HCC1954”) without (“− Dox,” left bar for each cell line) or with (“+ Dox,” right bar for each cell line) Dox-induced F3B1 suppression by quantitative RT-PCR.

FIG. 24 is an immunoblot of pooled glycerol gradient fractions 4-6 (protein complexes 150-450 kDa) from lysates of breast cancer cell lines (SF3B1^{neutral}: “Cal51”, “Hs578T”; SF3B1^{loss}: “BT549”, “HCC1954”) without and with Dox-induced SF3B1 suppression.

FIG. 25 is an immunoblot of glycerol gradient fractions 3-6 from SF3B1^{neutral} cells (“Cal51”, “Hs578T”) and SF3B1^{loss} cells (“HCC1954”) without and with Dox-induced SF3B1 suppression.

FIG. 26A is a series of graphs showing drug sensitivity curves for indicated splicing inhibitors (“NSC-95397”, “pladienolide B”, “spliceostatin A”) in cells (“Hs578T”, “Cal51”) without and with SF3B1 suppression.

FIG. 26B is an immunoblot from cells used in FIG. 26A.

FIG. 27A is a graph showing relative levels of SF3B1 expression (assessed by qPCR; y-axis) in SF3B1^{neutral} (left) or SF3B1^{loss} (right) cells without doxycycline (origins of arrows) or with doxycycline (ends of arrows), wherein the cells were used in the RNA sequencing analysis as described in FIG. 28. Origins with multiple arrows represent cell lines subject to more than one SF3B1 shRNA. Each data point represents the mean from at least two replicate experiments.

FIG. 27B is a graph showing relative levels of SF3B1 expression in CRISPR^{neutral#1} and CRISPR^{frameshift-loss} presented as described in FIG. 27A.

FIG. 28A is a graph showing statistical significance of intron retention across all exon-intron junctions (dots) in SF3B1^{neutral} (left) and SF3B1^{loss} cells (right) after SF3B1 suppression. The horizontal dashed line represents the significance threshold (q<0.01) and the vertical dashed line segregates intron-exon junctions more likely to be altered in SF3B1^{neutral} or SF3B1^{loss} cells.

FIG. 28B is a graph showing qPCR for a single intron within the indicated genes (“AARS,” “CalR,” “DNAJB1,” “MKNK2,” “MYH9,” “RPS8,” and “RPS18”) without (“− Dox,” left bar for each cell line) or with (“+ Dox,” right bar for each cell line) shSF3B1 induction by doxycycline (SF3B1^{neutral} n=3, SF3B1^{loss} n=3, averaged from TR-shSF3B1#3 and TRshSF3B1#5.

FIG. 28C is a graph showing statistical significance of alternative 3′ splice site selection across 3′ splice junctions (dots) in SF3B 1^{neutral} (left) and SF3B1^{loss} cells (right) after SF3B1 suppression. The horizontal dashed line represents the significance threshold (q<0.01) and the vertical dashed line segregates 3′ splicing more likely to be altered in SF3B1^{neutral} or SF3B1^{loss} cells.

FIG. 29A is a diagram showing a method for measuring intron retention. Arrowheads indicate primer locations used in FIG. 29B. Numbers represent exons of indicated genes.

FIG. 29B is an image of DNA electrophoresis following RT-PCR for RPS18 and CALR in cells (SF3B1^{neutral}: “Cal51”, “Hs578T”; SF3B1^{loss}: “HCC1954”, “BT549”) without and with shSF3B1 induction by doxycycline. Arrows indicate PCR products corresponding to retained introns.

FIG. 30A is an image of DNA electrophoresis following a representative RT-PCR from SF3B1^{neutral} (“Cal51”) and SF3B1^{loss} (“HCC 1954”) cells after SF3B1 knockdown. “c” represents LacZ control hairpins, “sh” represents shSF3B1#4 hairpins. Arrows represent product sizes for MCL-L and MCL-S.

FIG. 30B is a graph showing densitometric quantification of the ratio of MCL1-S:MCL1-L in cells expressing shSF3B1 (right bar for SF3B1^{neutral} cells and for SF3B1^{loss} cells) relative to shLacZ-expressing controls (left bar for SF3B1^{neutral} cells and for SF3B1^{loss} cells) (mean+/−SD from three biological replicates of at least 3 cell lines per group).

FIG. 31A is a series of immunofluorescent images of nuclear speckles by anti-SC35 (SRSF2) staining. Scale bar=5 uM.

FIG. 31B is a series of graphs showing quantification of number of nuclear speckles (upper) and speckle area (lower) per cell across at least 100 nuclei in cells without (“− Dox,” left bar for each cell line) or with (“+ Dox,” right bar for each cell line) shSF3B1 induction by doxycycline.

FIG. 32 is a diagram depicting a number of differentially expressed genes upon SF3B1 suppression (q<0.1) and the number of enriched KEGG pathways amongst indicated gene set (q<0.05).

FIG. 33 is a heatmap of False Discovery Rate q-values indicating the significance of associations between copy numbers of SF3b complex members (rows) and sensitivity of those cells to suppression of SF3b complex members by shRNA (columns).

FIG. 34 is a graph showing luminescent quantification of xenograft growth from CRISPR^{neutral#1} and CRISPR^{frameshift-loss} tumors without doxycycline administration to the mice (n=4).

FIG. 35A is a graph showing luminescent quantification of xenograft growth from CRISPR^{neutral#1} and CRISPR^{frameshift-loss} tumors with doxycycline administration to the mice (n=17).

FIG. 35B is a series of photographs of animals overlaid with heat maps from bioluminescent tumor detection. Dashed circle represents region where established tumor was detected prior to doxycycline treatment.

FIG. 35C is a series of representative Ki67 immunohistochemistry images of xenografts.

FIG. 35D is a graph showing quantification of Ki67+ cells from xenografts in FIG. 35C using CellProfiler. The bars on the left represent the ratio of Ki67+ cells in the xenograft from CRISPR^{neutral#1} tumors, and the bars on the right represent the ratio of Ki67+ cells in the xenograft from CRISPR^{frameshift-loss} tumors. A minimum of 2,440 nuclei were scored for each tumor, >=3 tumors per group.

FIG. 35E is a graph showing quantification of SF3B1 expression from xenograft tumors without (“− Dox,” bars on the left for each cell line) or with (“+ Dox,” bars on the right for each cell line) doxycycline-induced shSF3B1 expression (n>=4 for each group) by quantitative RT-PCR.

FIG. 36A is a graph showing growth of established tumors for Cal51 xenografts without doxycycline (“−Dox”, n=13) or with doxycycline (“+Dox”, n=12) using TR-shSF3B1 #3.

FIG. 36B is a graph showing growth of established tumors for HCC1954 xenografts without doxycycline (“−Dox”, n=13) or with doxycycline (“+Dox”, n=12) using TR-shSF3B1 #3. ***p≤0.001.

FIG. 37 is an immunoblot showing SF3B1 expression in Cal51 cells treated with de-ubiquitinase (DUB) inhibitors (“PR-619”, “b-AP15”, “SJB3-019A”) for 4 or 24 hours. For all figures, *p<0.05, **p<0.01, ***p<0.001 unless otherwise indicated, and error bars represent +/−standard deviation.

### DETAILED DESCRIPTION

The present disclosure identifies SF3B1 as a CYCLOPS gene, wherein the copy-number of SF3B1 is associated with the dependency of cell growth on the remaining expression of SF3B1. Cancer cells that have lost at least one copy of SF3B1 from the genome are more sensitive to SF3B1 suppression than cells having the normal complement of SF3B1 copies. While suppression of SF3B1 in SF3B1 copy-loss cells can reduce the amount of SF3B1 below the threshold level for maintaining cell proliferation, two copies of SF3B1 in a normal cell's genome provide an excess reservoir to render the cells insensitive to SF3B1 suppression. SF3B1 suppression in SF3B1 copy-loss cells substantially decreases levels of U2 snRNP precursor and leads to spicing defects. Tumors from SF3B1 copy-loss genetic backgrounds regress or grow more slowly when SF3B1 is suppressed in a xenograft model in mice.

In certain embodiments, the copy number SF3B1 is measured in a tumor for diagnosis and therapy selection. In one embodiment, a tumor with SF3B1 copy loss is predicted to be sensitive to an SF3B1 suppression treatment. At the same time, non-tumorous cells with both copies of SF3B1 intact are more resistant to SF3B1 suppression, thereby providing a therapeutic window to selectively suppress the tumor. In another embodiment, an SF3B1 suppression treatment is not selected for treating a tumor without SF3B1 copy loss because these tumor cells have a larger reservoir of SF3B1 and are thus not as sensitive to SF3B1 suppression.

In certain embodiments, the expression level of SF3B1 is measured in a tumor for diagnosis and therapy selection. In one embodiment, a tumor with lower SF3B1 expression level is predicted to be sensitive to an SF3B1 suppression treatment. At the same time, non-tumorous cells, to which the SF3B1 expression level in the tumor may be compared, are more resistant to SF3B1 suppression, thereby providing a therapeutic window to selectively suppress the tumor. The differential expression of SF3B1 in tumor cells may be caused by various reasons, such as epigenetic alteration, genetic alteration of one or more factors that regulates SF3B1 expression, alteration of the expression of one or more factors that regulates SF3B1 expression. In another embodiment, an SF3B1 suppression treatment is not selected for treating a tumor without lower SF3B1 expression level because these tumor cells have a larger reservoir of SF3B1 and are thus not as sensitive to SF3B1 suppression.

In certain embodiments, SF3B1 suppression is provided as a treatment for cancer. In one embodiment, this treatment is provided if the cancer is diagnosed to have an SF3B1 copy loss. In another embodiment, this treatment is provided if the cancer is diagnosed to have a lower SF3B1 expression.

In certain embodiments, SF3B1 suppression is provided by reducing the effective amount of SF3B1 mRNA or protein. In one embodiment, the effective amount of SF3B1 mRNA is the amount of SF3B1 mRNA or a functional form thereof. A functional form of SF3B1 mRNA encompasses, but is not limited to, mature SF3B1 mRNA, SF3B1 mRNA under active translation, SF3B1 mRNA in the cytosol, SF3B1 mRNA in a polysome, SF3B1 mRNA not bound by an siRNA, shRNA or microRNA. In another embodiment, the effective amount of SF3B1 protein is the amount of SF3B1 protein or a functional form thereof. A functional form of SF3B1 protein encompasses, but is not limited to, SF3B1 protein in a cell nucleus, SF3B1 protein in a nuclear speckle, SF3B1 protein in an SF3B complex, SF3B1 protein in a U2 snRNP, SF3B1 protein having a post-translational modification that correlates with the activity of a complex comprising SF3B1.

As used herein, “a subject” encompasses, but is not limited to, a mammal, e.g. a human, a domestic animal or a livestock including a cat, a dog, a cattle and a horse.

“An SF3B1 suppression treatment” encompasses, but is not limited to, (1) a treatment that reduces the amount of SF3B1 mRNA or a functional form thereof by at least 10%, 20%, 30%, 40%, 50%, 60% or 70%, (2) a treatment that reduces the amount of SF3B1 protein or a functional form thereof by at least 10%, 20%, 30%, 40%, 50%, 60% or 70%, and (3) a treatment that reduces the activity of a complex comprising SF3B1 by at least 10%, 20%, 30%, 40%, 50%, 60% or 70%. As used herein, a functional form of SF3B1 mRNA encompasses, but is not limited to, mature SF3B1 mRNA, SF3B1 mRNA under active translation, SF3B1 mRNA in the cytosol, SF3B1 mRNA in a polysome, SF3B1 mRNA not bound by an siRNA, shRNA or microRNA. A functional form of SF3B1 protein encompasses, but is not limited to, SF3B1 protein in a cell nucleus, SF3B1 protein in a nuclear speckle, SF3B1 protein in an SF3B complex, SF3B1 protein in a U2 snRNP, SF3B1 protein having a post-translational modification that correlates with the activity of a complex comprising SF3B1. A complex comprising SF3B1 encompasses, but is not limited to, monomeric SF3B1, an SF3B complex, a 15S U2 snRNP complex, a 17S U2 snRNP complex, and polycomb repressor complex.

“Copy number of SF3B1” encompasses, but is not limited to, the numbers of copies of SF3B1 in the genome of a cell, tissue, or organ. In most somatic cells of a diploid subject, the copy number of SF3B1 is 2.

“Measuring the copy number of SF3B1” encompasses, but is not limited to, measuring the copy number of SF3B1 by a laboratory method, and obtaining data from an agency that examines the copy number of SF3B1. The laboratory method of measurement encompasses, but is not limited to, comparative genomic hybridization (CGH), fluorescence in situ hybridization (FISH), DNA amplification and DNA sequencing.

“A sample comprising cells” encompasses, but is not limited to, a sample comprising cells from a tumor lesion, a sample from a cancer draining lymph node, a body fluid such as blood, serum, plasma, urine, semen, lymph, and peritoneal fluid.

“The ploidy” of the cells in the sample refers to the number of sets of chromosomes of the cells in the sample. In some embodiments, the cells have aneuploidy and “the ploidy” refers to the number of sets of at least 50%, 60%, 70%, 80%, 90% or 95% of all chromosomes in the organism from which the sample is obtained. In some embodiments, the ploidy is measured by a cytogenetic method, such as karyotyping and fluorescence in situ hybridization (FISH). In one embodiment, the ploidy of the cells is 2.

“Smaller than the ploidy of the cells” encompasses, but is not limited to, at least 1%, 5%, 10%, 20%, 30%, 40% or 50% smaller than the ploidy of the cells.

“Breast cancer” is a tumor and/or a cancer that originate from a cell of the breast. Common types of breast cancer include but are not limited to ductal carcinoma, lobular carcinoma, tubular carcinoma, medullary carcinoma, mucinous carcinoma, papillary carcinoma, cribriform carcinoma, sarcoma, inflammatory breast cancer, male breast cancer, Paget's disease of the breast, phyllodes tumor. “Breast cancer” herein also includes primary, recurrent and metastatic breast cancer.

“Hematopoietic cancer” is a tumor and/or cancer that originate in a hematopoietic tissue. Hematopoietic tissues include but are not limited to lymphoid and myeloid tissues. Examples of lymphoid cancers include acute lymphocytic leukemia (ALL), Hodgkin's lymphoma, and non-Hodgkin's lymphoma. ALL includes but is not limited to T cell ALL, pro-B cell ALL, pre-B cell ALL, and naive B cell ALL. Non-Hodgkin's lymphoma includes but is not limited to follicular lymphoma (FL), chronic lymphocytic leukemia (CLL), Burkitt's Lymphoma, diffuse large B cell lymphoma (DLBCL), and mantle cell lymphoma (MCL). Examples of myeloid cancers include acute myeloid leukemias (AML), acute monocytic leukemia (AMoL), myelodysplastic syndromes (MDS), chronic myeloid leukemia (CML) and other myeloproliferative diseases (e.g., osteomyelofibrosis, polycythemia vera and essential thrombocythemia). “Hematopoietic cancer” herein also includes primary, recurrent and metastatic hematopoietic cancer.

“Bladder cancer” is a tumor and/or cancer that originate from a bladder cell. It encompasses, but is not limited to, superficial bladder cancer (often urothelial carcinoma), muscle invasive bladder cancer, small cell carcinoma, squamous carcinoma, adenocarcinoma, and leiomyosarcoma. “Bladder cancer” herein also includes primary, recurrent and metastatic bladder cancer.

“Kidney cancer” is a tumor and/or cancer that originate in the kidney. It encompasses, but is not limited to, renal cell carcinoma, transitional cell cancer, nephroblastoma, renal sarcoma, and benign kidney tumors (e.g. renal adenoma, oncocytoma, angiomyolipoma).

“A cancerous lesion” encompasses, but is not limited to, a tissue, organ or structure where cancer locates. It may be at a primary site or a metastatic site.

“Circulating tumor cells” encompass, but are not limited to, cells with a tumor origin in the circulating blood stream. In certain embodiments, the circulating tumor cells are enriched from the blood.

“Comparative genomic hybridization (CGH)” encompasses, but is not limited to, a cytogenetic method for analyzing copy number variations relative to ploidy levels in a DNA sample by hybridizing sample DNA with probe DNA. Wherein the probe DNA molecules are provided in an array, CGH can be referred to as array CGH.

“Fluorescence in situ hybridization (FISH)” encompasses, but is not limited to, a cytogenetic technique for detecting and locating a DNA sequence of interest on a chromosome, wherein at least one probe conjugated to a fluorescent moiety is hybridized to the DNA sequence of interest. A FISH probe herein for measuring the copy number of SF3B1 encompasses, but is not limited to, a nucleic acid conjugated to a fluorescent moiety capable of hybridizing to SEQ ID NO. 1 or a genomic sequence within 5 kb, 10 kb, 20 kb, 50 kb or 100 kb away from either terminus of the genomic location of SEQ ID NO. 1.

“A genomic sequence comprising at least 20 nucleotides of SF3B1” encompasses, but is not limited to, (1) a polynucleotide comprising at least 20 nucleotides from SEQ ID NO. 1 or a genomic sequence within 5 kb, 10 kb, 20 kb, 50 kb or 100 kb away from either terminus of the genomic location of SEQ ID NO. 1, and (2) a sequence complementary to the polynucleotide of (1).

“Amplifying a genomic sequence” encompasses, but is not limited to, amplifying a target genomic sequence by polymerase chain reaction (PCR). In one aspect, a probe conjugated to a detectable moiety that hybridizes to the amplified sequence is included in the PCR reaction for quantification of the target genomic sequence.

“Whole-exome sequencing” encompasses, but is not limited to, sequencing of all protein coding genes in a genome. In one aspect, quantitative information is obtained from the sequencing.

“The sample is heterogeneous” means the sample contains cells that are not identical in genetic, epigenetic and/or gene expression status. In one aspect, the sample contains cells from different cell types or origins. In one aspect, the sample contains tumor cells and non-tumor cells. In one aspect, the sample comprises tumor cells wherein some, but not all, of the tumor cells harbor a mutation (e.g. a copy number variation, a transcriptional or epigenetic alteration).

“Average copy number” of a gene in a heterogeneous sample is the average number of copies of the gene. In one aspect, it is measured by CGH directly. In another aspect, copy numbers of the gene in individual cells are measured by FISH and the average copy number is calculated therefrom.

“Expression level of SF3B1” encompasses, but is not limited to, the amount of SF3B1 mRNA or a functional form thereof, and the amount of SF3B1 protein or a functional form thereof. A functional form of SF3B1 mRNA encompasses, but is not limited to, mature SF3B1 mRNA, SF3B1 mRNA under active translation, SF3B1 mRNA in the cytosol, SF3B1 mRNA in a polysome, SF3B1 mRNA not bound by an siRNA, shRNA or microRNA. A functional form of SF3B1 protein encompasses, but is not limited to, SF3B1 protein in a cell nucleus, SF3B1 protein in a nuclear speckle, SF3B1 protein in an SF3B complex, SF3B1 protein in a U2 snRNP, SF3B1 protein having a post-translational modification that correlates with the activity of a complex comprising SF3B1.

“A control sample” encompasses, but is not limited to, a normal tissue, a tumor known to have 2 copies of SF3B1 genomic DNA, and a cell known to have 2 copies of SF3B1 genomic DNA, wherein the cell may be a primary cell or an immortalized cell.

“The expression level of SF3B1 in the sample from the subject is lower than the expression level of SF3B1 in the control sample” means the amount of SF3B1 mRNA or a functional form thereof, or an SF3B1 protein or a functional form thereof is lower than 95%, 90%, 80%, 70%, 60%, 50%, 40%, or 30% of the corresponding expression level of SF3B1 in the control sample.

“RNA sequencing” encompasses, but is not limited to, sequencing of at least one RNA molecule, and sequencing of at least one nucleic acid molecule that is synthesized to be complementary to at least one RNA molecule, wherein the at least one nucleic acid molecule includes, but is not limited to, at least one DNA molecule. In one aspect, quantitative information is obtained from the sequencing.

“Whole-transcriptome sequencing” encompasses, but is not limited to, RNA sequencing of all detectable RNA molecules, all detectable messenger RNA molecules, all detectable pre-messenger RNA molecules, all detectable small RNA molecules, and a combination thereof.

“Immunohistochemistry” encompasses, but is not limited to, a process of detecting an antigen (e.g. SF3B1) in cells of a tissue section using an antibody capable of binding to the antigen (e.g. SF3B1). As used in this disclosure, an antibody that is capable of binding to SF3B1 encompasses, but is not limited to, an anti-SF3B1 antiserum, an anti-SF3B1 polyclonal antibody, an anti-SF3B1 monoclonal antibody, an antigen-binding fragment of an anti-SF3B1 antibody, a protein comprising a heavy chain variable domain that binds to SF3B1, a protein comprising a light chain variable domain that binds to SF3B1, and a protein that binds to SF3B1 with a Kd lower than about 1×10⁻⁶M (e.g., 1×10⁻⁷ M, 1×10⁻⁸M, 1×10⁻⁹ M, 1×10⁻¹⁰ M, 1×10⁻¹¹ M, 1×10⁻¹² M, or lower).

“Quantitative mass spectrometry” is an analytical chemistry technique for determining the amount of one or more proteins in a sample by mass spectrometry. One or more processes for protein purification, enrichment and/or separation may precede the mass spectrometry step.

“RNA interference” encompasses, but is not limited to, reducing the amount or activity of a first messenger RNA (mRNA) molecule by introducing a second RNA molecule that hybridizes to the first mRNA, or by introducing a DNA molecule that is transcribed and/or processed into the second RNA. The activity of a messenger RNA hereby refers to the efficiency that the messenger RNA is translated into a polypeptide. Commonly used RNA interference technologies include, but are not limited to, microRNA, small interfering RNA (siRNA) and small hairpin RNA (shRNA).

“Transcription cofactors that control SF3B1 transcription” encompasses, but is not limited to, histone binding proteins such as BET bromodomain proteins (BRD1-4), and histone modifying enzymes such as histone deacetylases, histone methylases and histone kinases. The transcription cofactor can be inhibited, e.g., by suppressing its expression and/or activity, thereby reducing the activity of the transcription cofactor and SF3B1 transcription.

“SF3B1 protein degradation” encompasses, but is not limited to, proteolysis of SF3B1 and depletion of SF3B1 from a cellular compartment where the protein is assembled into a complex or exerts its function. Proteolysis of SF3B1 includes but is not limited to direct protein cleavage by a protease, ubiquitin-mediated proteolysis by the proteasome, and autophagy-mediated proteolysis in the lysosome (e.g. through macro-autophagy, through chaperone-mediated autophagy). Depletion of SF3B1 from a cellular compartment includes but is not limited to translocation of SF3B1 to a different cellular or extracellular compartment.

“Inhibiting the activity of one or more deubiquitinating enzymes” encompasses, but is not limited to, reducing the amount and/or activity of at least one deubiquitinating enzymes, which leads to increased ubiquitination of an SF3B1 protein and increased degradation of the protein.

“Reducing the activity of SF3B1 protein” encompasses, but is not limited to, reducing the activity of a complex comprising SF3B1 by at least 10%, 20%, 30%, 40%, 50%, 60% or 70%.

“Subunits of the SF3B complex” encompass, but are not limited to, SF3B1, SF3B2, SF3B3, SF3B4, SF3B5 (SF3B10), SF3B14 (SF3B14a), PHFSA (SF3B14b), DDX42, and a pre-mRNA.

“Inhibiting the interaction between SF3B1 protein and one or more subunits of the SF3B complex” encompasses, but is not limited to, inhibiting the physical binding between SF3B1 protein and one or more subunits of the SF3B1 complex, and inhibiting the expression of one or more subunits of the SF3B1 complex. In one aspect, the physical binding is inhibited by a chemical compound, a peptide, a modified peptide or a protein that interferes with protein-protein binding in the SF3B complex. In another aspect, the physical binding is inhibited by an RNA molecule, a modified RNA molecule, a chemical compound (e.g. one that mimicks the structure of an RNA molecule), a peptide, a modified peptide or a protein that interferes with protein-RNA binding in the SF3B complex. In yet another aspect, the expression of one or more subunits of the SF3B1 complex is inhibited at the transcriptional, translational, or post-translational (e.g. protein modification, protein degradation) level.

“Inhibiting the interaction between the SF3B complex and 15S U2 snRNP” can be achieved by preventing the protein:protein binding interactions between SF3B complex members and 15S U2 snRNP during U2 snRNP assembly. It also can be achieved by preventing the protein:RNA binding interactions between SF3b complex and the U2 snRNA (another component of the U2 snRNP). Agents that inhibit the interactions include, but are not limited to, small molecule compounds, peptides, nucleic acids, and a combination or conjugate thereof.

“Tumor load” encompasses, but is not limited to, the number of cancer cells, the size of a tumor, and/or the amount of cancer in the subject. The tumor load may be determined by measuring the tumor size or by measuring a tumor marker or antigen.

“Progression-free survival” encompasses, but is not limited to, the length of time during and after the treatment of a disease, such as cancer, that a patient lives with the disease but it does not get worse.

“Overall survival” encompasses, but is not limited to, the length of time from either the date of diagnosis or the start of treatment for a disease, such as cancer, that patients diagnosed with the disease are still alive.

Furthermore, in accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

The following examples are provided to further elucidate the advantages and features of the present application, but are not intended to limit the scope of the application. The examples are for illustrative purposes only.

### EXAMPLES

### Example 1

### SF3B1 was Frequently Lost in Cancer

The TCGA PanCan dataset was analyzed to search for cancer-associated genes. It was found that SF3B1 was partially lost in 11% of the 10,570 cancers from more than 30 tumor types. Losses were most frequent in invasive breast adenocarcinoma (20%), urothelial bladder carcinoma (32%) and chromophobe kidney carcinoma (71%). Examples of types of cancer wherein an SF3B1 copy loss was identified included, acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, chronic myelogenous leukemia, colon adenocarcinoma, esophageal carcinoma, glioblastoma multiforme, head and neck squamous cell carcinoma, chromophobe renal cell carcinoma, renal clear cell carcinoma, renal papillary cell carcinoma, hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma. Genomic deletions of SF3B1 usually affected most of the chromosome arm (81% of losses) and were never homozygous (0/10,570 cancers), consistent with characterization of SF3B1 as an essential gene. In contrast, 85% of genes were homozygously deleted at least once. Similarly, analysis of copy number alterations from 1042 cancer cell lines in the CCLE indicated 24% of cell lines harbor hemizygous SF3B1 deletion, including 16/61 (26%) of breast cancer cell lines, but never homozygous loss (0/1042 cell lines).

### Example 2

### SF3B1 Suppression Led to Growth Defect of Cells with SF3B1 Copy-Loss

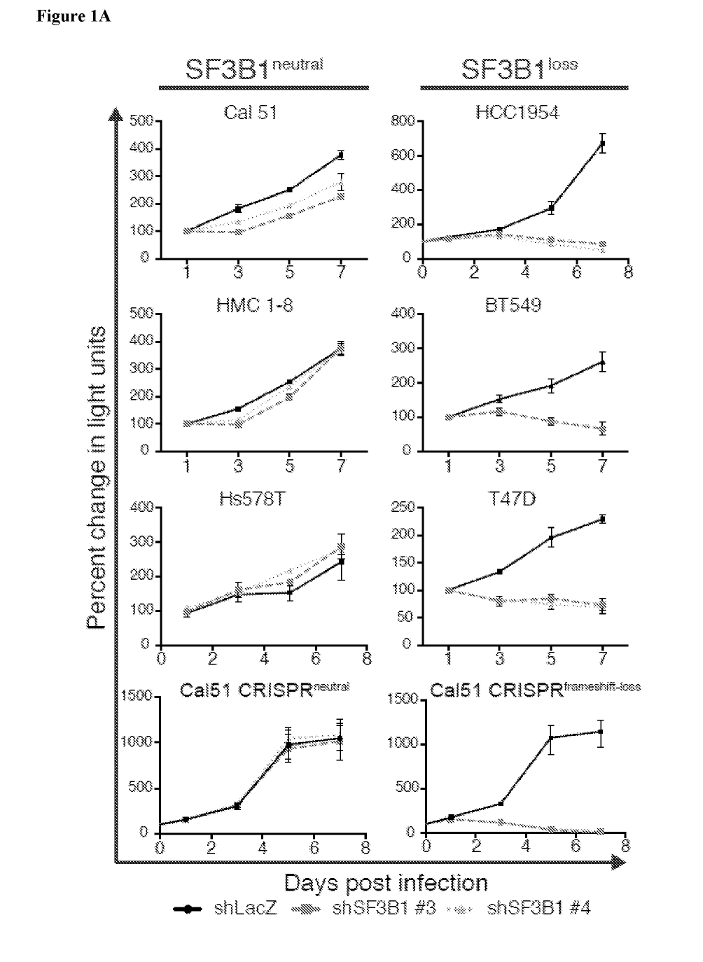

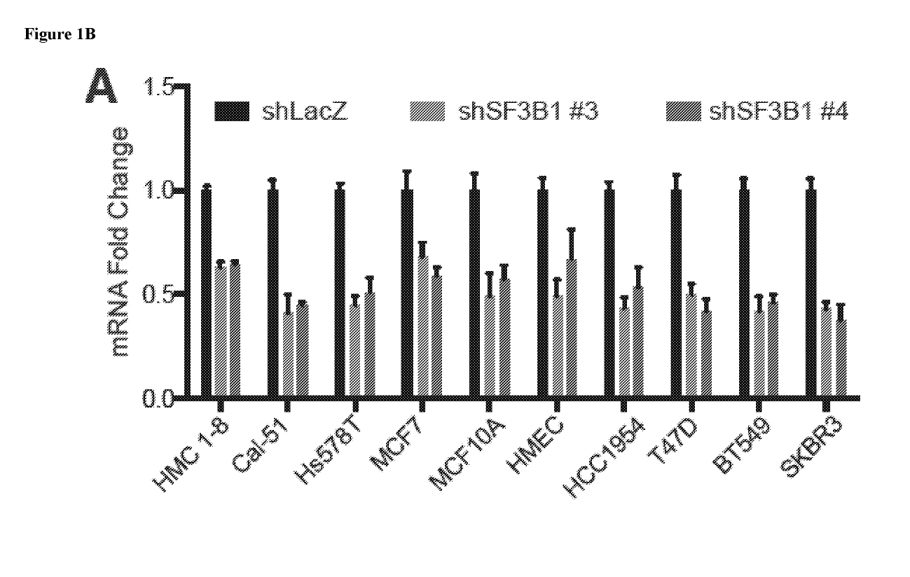

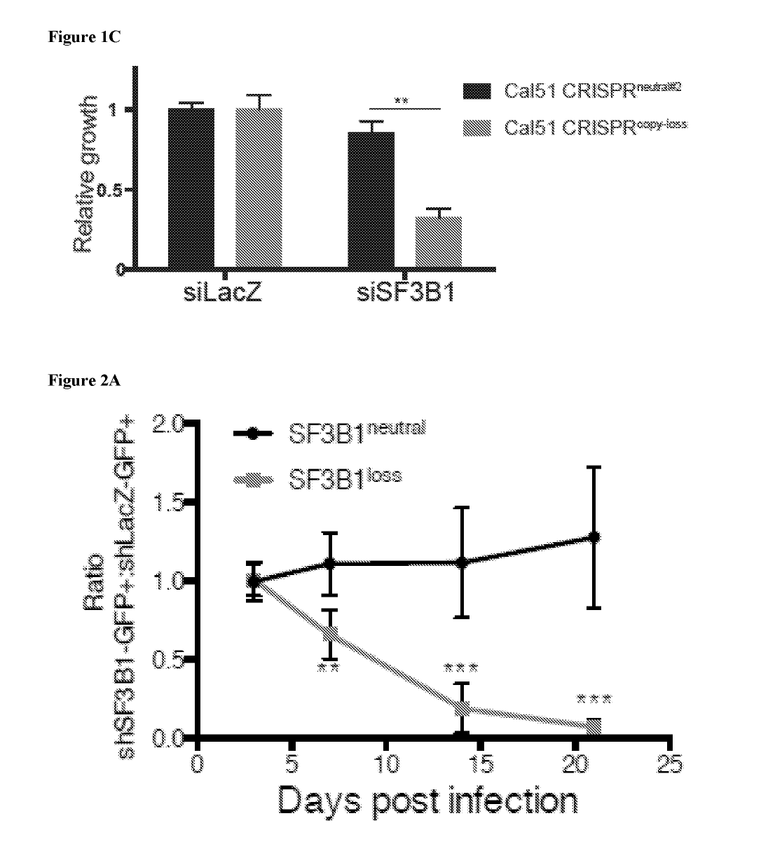

Copy-number alterations Yielding Cancer Liabilities Owing to Partial losS (CYCLOPS) genes were identified using a bioinformatics approach. These genes underwent partial copy loss in cancer, and cells with copy loss of a CYCLOPS gene were more sensitive to suppression of the gene than cells without copy loss. SF3B1 was among the most significant candidate genes in our CYCLOPS analysis. Cells without SF3B1 copy loss (SF3B1^{neutral} cells) including Cal 51, HMC 1-8 and Hs578T, and cells with SF3B1 copy loss (SF3B1^{loss} cells) including HCC1954, BT549 and T47D were obtained. BT549 and T47D have a ploidy of 3.2, while the average copy number of SF3B1 in these cells is about 2. With similar levels of SF3B1 knockdown by two shRNAs targeting SF3B1 (FIG. 1B), SF3B1^{loss} cells underwent significantly slower growth than SF3B1^{neutral} cells (FIG. 1A).

Similar results were generated in isogenic SF3B1^{loss} cells derived from the SF3B1^{neutral} cell line Cal 51. SF3B1^{frameshift-loss} cell were generated using a CRISPR method causing a frameshift mutation inactivating one SF3B1 allele (CRISPR^{frameshift-loss}). The Cal 51 CRISPR^{frameshift-loss}cells were significantly more sensitive to SF3B1 suppression than the cells that were generated in parallel but did not produce inactivating alleles (CRISPR^{neutral} cells) (FIG. 1A). In addition, a second Cal 51 cell line with deletion of one copy of the SF3B1 locus (CRISPR^{copy-loss}) generated by CRISPR using two sgRNAs—one upstream targeting a heterozygous SNP, one downstream of SF3B1—showed significantly slower growth upon SF3B1 suppression compared to Cal 51 CRISPR^{neutral#2} cells generated in parallel without SF3B1 copy loss (FIG. 1C).

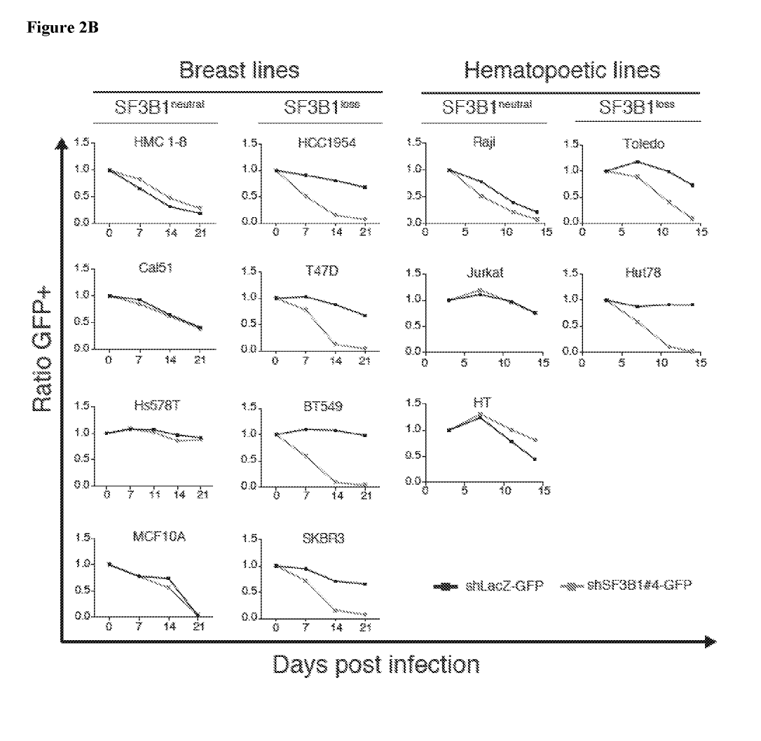

The vulnerability of the SF3B1^{loss} cells to SF3B1 suppression was confirmed using a GFP-competition assay in which the proliferation rate of uninfected cells co-cultured with cells infected with a vector that co-expressed GFP and an shRNA targeting either LacZ or SF3B1 was compared. The expression of LacZ or SF3B1 shRNAs did not result in significant changes in proliferation of SF3B1^{neutral} cells in seven cell lines. In contrast, SF3B1^{loss} cells expressing SF3B1shRNAs did not survive in long-term culture (FIGS. 2A, 2B).

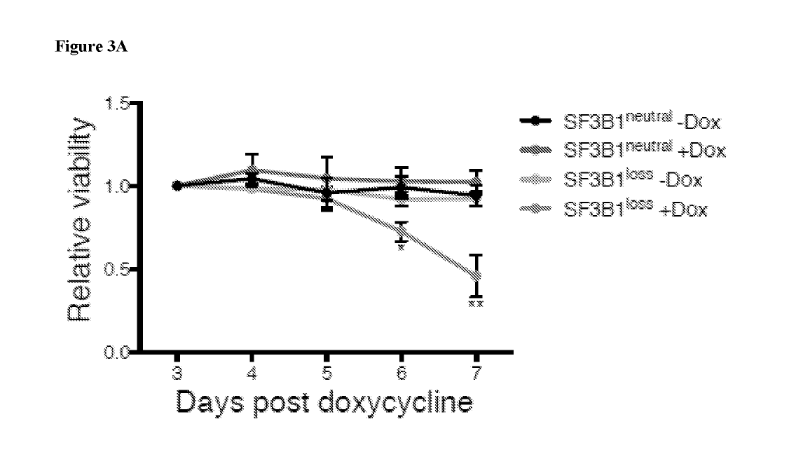

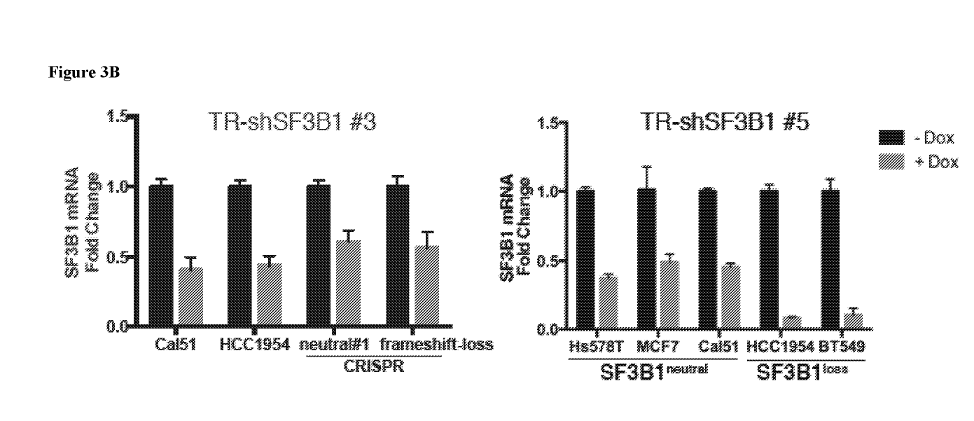

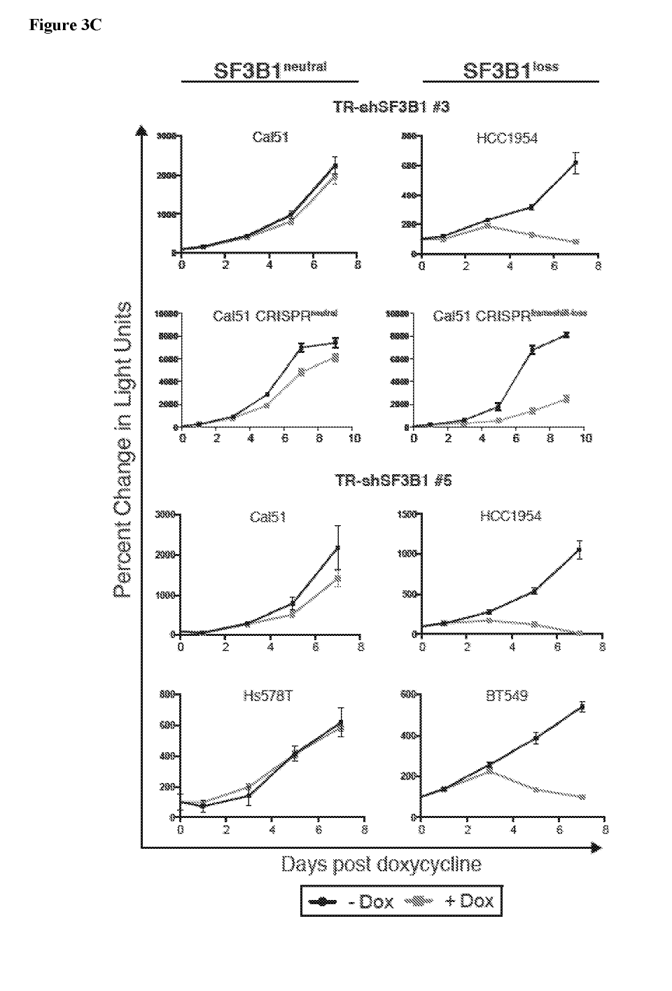

In order to separate the step of infection of virus carrying shRNAs and the step of SF3B1 suppression, cell cultures containing a tetracycline inducible system were generated. SF3B1^{neutral} cells and SF3B1^{loss} cells express hairpins targeting Luciferase or SF3B1 upon doxycycline treatment. Consistent with stable SF3B1 knockdown, inducible SF3B1 knockdown inhibits the growth of SF3B1^{loss} cells but not SF3B1^{neutral} cells (FIGS. 3A, 3B, 3C).

### Example 3

### SF3B1 Suppression Led to Cell Cycle Arrest and Cell Death of Cells with SF3B1 Copy Loss

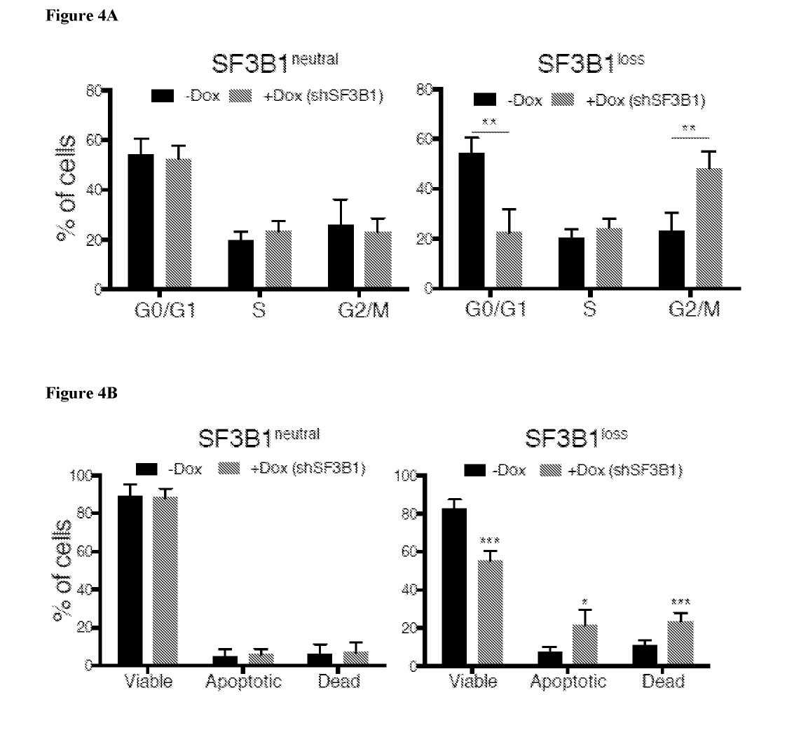

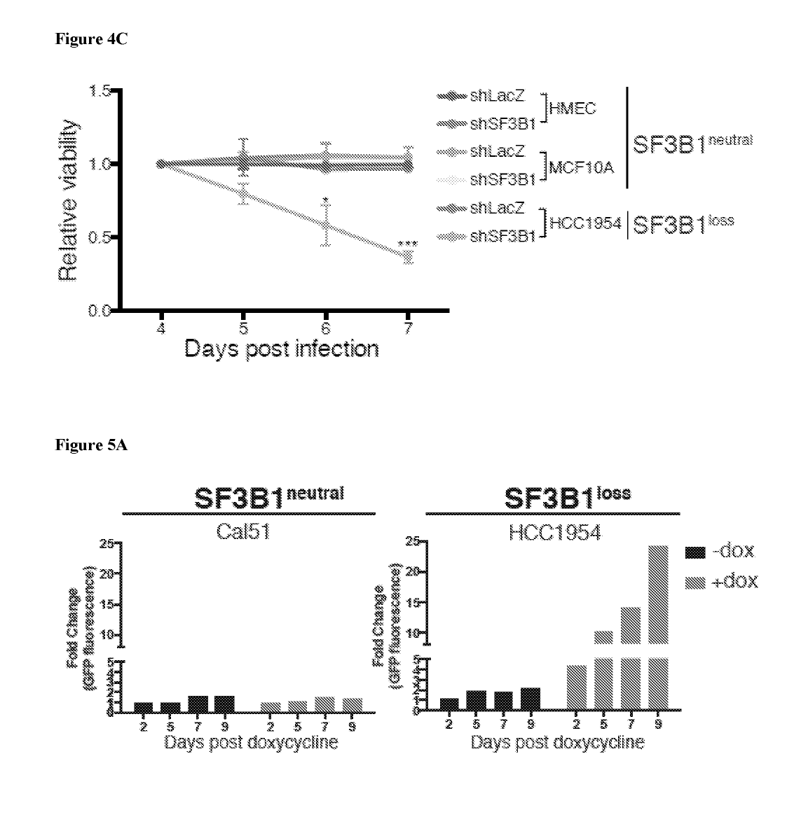

From a cell cycle analysis, it was found that SF3B1^{loss} cells had significantly increased proportions of cells in G2/M phase after SF3B1 knockdown, which did not occur in SF3B1^{neutral} cells (FIG. 4A). In addition to the aberrant cell cycle progression, SF3B1^{loss} cells exhibited a significant induction in cell death following SF3B1 knockdown. They underwent apoptosis as determined by increased number of AnnexinV-positive/propidium iodide (PI)-positive cells and AnnexinV-positive/PI-negative cells, which did not occur in SF3B1^{neutral} cells (FIG. 4B). Similarly, the fraction of viable cells, as determined by the exclusion of propidium iodide, significantly decreases only in SF3B1^{loss} cells expressing SF3B1 shRNA (“shSF3B1”), not in SF3B1^{neutral} cells expressing SF3B1 shRNA (“shSF3B1”) or SF3B1^{loss} cells expressing LacZ shRNA (“shLacZ”) (FIG. 4C).

### Example 4

### Complementation of SF3B1 Expression Rescued the Growth of SF3B1^{loss} Cells with SF3B1 Suppression

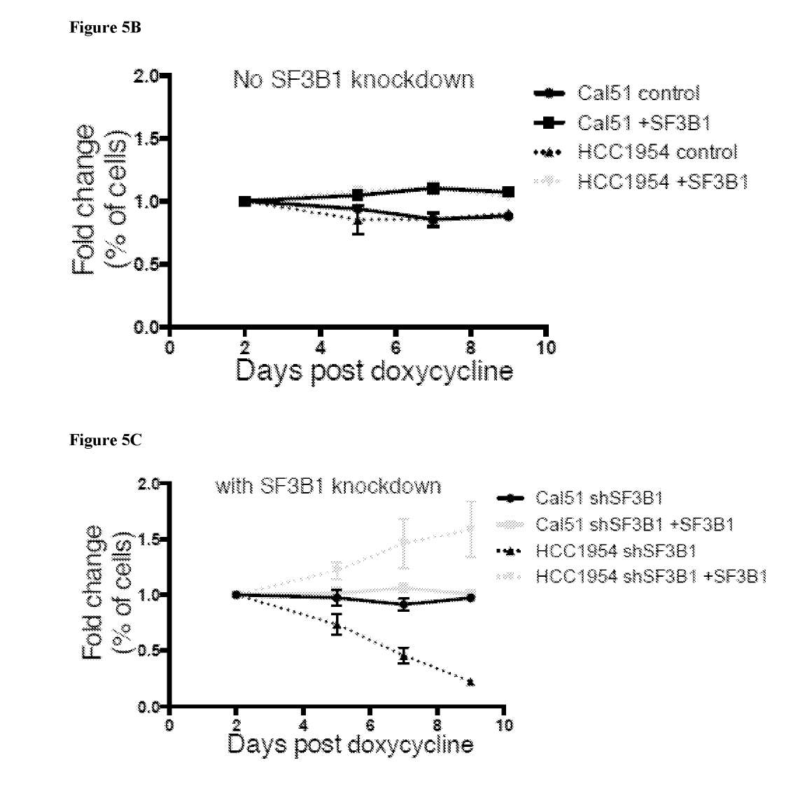

A lentiviral construct was used to confirm the specificity of the SF3B1-targeting shRNAs. The construct expressed a codon-optimized SF3B1 ORF, which is resistant to shRNA suppression, fused to an IRES GFP sequence (SF3B1WT-IRES-GFP). The expression level of SF3B1WT-IRES-GFP did not change during Dox induction of SF3B1 shRNA in SF3B1^{neutral} cells, but increased by over 20 fold in SF3B1^{loss} cells upon Dox-induced expression of SF3B1 shRNA (FIG. 5A). Therefore, SF3B1-IRES-GFP was more highly expressed in SF3B1^{loss} cells after SF3B1 knockdown than in SF3B1^{neutral} cells. When placed in competition, cells infected or not infected with SF3B1WT-IRES-GFP maintained constant ratios over 10 days (FIG. 5B), suggesting that short-term expression of SF3B1 does not alter cellular fitness in either SF3B1^{neutral} or SF3B1^{loss} cells. Next, endogenous SF3B1 was knocked down in all cells and expressed SF3B1WT-IRES-GFP in 50% of cells. While SF3B1^{neutral} cells were not affected by SF3B1 suppression, SF3B1^{loss} cells expressing an SF3B1 shRNA failed to survive in long-term culture. Remarkably, SF3B1^{loss} cells expressing both an SF3B1 shRNA and SF3B1WT-IRES-GFP persisted in long-term culture (FIG. 5C), indicating that complementary expression of SF3B1 was sufficient to prevent cell death.

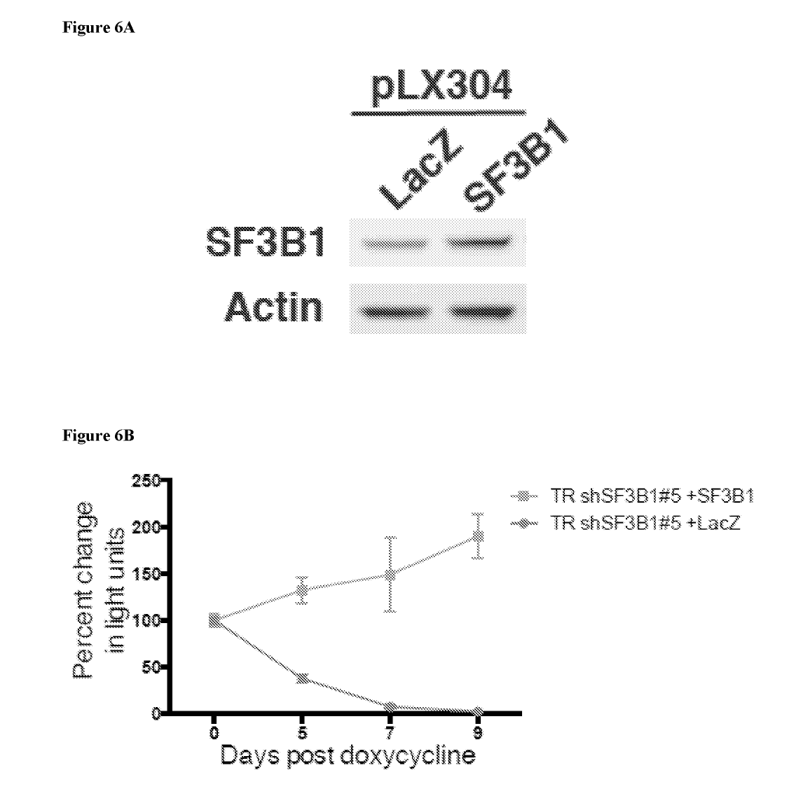

Cell lines with stable exogenous expression of LacZ or SF3B1 were also established. The expression of SF3B1 was sufficient to restore the proliferation of SF3B1^{loss} cells expressing an SF3B1-targeting shRNA (FIGS. 6A, 6B).

### Example 5

### SF3B1^{neutral} Cells Contained Excess SF3B1 Beyond the Requirement for Survival

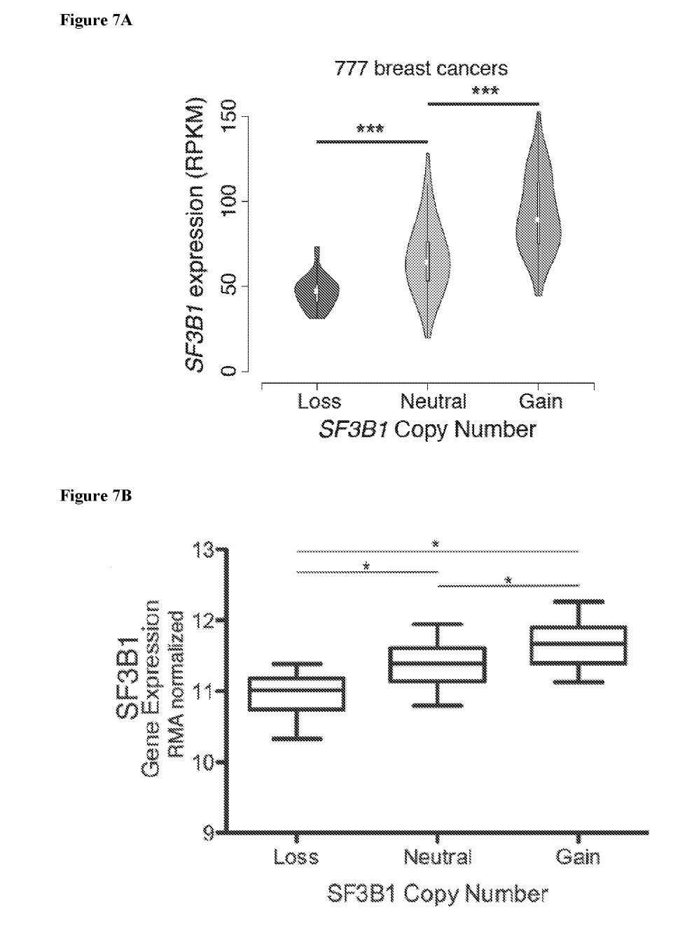

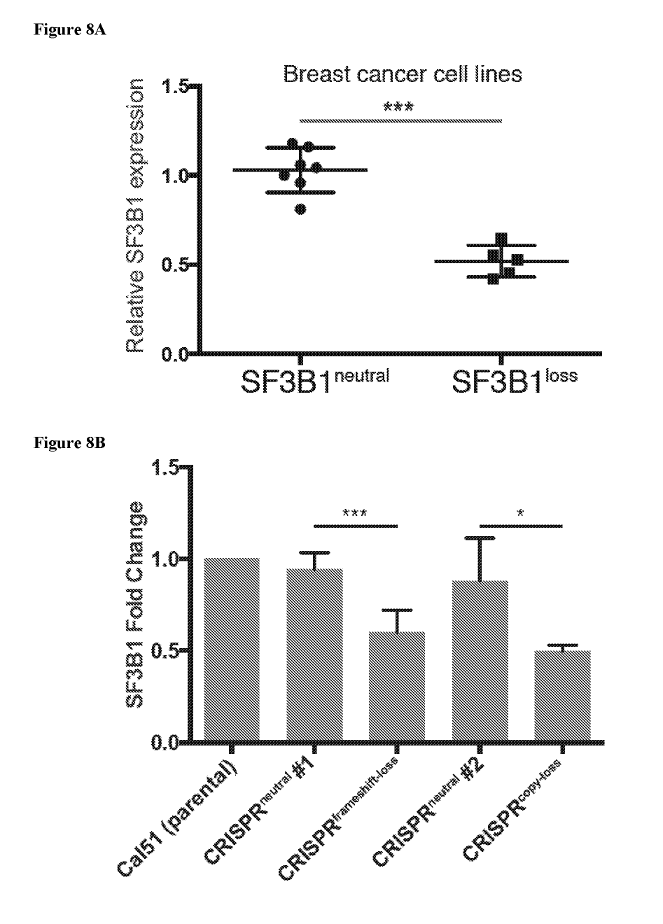

Analyses of SF3B1 mRNA indicate that SF3B1neutral cells tolerate partial SF3B1 suppression because they express more SF3B1 than they require. In both TCGA breast adenocarcinoma data (777 samples) (Network, 2012) and the Cancer Cell Line Encyclopedia (CCLE; 947 cell lines), SF3B1^{neutral} samples had significantly higher expression of SF3B1 mRNA relative to SF3B1^{loss} samples (FIGS. 7A, 7B; TCGA Mann-Whitney p<1×10⁻⁴, CCLE Mann-Whitney p<1×10⁻⁴), suggesting excess mRNA over requirements for survival. It was validated that SF3B1neutral breast cancer cell lines (n=7) express approximately twice as much SF3B1 mRNA as SF3B1loss cells (n=5) by quantitative PCR (FIG. 8A; p<1×10⁻⁴) and found similar SF3B1 expression changes between the CRISPR^{neutral} and CRISPR^{loss} lines; FIG. 8B).

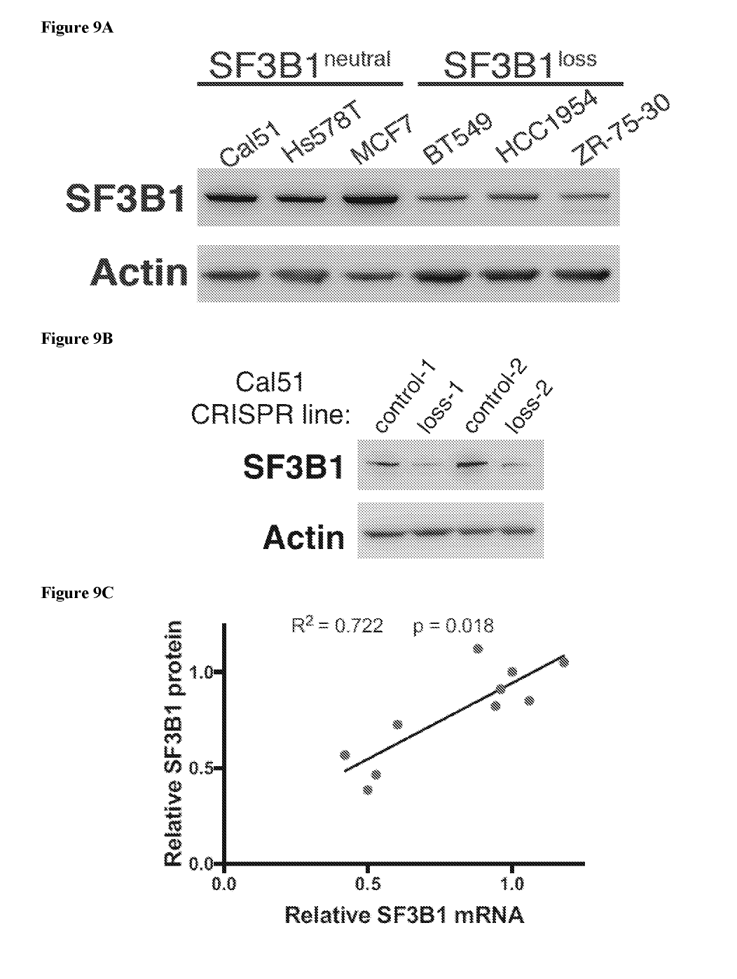

These differences in SF3B1 mRNA expression were recapitulated at the protein level. Among breast cancer lines, Western blots indicated increased SF3B1 protein expression in SF3B1^{neutral} compared to SF3B1^{loss} cells (FIG. 9A) and these differences were recapitulated in CRISPR^{neutral} vs. CRISPR^{loss} cells (FIG. 9B). A significant linear correlation between SF3B1 mRNA and protein expression was also found in a panel of breast cancer cell lines (FIG. 9C, p=0.0018, R2=0.772).

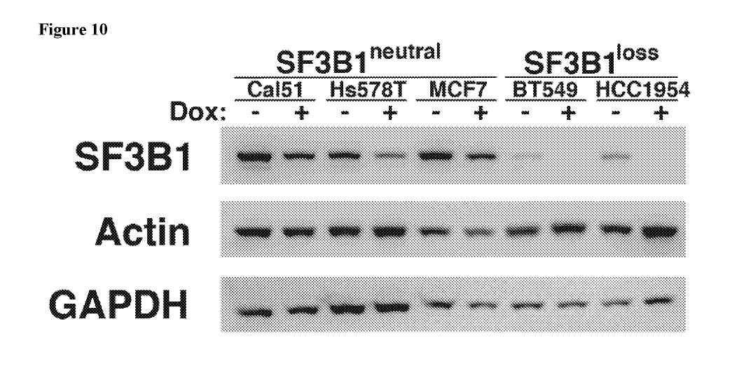

These observations suggest that SF3B1^{neutral} cells tolerate partial SF3B1 suppression because moderate SF3B1 suppression leaves them with sufficient residual protein for survival. Indeed, immunoblots of SF3B1^{neutral} cells after SF3B1 suppression indicated detectable SF3B1 levels, whereas no protein could be detected in SF3B1^{loss} cells after SF3B1 suppression (FIG. 10).

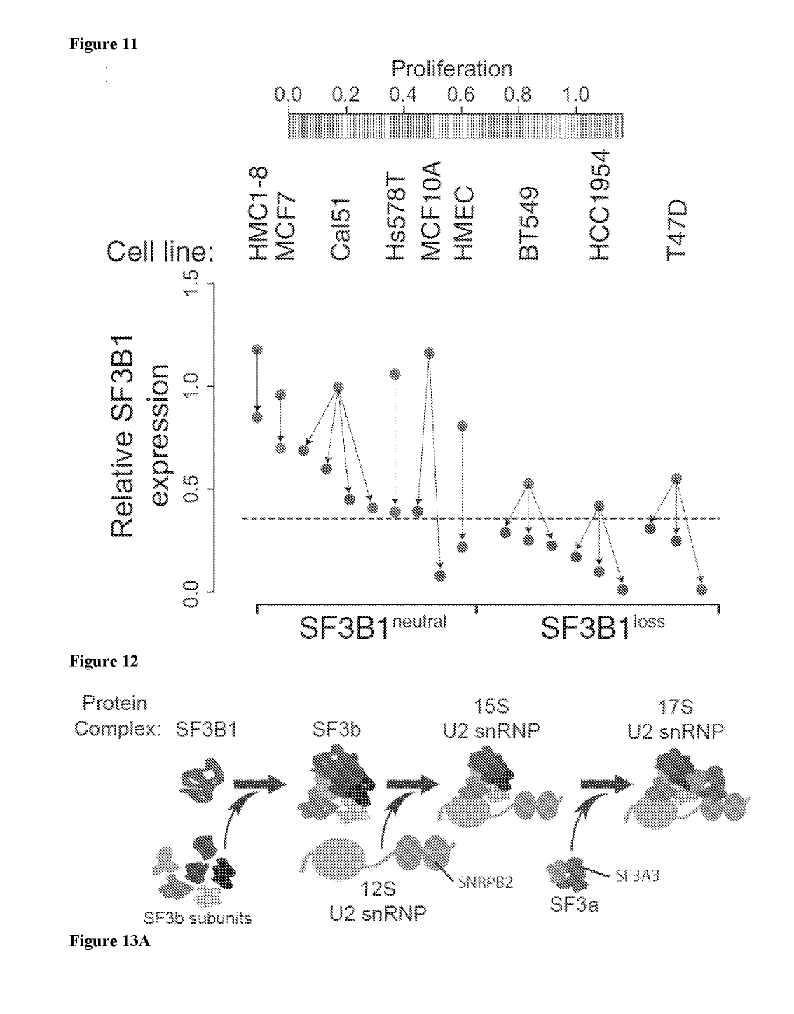

A systematic analysis of shRNA-induced mRNA suppression across SF3B1^{neutral} and SF3B1^{loss} lines indicated that SF3B1 mRNA levels can be reduced by 60% from SF3B1^{neutral} cell basal levels before proliferation defects are apparent. As shown in FIG. 11, loss of half of the copies of SF3B1 does not significantly compromised cell survival, but further reduction of SF3B1 expression by 10% or more, or reduction of SF3B1 expression from a cell having SF3B1 copy loss by 20% or more, substantially inhibited cell proliferation. SF3B1 expression was suppressed using shRNAs with different potency to generate a range of SF3B1 suppression in SF3B1^{neutral} and SF3B1^{loss} cells. Although similar reductions in SF3B1 expression were obtained in SF3B1^{neutral} and SF3B1^{loss} lines, the elevated basal levels of SF3B1 expression in SF3B1^{neutral} lines enabled them to retain sufficient SF3B1 for proliferation despite shRNA expression.

### Example 6

### SF3B1 Copy-Loss Selectively Reduced the Abundance of the SF3b Complex

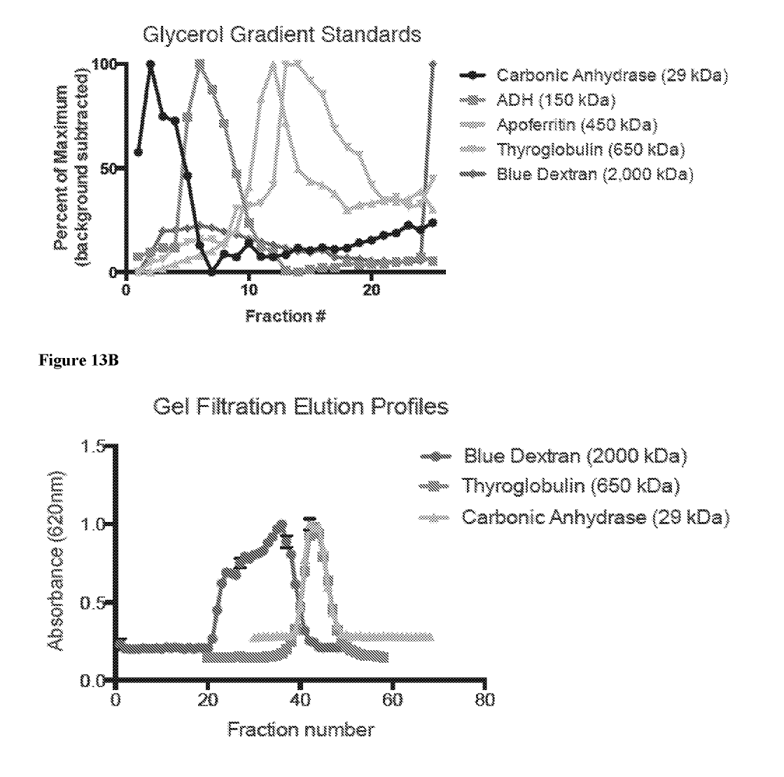

SF3B1 is a component of the seven-member SF3b sub-complex of the U2 snRNP. Incorporation of SF3b into the U2 snRNP_12S “core” forms the 15S U2 snRNP, which combines with SF3a to form the mature 17S U2 snRNP (FIG. 12). The expression levels of native SF3B1-containing complexes from whole-cell extracts were assessed by glycerol gradient sedimentation and gel filtration chromatography. Protein complexes from 29-650 kDa and 650-2,000 kDa were resolved using 10-30% glycerol gradients and Sephacryl S-500 gel filtration chromatography, respectively (FIGS. 13A, 13B). This enabled resolution of SF3B1-containing complexes ranging from monomers (155 kDa) to the SF3b sub-complex (450 kDa) to the 15S and 17S U2 snRNPs (790 and 987 kDa, respectively).

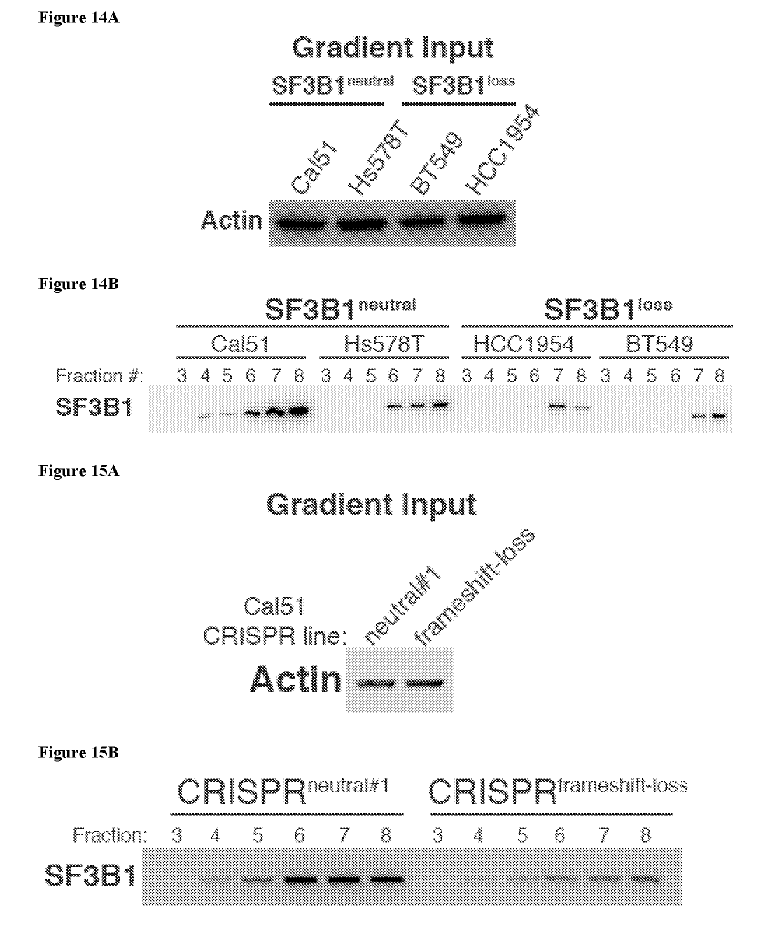

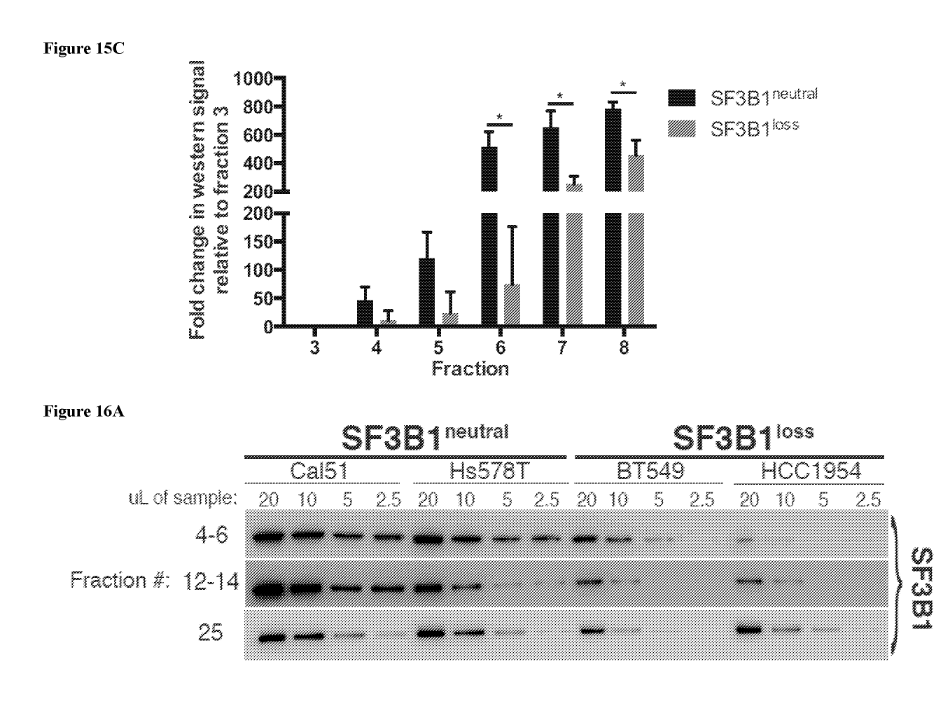

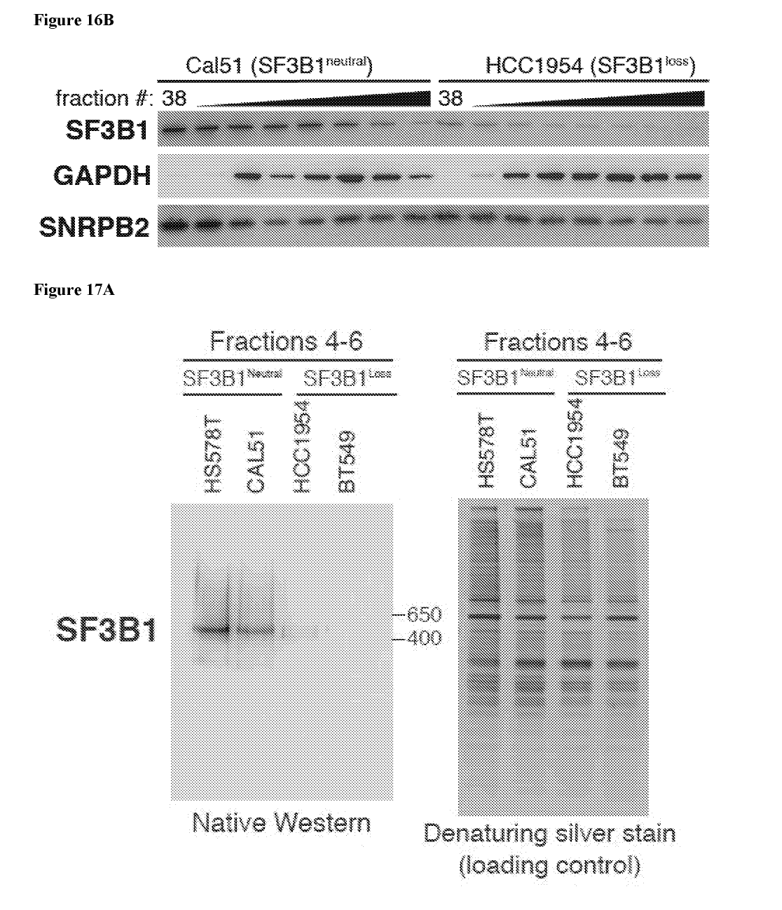

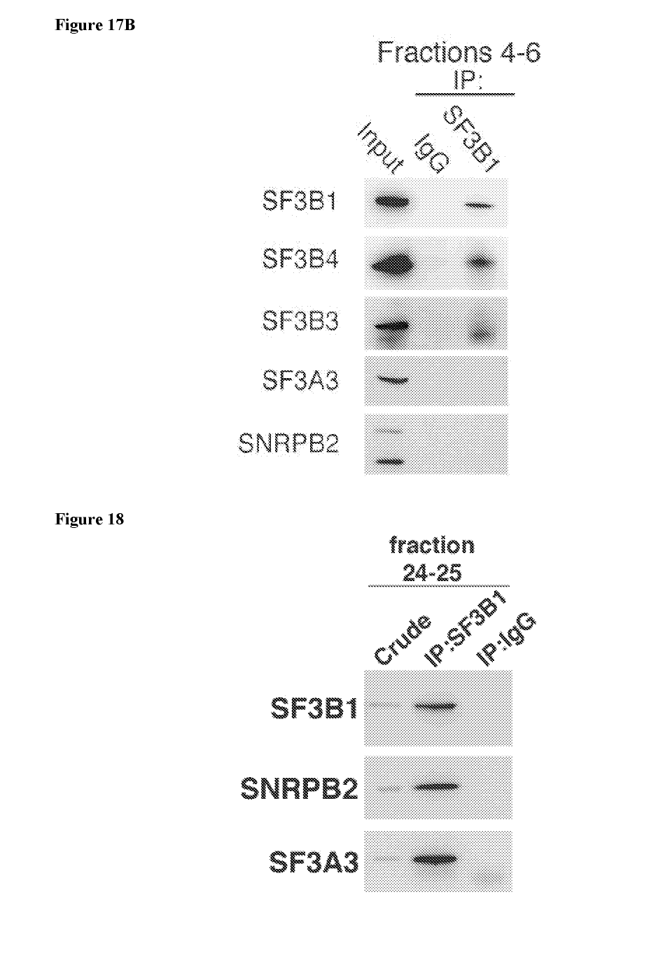

The elution profiles between patient-derived and isogenic SF3B1^{loss} and SF3B1^{neutral} cells were compared. Substantially lower levels of SF3b were observed in the SF3B1^{loss} cells. The largest decreases in SF3B1-containing complexes in glycerol gradients were in fractions 4-6, corresponding to ˜29-450 kDa (FIGS. 14A, 14B, 15A, 15B), and fractions 12-14, corresponding to ˜450-650 kDa (FIG. 16A). Similar decreases in gel filtration chromatography fractions corresponding to complexes <650 kDa were observed (FIG. 16B). Native western blotting from the pooled glycerol gradient fractions 4-6 indicated the loss of a single SF3B1-containing complex of 450 kDa (FIG. 17A). SF3B1 immunoprecipitation from these fractions resulted in the coprecipitation of SF3b components SF3B3 and SF3B4 in SF3B1^{neutral} cells, but not of U2 snRNP components SNRPB2 and SF3A3 (FIG. 17B).

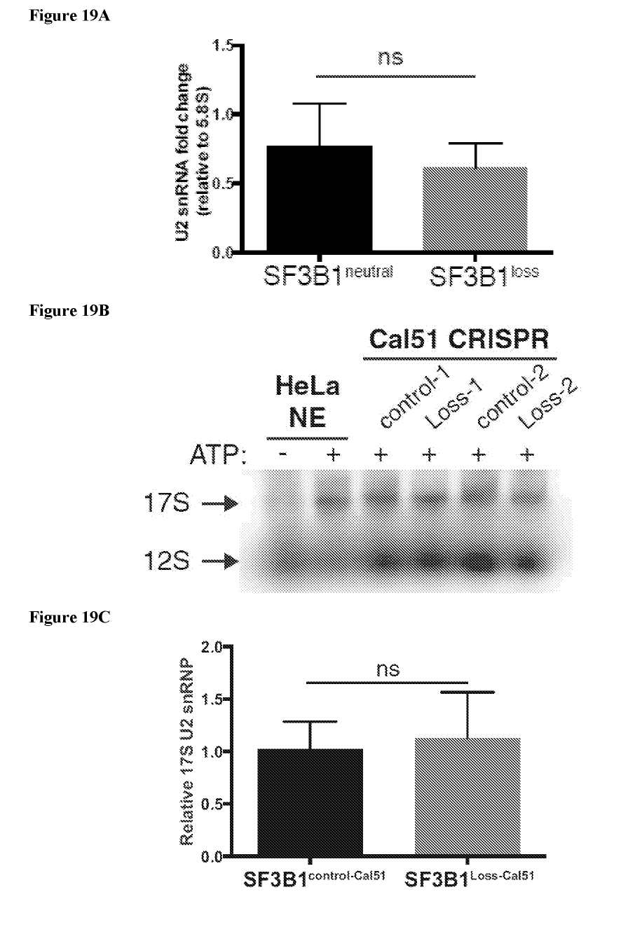

Conversely, U2 snRNP levels were only modestly decreased in SF3B1^{loss} lines. Levels of SF3B1 in glycerol gradient fraction 25 (corresponding to >650 kd and containing the U2 snRNP) were only slightly decreased in SF3B1¹⁰’ relative to SF3B1^{neutral} lines (FIG. 16A). SF3B1 immunoprecipitation from fractions 24-25 resulted in co-precipitation of U2 snRNP components SNRPB2 and SF3A3 (FIG. 18). U2 snRNA levels are known to track with U2 snRNP levels, and no significant difference in U2 snRNA abundance between SF3B1^{neutral} and SF3B1^{loss} lines was observed, although there was a trend towards lower expression in the SF3B1^{loss} lines (FIG. 19A; p=0.35, two-tailed t-test). Similarly, visualization of U2 snRNP complexes using radiolabeled oligonucleotides complementary to the U2 snRNA did not demonstrate differences in 17S U2 snRNP abundance in SF3B1loss cells (FIGS. 19B and 19C).

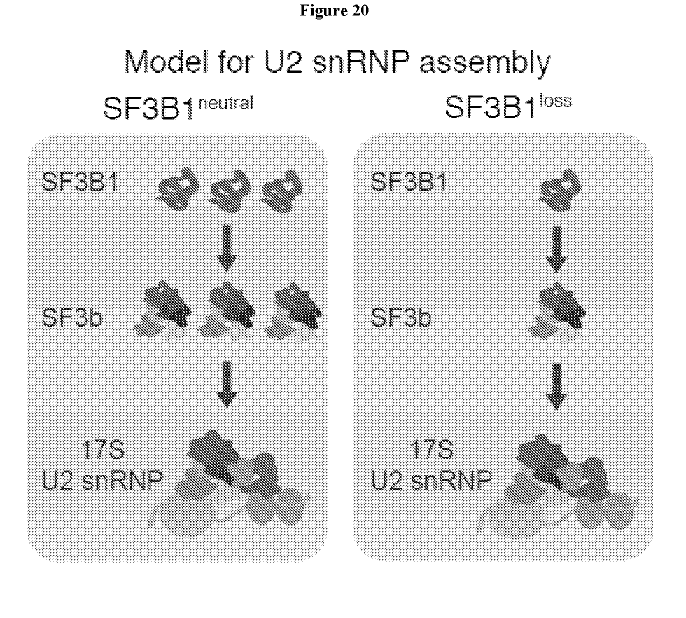

These data suggested that copy-loss of SF3B1 only modestly affected U2 snRNP abundance but substantially decreased levels of U2 snRNP precursor complexes under steady-state conditions (FIG. 20).

### Example 7

### SF3B1 Suppression Selectively Reduced U2 snRNP Abundance in SF3B1^{loss} Cells

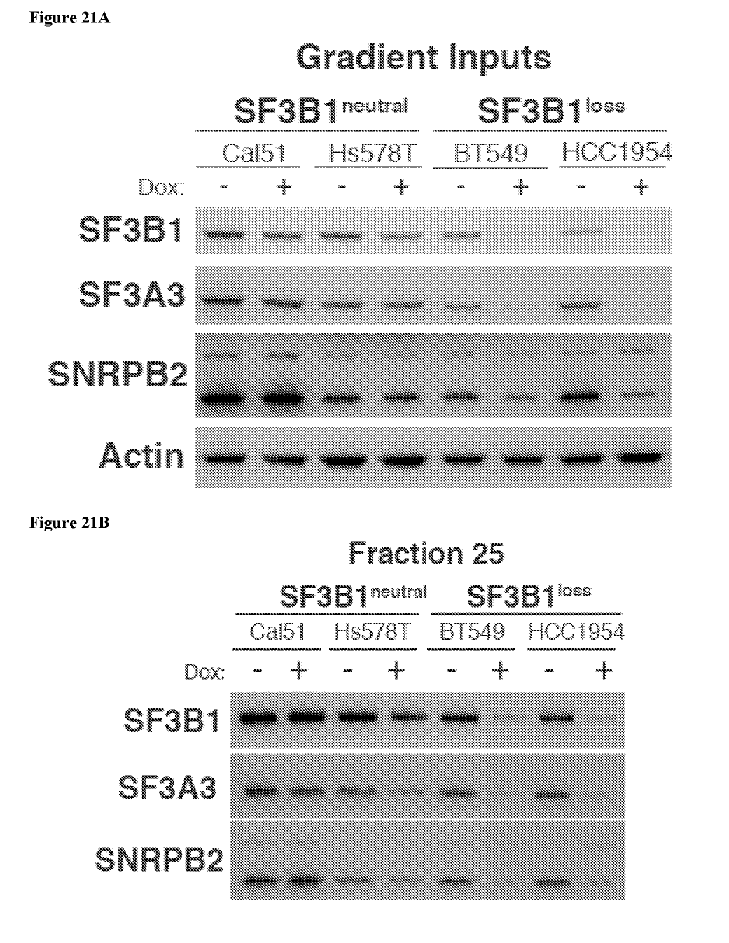

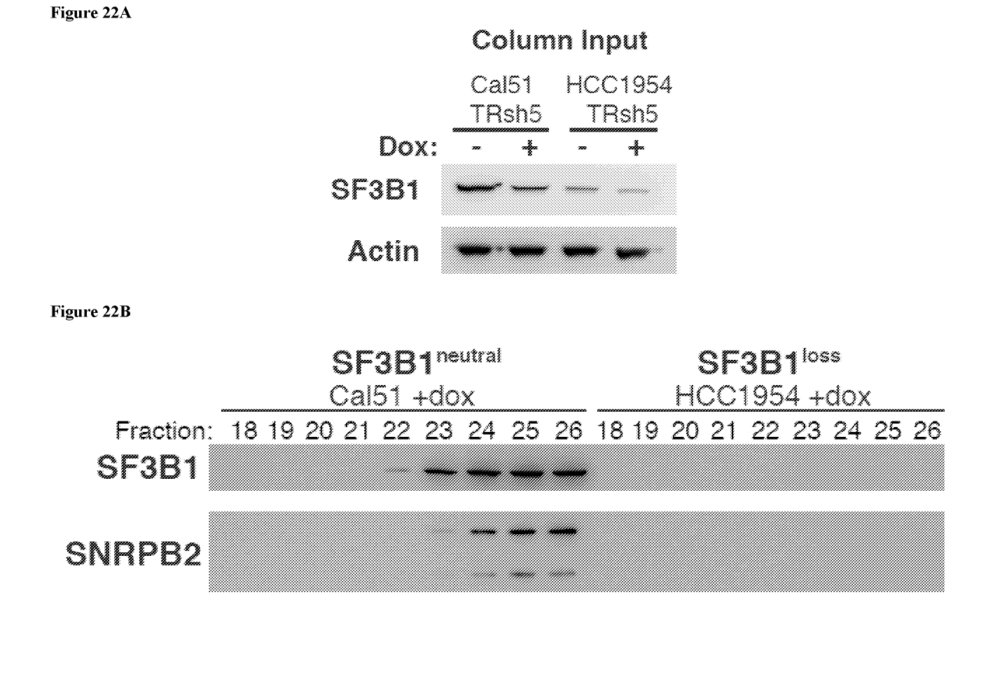

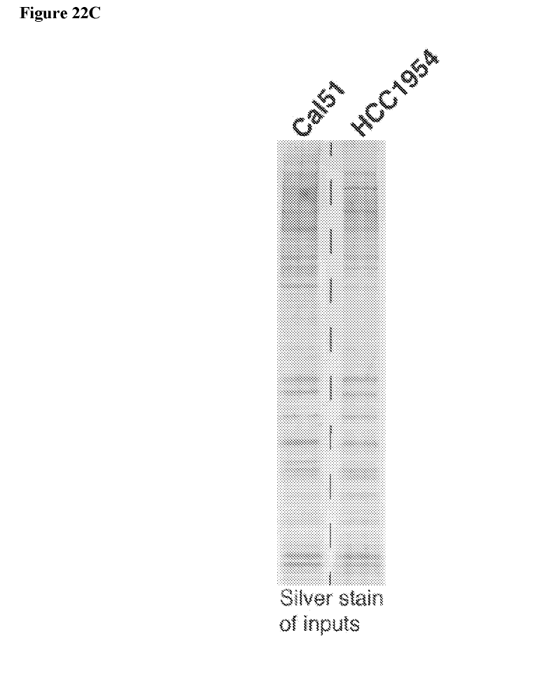

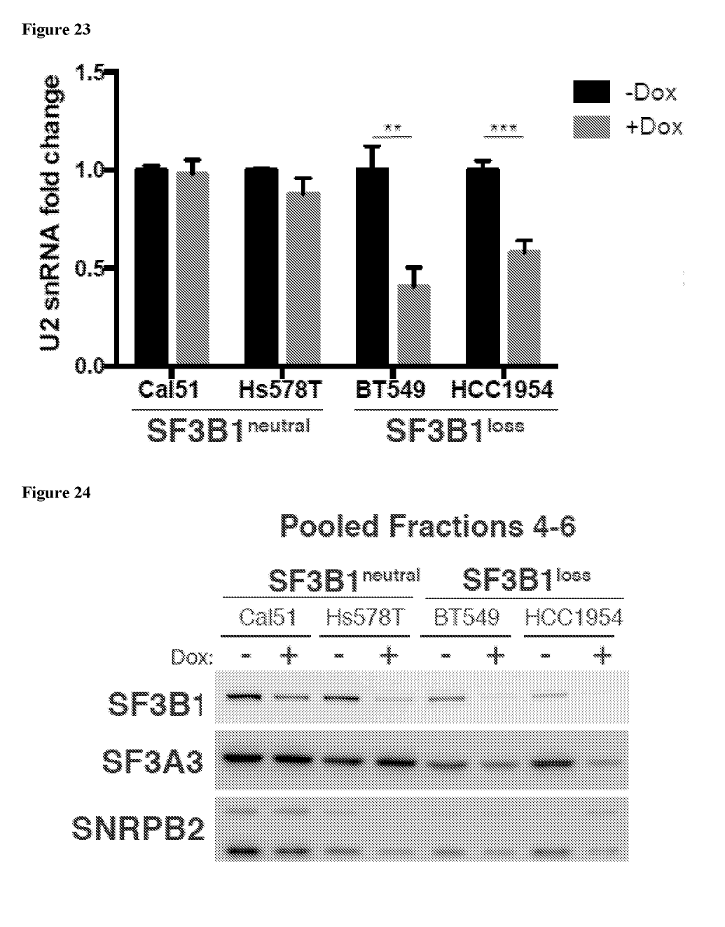

Suppression of SF3B1 led to substantial reductions of U2 snRNP levels in SF3B1^{loss} but not SF3B1^{neutral} cells. Although such suppression resulted in reduced SF3B1 levels in both SF3B1loss and SF3B1neutral lines, only the SF3B1^{loss} lines exhibited concomitant reductions in levels of U2 snRNP components SF3A3 and SNRPB2 (FIG. 21A). These decreases were observed in glycerol gradient fraction 25, corresponding to the U2 snRNP, only in SF3B1^{loss} lines (FIG. 21B). Furthermore, after SF3B1 suppression, both SF3B1 and SNRPB2 were detected in Sephacryl-S500 fractions containing >650 kd protein complexes in SF3B1^{neutral} cells but not in SF3B1^{loss} cells (FIGS. 22A, 22B, 22C). Quantitative PCR also indicated significantly reduced U2 snRNA expression after SF3B1 suppression in SF3B1^{loss} cells but not in SF3B1^{neutral} cells (FIG. 23).

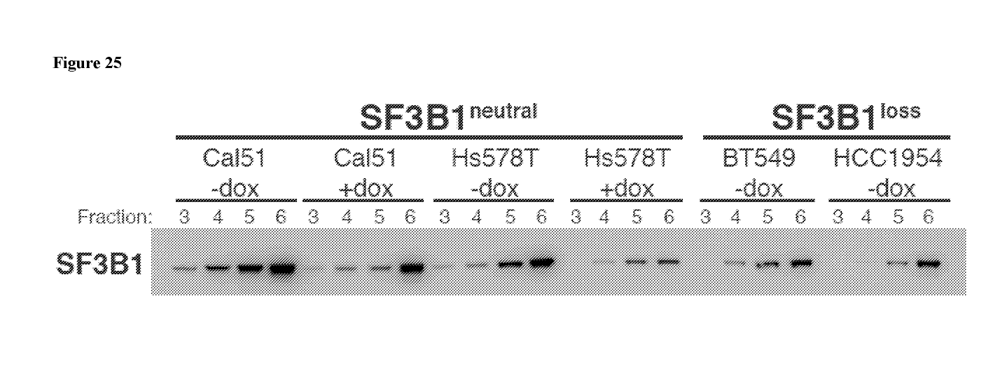

Conversely, suppression of SF3B1 in SF3B 1^{neutral} cells decreased levels of SF3b, but not the U2 snRNP. SF3B1 suppression did not reduce SF3B1 in fraction 25 (FIG. 21B) but instead preferentially reduced SF3B1 from fractions 4-6 (FIG. 24) in SF3B1^{neutral} cells. Further, no changes in SF3A3 or SNRPB2 expression were observed in total protein from glycerol gradient inputs (FIG. 21A) or U2 snRNA expression (FIG. 23). SF3B1^{neutral} cells with SF3B1 suppression reduced SF3b levels in glycerol gradient fractions 3-6 approximately to the levels observed in SF3B1^{loss} cells (FIG. 25), thereby phenocopying the reduced SF3b observed in unperturbed SF3B1^{loss} cells. Taken together, these data suggested that the elevated levels of the SF3b sub-complex in SF3B1^{neutral} cells relative to SF3B1^{loss} cells buffered SF3B1^{neutral} cells from reductions in viability following SF3B1 suppression.

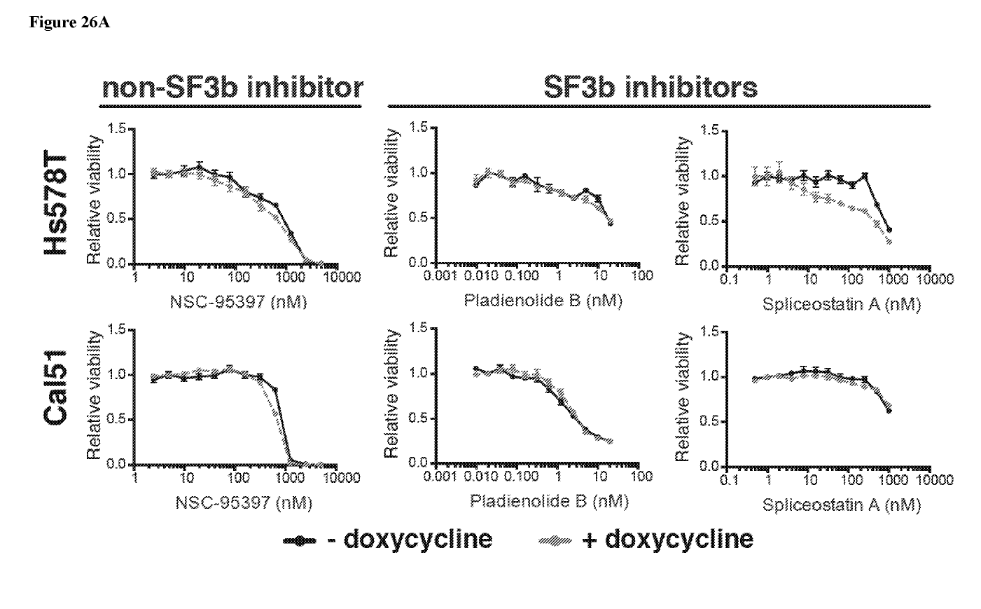

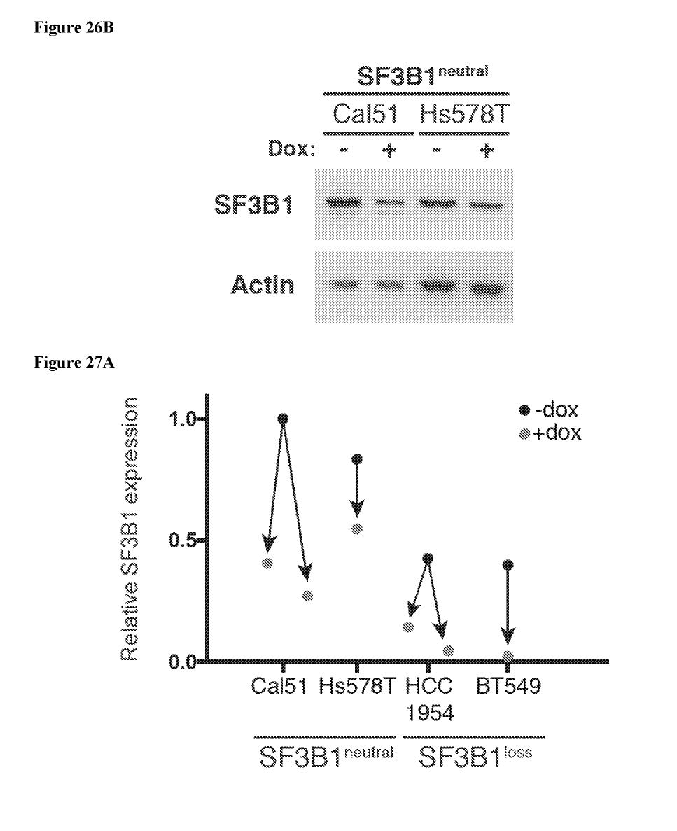

The reduction of U2 snRNP levels specifically in SF3B1^{loss} cells by SF3B1 suppression (FIGS. 16A, 19, 21B) suggested that existing SF3b inhibitors, which prevent U2 snRNP function or subsequent steps during splicing catalysis, might exploit the specific vulnerability exhibited by SF3B1^{loss} cells. Indeed, Spliceostatin A, an SF3b-targeting compound, led to increased cell death of Hs578T breast cancer cells when suppression of SF3B1 expression is induced, whereas NSC95397, a compound reported to inhibit splicing activity by an SF3b-independent mechanism, failed to exhibit increased effects on cells with SF3B1 copy-loss or suppression (FIGS. 26A, 26B).

In addition to SF3B1 RNA interference and SF3B inhibition, SF3B1 suppression can be exerted by deubiquitinase (DUB) inhibition. Treatment of Cal51 cells by three different DUB inhibitors PR-619, b-AP15 and SJB3-019A each led to significant decrease of of SF3B1 expression (FIG. 37). Therefore, DUB inhibitors may be capable of killing SF3B1^{loss} cells specifically.

### Example 8

### SF3B1 Suppression Resulted in Splicing Defects in SF3B1loss Cells

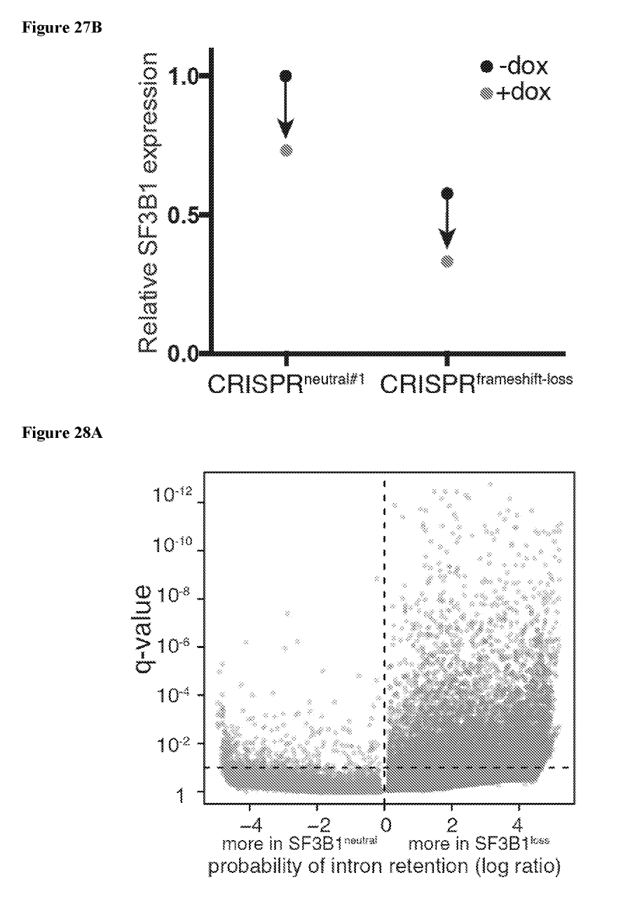

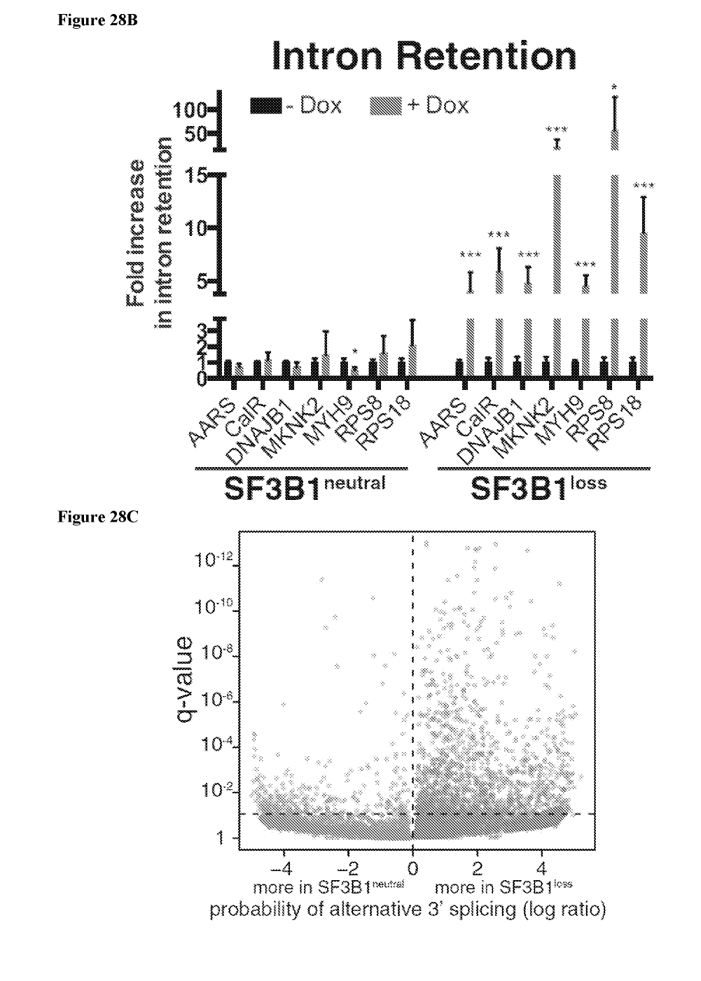

SF3B1 is well-established as a splicing factor, as demonstrated by intron retention upon treatment of cells with SF3B1 inhibitors and in patients harboring SF3B1 mutations. RNA sequencing was performed to quantify the extent of splicing disruption upon SF3B1 suppression in SF3B1^{neutral} and SF3B1^{loss} cells (FIGS. 27A, 27B), and juncBase and a novel statistical framework were used to analyze 50,600 splice junctions for intron retention from the RNA sequencing data. All cells showed evidence of increased intron retention following SF3B1 suppression (p<10⁻⁵), but splicing was significantly more affected in SF3B1^{loss} cells. Upon SF3B1 suppression, 7038 transcripts in SF3B1^{loss} cells showed evidence of significantly (q<0.01) increased intron retention relative to SF3B1^{neutral} cells, whereas only 298 transcripts showed evidence of increased intron retention in the reverse direction (FIG. 28, p<10⁻¹⁶⁶⁷).

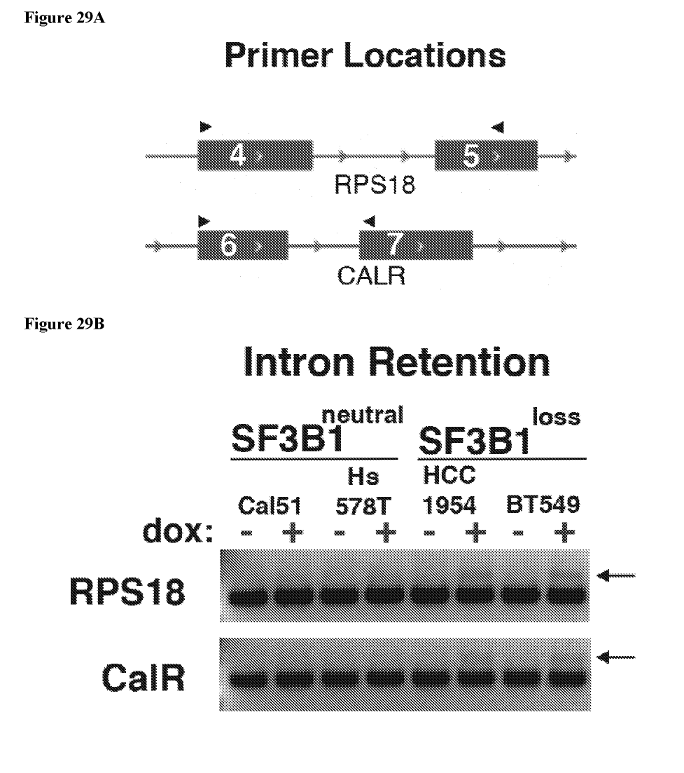

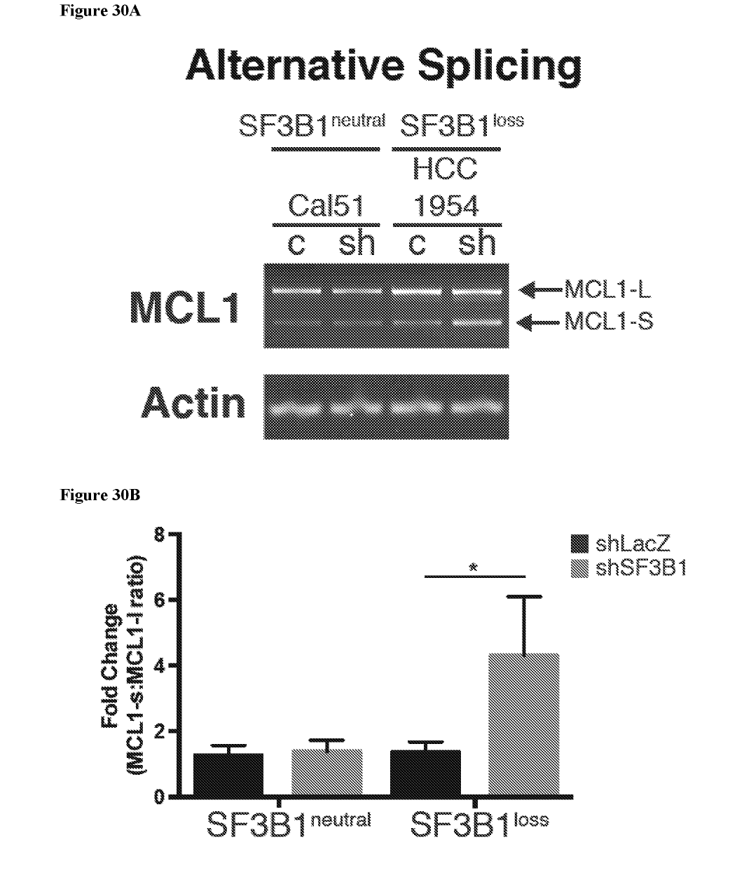

Alterations in splicing was confirmed by RT-PCR of two ubiquitously expressed genes (RPS18 and CALR) that flank short introns amenable to PCR detection if they are improperly retained (FIG. 29A). Upon SF3B1 knockdown, SF3B1^{loss} cells contained transcripts with retained introns that were not observed in SF3B1^{neutral} cells (FIG. 29B). Alterations in alternative splicing was also observed. Specifically, the ratio between alternative long and short isoforms of MCL1 (that respectively do or do not have anti-apoptotic functions) is known to be regulated by SF3B1. After SF3B1 suppression, this ratio was significantly biased towards the short isoform in SF3B1^{loss} cells relative to SF3B1^{neutral} cells (FIGS. 30A, 30B).

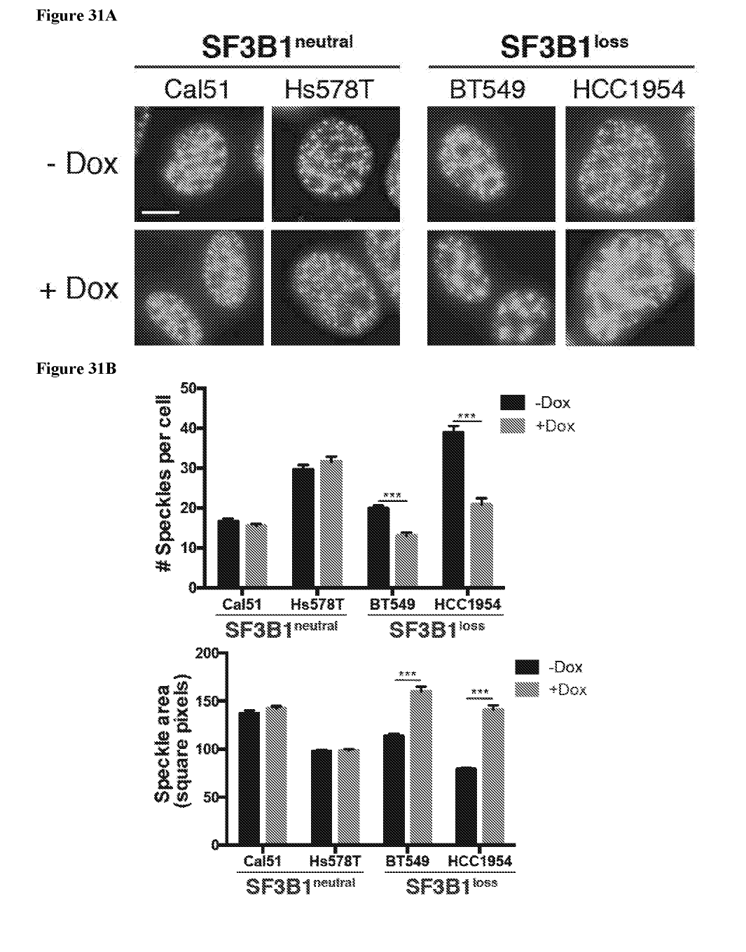

Spliceosome components, including SF3B1, are thought to assemble and function in sub-nuclear compartments known as nuclear speckles. Inhibition of splicing or transcription has been shown to induce formation of enlarged ‘mega-speckles’. An unbiased quantification of the number and size of nuclear speckles per nucleus, represented by immunostaining of nuclear speckle marker SC-35, was performed using a custom image analysis pipeline with CellProfiler software. SF3B1^{neutral} cells did not display changes in SC-35⁺ speckles after SF3B1 suppression, but SF3B1^{loss} nuclei contained significantly fewer speckles and increased speckle area (FIGS. 31A, 31B). The presence of defective alternative splicing, intron retention and formation of mega-speckles uniquely in SF3B1^{loss} cells after SF3B1 suppression further supported the gross defects in splicing observed by RNA sequencing.

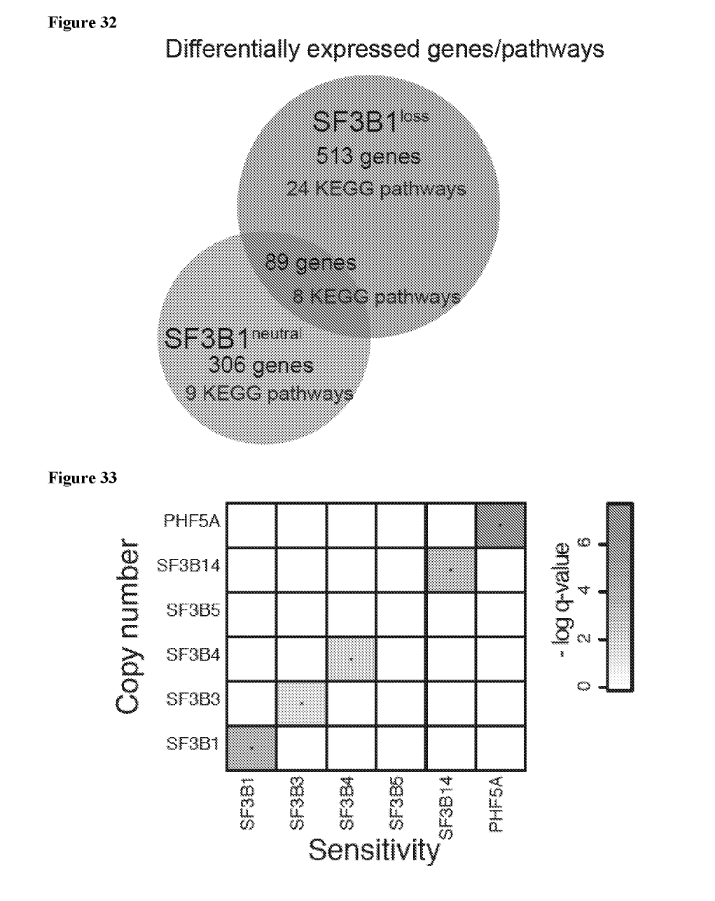

Moreover, upon SF3B1 suppression, 513 genes were differentially expressed at a false discovery rate (FDR) <10% in SF3B1^{loss} cells and only 306 genes were differentially expressed in SF3B1^{neutral} cells (p<0.0001 by Fischer's exact test). Gene set enrichment analysis revealed 24 KEGG pathways significantly enriched in SF3B1^{loss} cells and only 9 pathways altered in SF3B1^{neutral} cells (FIG. 32). These data were consistent with our other data showing that SF3B1 suppression more severely impacts the transcriptome of SF3B1^{loss} cells.

### Example 9

### Suppression of Other SF3B Complex Subunits Did Not Lead to Increased Vulnerability of SF3B1^{loss} Cells

An extended CYCLOPS analysis was performed to interrogate whether suppression of other genes, especially those encoding proteins in the SF3B1 complex, may inhibit growth of SF3B1^{loss} cells more efficiently than SF3B1^{neutral} cells. It was found that copy number alterations of these genes did not confer susceptibility to SF3B1 suppression. The significance of associations between Achilles RNAi sensitivity data of each SF3b complex subunit and copy numbers of each SF3b complex member in SF3B1loss cells was calculated (FIG. 33). Six of seven SF3b subunits were analyzed (SF3B1, SF3B3-5, SF3B14 and PHF5A), and no associations between susceptibility to suppression of any of these genes and copy numbers of other SF3b subunits including SF3B1 was observed.

### Example 10

### Suppression of SF3B1 Reduces Tumor Growth in SF3B1^{loss} Xenografts

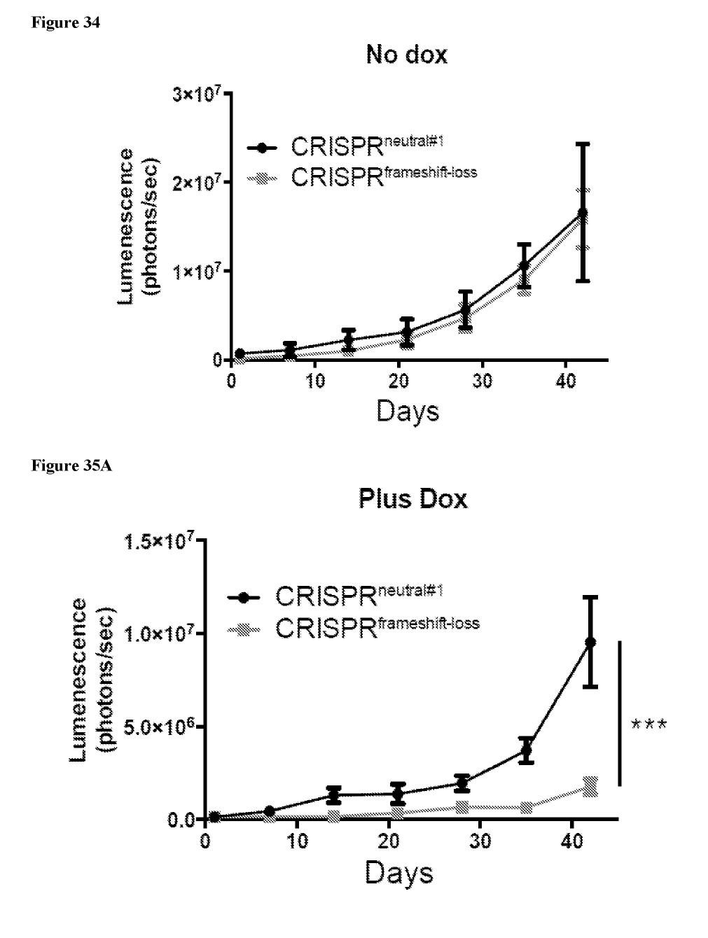

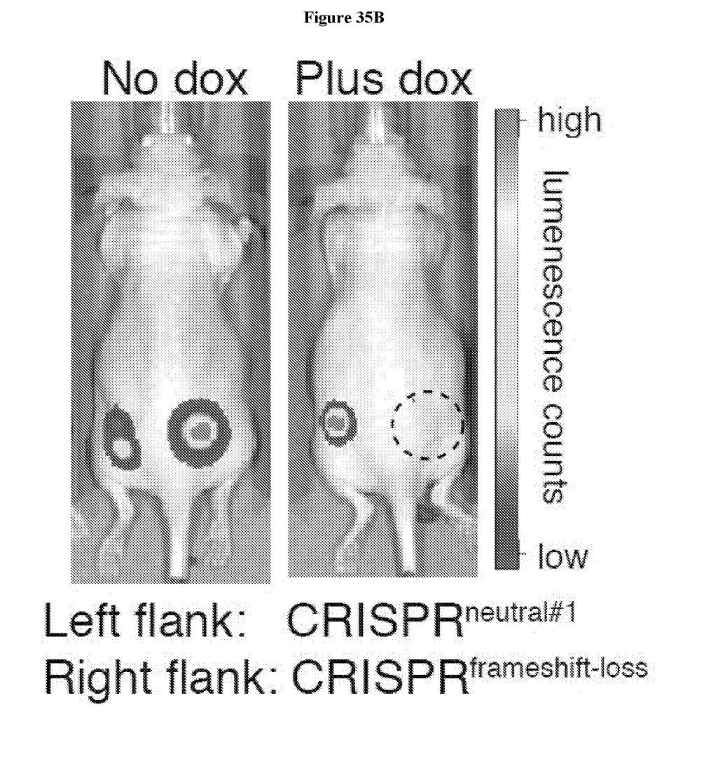

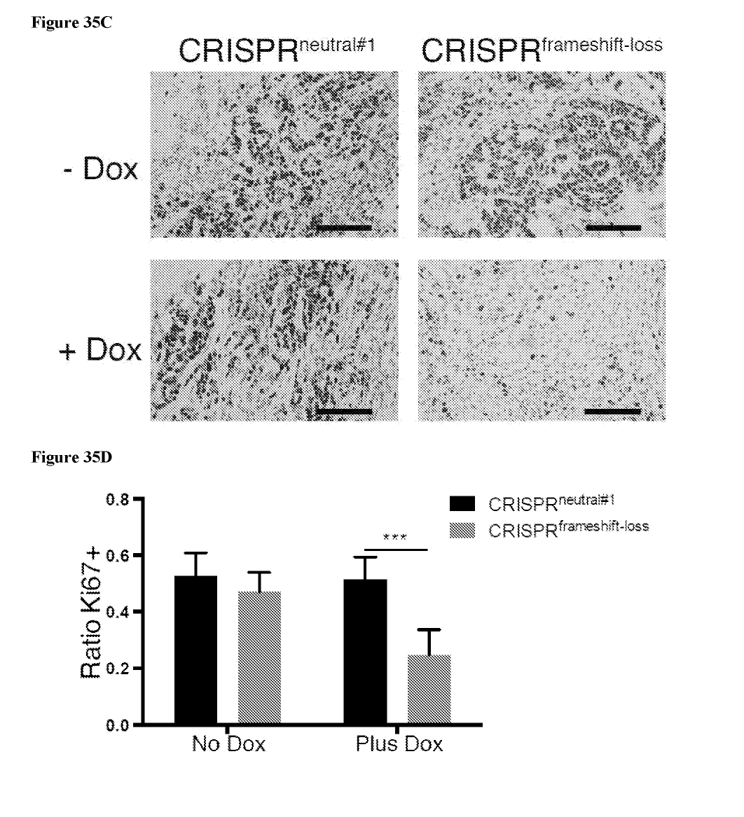

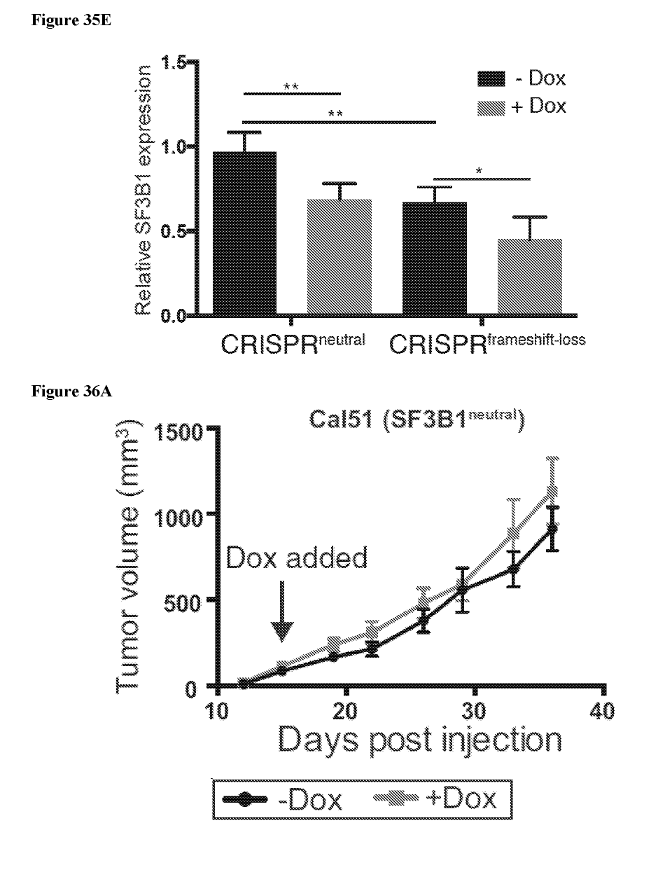

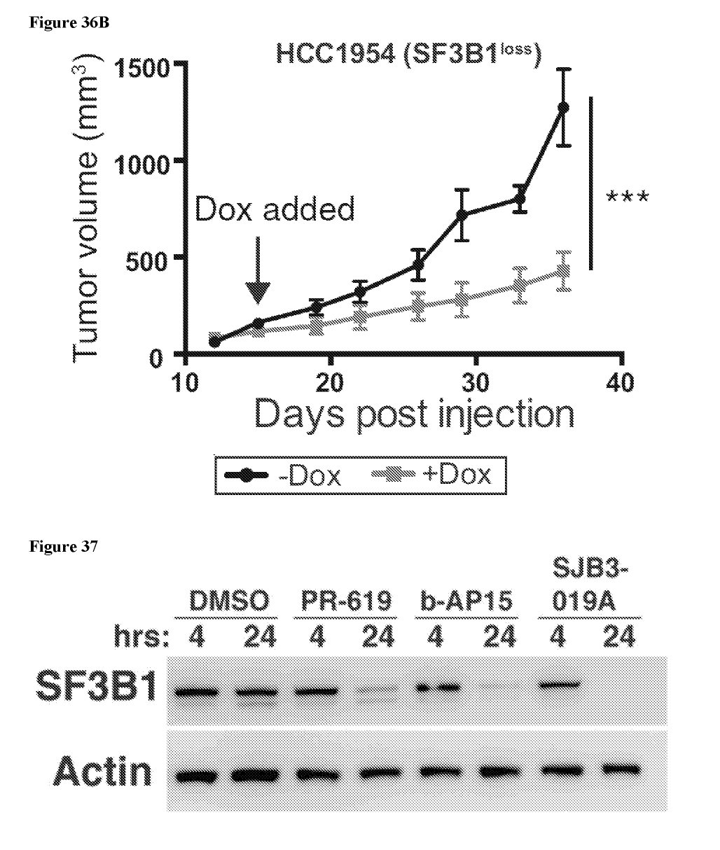

To test the effects of SF3B1 suppression in vivo, xenografts were generated using luciferase-labeled cell lines from the CRISPR^{frameshift-loss} and CRISPR^{neutral#1} cells containing TR-shSF3B1#3. Animals were placed on doxycycline upon detection of palpable tumors. CRISPR^{frameshift-loss} and CRISPR^{neutral#1} cells generated tumors of similar volume in the absence of doxycycline (FIG. 34; p=0.7, repeated measures ANOVA). However, suppression of SF3B1 reduced the growth of tumor (FIGS. 35A, 35B,35E) and number of proliferative Ki67+ cells (FIGS. 35C, 35D) in xenografts from CRISPR^{frameshift-loss}cells but not CRISPR^{neutral#1} cells (p=0.001 for both assays). Similarly, reduced tumor growth was observed in naturally occurring SF3B1^{loss} HCC1954 xenografts (FIG. 36B) and not in SF3B1^{neutral} Cal51 xenografts (FIG. 36A). Therefore, SF3B1 suppression specifically inhibited the progression of tumors with SF3B1 copy loss.

### Example 11

### Methods

### Analysis of Genome-Wide Copy-Number Associated Cancer Dependencies

Gene-level relative copy-numbers were downloaded from the CCLE portal (http://www.broadinstitute.org/ccle, data version Apr. 6, 2012). Gene-level dependencies were obtained for 214 cell lines from Project Achilles (version 2.4.3). ATARiS gene dependency scores were used to estimate the effect of shRNA-induced gene suppression on cell viability (Shao et al., 2013). Pearson correlation coefficients and associated p-values were calculated for the association of viability after suppression of each gene with the copy number of all genes. P-values were corrected for multiple hypotheses using the Benjamini-Hochberg method (Benjamini and Hochberg, 1995). Associations between copy-numbers of every gene in the genome and dependencies of every gene with ATARiS scores were considered. Large copy-number events affecting many neighboring genes often generated identical significant copy-number:gene dependency associations for copy-numbers associated with multiple genes. These were considered to reflect a single gene whose copy-number was responsible for the association. When the gene dependency reflected the same gene contained within the copy-number altered region, that gene was nominated as the source of the association. Likewise, if a gene dependency reflected a paralog of a gene within the copy-number altered region, its paralog was nominated.

### Classification of Length and Amplitude for Copy Number Alterations

For relative log2 normalized copy number data analyzed from 10,570 tumors from TCGA, the following thresholds were used for copy number classification: homozygous loss log2 values<−1.2, hemizygous loss log2<=−0.35. For cancer cell lines used in functional studies: copy-loss cells had log2 copy number<=−0.35, and copy neutral cells had log2 copy number>−0.2 and <0.2.

### Analysis of Gene Expression Across Normal Tissues

RNA sequencing data were downloaded from the GTEX database (http://www.gtexportal.org/home/). For every gene in the genome, the expression variance across all samples were calculated and the variance was ranked among the 20 genes with the most similar average expression level. These ranks served as a nearest-neighbors normalized measure of expression variance.

### CYCLOPS Analysis

The significance of differences in ATARiS scores between copy-neutral and loss lines were determined for every gene by comparing the observed data to data representing random permutations of copy-number class labels, each maintaining the number of cell lines and lineage distribution in each class. Copynumber classes were assigned as copy-loss for cells with log2 relative copy number ratios<=−0.35 and copy-neutral otherwise.

### Generation of Heterozygous SF3B1loss Cells by CRISPR-Cas9

For CRISPR^{frameshift-loss} cells, short guide RNAs targeting the first constitutively expressed coding exon of SF3B1 (exon 2) were designed with the aid of a web-based application (http://crispr.mit.edu/). Sense and anti-sense oligonucelotides were annealed and cloned into Bbsl site of pX458 (Addgene) and verified by Sanger sequencing. Single GFP+ cells were sorted by FACS and plated at low density for single cell cloning. CRISPR^{neutral#1} cells were processed identically, but did not have inactivating SF3B1 mutations. Oligonucleotide sequences for CRISPR^{frameshift-loss}were as follows: 5′-CACCGCATAATAACCTGTAGAATCG (forward), 5′-AAACCGATTCTACAGGTTATTATGC (reverse). pX458 was transfected with LipoD293 (SignaGen) into the diploid breast cancer cell line, Cal51. 19 monoclonal cell lines were genotyped for Cas-9 induced mutations by Sanger sequencing cloned PCR products. All monocolonal lines had either no mutations or harbored biallelic mutations in SF3B1. The genotypes of the Cal51 CRISPR cell lines used from this method of generation were: SF3B1delT36/delT36 (CRISPR^{neutral#1}) and SF3B1delT36/A23fsX20 (CRISPR^{frameshift-loss}). Copy number profiles from the two lines were characterized by SNP array. No SCNAs were detected as a result of single cell cloning (data not shown).

For CRISPR^{copy-loss} cells, a Cas9 construct co-expressing two sgRNAs and GFP was used to delete a 57 kb region encoding SF3B1. The guide RNA targeting the 5′ upstream of SF3B1 used a mismatch from a heterozygous SNP (rs3849362) in Cal51 to bias towards mono-allelic deletion of SF3B1. Single GFP+ cells were plated as described above and expanded. One of these was validated by PCR to harbor a 57 kb deletion encoding SF3B1. This was designated “CRISPR^{copy-loss}for subsequent experiments. Another one of these was found by PCR not to harbor this deletion and was designated as the control cell line for subsequent experiments (“CRISPR^{neutral#2}”).

Oligonucleotides for CRISPR^{copy-loss} cells were cloned in a similar fashion as CRISPR^{frameshift-loss} in pX458 (with BbsI overhangs). The sequences are as follows: For the 5′ guide targeting SNP, 5′-CACCGCGCATTATAGATTATGGCCC (forward) and 5′-AAACGGGCCATAATCTATAATGCGC (reverse). For the 3′ targeting guide: 5′-CACCGCGGAGTTTCATCCGTTACAC (forward), 5′-AAACGTGTAACGGATGAAACTCCGC (reverse) were used.

### Tissue Culture

Human cancer cell lines were maintained in RPMI-1640 supplemented with 10% fetal bovine serum and 1% penicillin and streptomycin and were assayed to be free of mycoplasma. Non-transformed MCF10A and HMEC cells were cultured in Mammary Epithelial Growth Medium (CC-3150, Lonza). For cells expressing tetracycline-regulated shRNAs, tetracycline-approved fetal bovine serum (Clonetech) was used.

shRNAs Targeting SF3B1

Lentiviral expression constructs for shRNA-mediated suppression of SF3B1 were obtained through the RNAi-consortium (http://www.broadinstitute.org/rnai/public/). The clone ID's and names used in our studies are as follows:

| shSF3B1 | Hairpin sequence |
|---|---|
| #2 | 5′-CCGG-CGCTATTGATTGATGAAGATT- |
| (TRCN0000320576) | CTCGAG-AATCTTCATCAATCAATAGCG- |
| | TTTTTG-3′ (SEQ ID NO: 20) |
| #3 | 5′-CCGG-CAACTCCTTATGGTATCGAAT- |
| (TRCN0000320566) | CTCGAG-ATTCGATACCATAAGGAGTTG- |
| | TTTTTG-3′ (SEQ ID NO: 21) |
| #4 | 5′-CCGG-TGCTTTGATTTGGTGATGTAA- |
| (TRCN0000350273) | CTCGAG-TTACATCACCAAATCAAAGCA- |
| | TTTTTG-3′ (SEQ ID NO: 22) |
| #5 | 5′-CCGG-CCTCGATTCTACAGGTTATTA- |
| (TRCN0000320636) | CTCGAG-TAATAACCTGTAGAATCGAGG- |
| | TTTTTG-3′ (SEQ ID NO: 23) |

Generation of Inducible SF3B1 shRNA Expression System

Sense and anti-sense oligonucleotides were annealed and cloned into the AgeI and EcoRI restriction sites of the pLKO-Tet-puro vector (Addgene, plasmid #21915). The oligonucleotide sequences were:

| shRNA | Sequence |
|---|---|
| LacZ (sense) | CCGGTGTTCGCATTATCCGAACCATCTCGAGATGG |
| | TTCGGATAATGCGAACATTTTTG (SEQ ID NO: |
| | 10) |
| LacZ (anti- | AATTCAAAAATGTTCGCATTATCCGAACCATCTCG |
| sense) | AGATGGTTCGGATAATGCGAACA (SEQ ID NO: |
| | 11) |
| TR-shSF3B1#3 | CCGGCAACTCCTTATGGTATCGAATCTCGAGATTC |
| (sense) | GATACCATAAGGAGTTGTTTTTG (SEQ ID NO: |
| | 12) |
| TR-shSF3B1#3 | AATTCAAAAACAACTCCTTATGGTATCGAATCTCG |
| (anti-sense) | AGATTCGATACCATAAGGAGTTG (SEQ ID NO: |
| | 13) |
| TR-shSF3B1#5 | CCGGCCTCGATTCTACAGGTTATTACTCGAGTAAT |
| (sense) | AACCTGTAGAATCGAGGTTTTTG (SEQ ID NO: |
| | 14) |
| TR-shSF3B1#5 | AATTCAAAAACCTCGATTCTACAGGTTATTACTCG |
| (anti-sense) | AGTAATAACCTGTAGAATCGAGG (SEQ ID NO: |
| | 15) |

### Cellular Growth Assays

Cells were plated in 96 well plates at 1000 cells per well. Cell number was inferred by ATPdependent luminescence by Cell Titer Glo (Promega) and normalized to the relative luminescence on the day of plating. For short-term lentiviral infections, cells were infected 24 hours prior to plating.

### GFP Competition Assays

Oligonucleotides encoding LacZ or SF3B1 shRNA#4 hairpin sequences were annealed and cloned into the pLK0.1 derivative vector TRC047 (pLKO.3pgw) and verified by Sanger sequencing. Cells were infected with serial dilutions of virus to achieve ˜50% GFP-positive cells. Cells with approximately equivalent ratios of GFP-positive -and negative cells were assayed by flow cytometry 3 days post infection and at subsequent time-points. The fold change in GFP+ cells was normalized to the percentage present 3 days after infection. For competition assays re-introducing exogenous SF3B1, expression of a human codon-optimized SF3B1 by lentivirus was utilized. Cells were infected as described above and treated with doxycycline two days after infection.

### Propidium Iodide Cell Viability Assays

Cells were treated with either short-term lentiviral infection or tetracycline-inducible SF3B1 shRNAs. After treatment, cells were trypsinized and pelleted including any cells in suspension. Cells were resuspended in propidium iodide viability staining solution (1× PBS, 1% BSA, 2.5 ug/mL propidium iodide) and quantified by live-cell flow cytometry. The change in viability was normalized to the percent of viable cells quantified on the first day of the assay.

### Determination of Cell Cycle Distribution by Propidium Iodide

Cells were trypsinized, washed and fixed with ice-cold 70% ethanol for a minimum of 15 minutes at 4 C. Cells were incubated in propidium iodide cell cycle staining solution (1× PBS, 1% BSA, 50 ug/mL propidium iodide, 100 ug/mL RNAse A) for 15 minutes and analyzed by flow cytometry. Debris and aggregates were gated out and cell cycle stage was quantified using Modfit (Varity Software House).

### Annexin-V Apoptosis Assays

Cellular apoptosis was quantified by live-cell flow cytometry using Alexa-Fluor 488 conjugated Annexin-V (Life Technologies) and propidium iodide. Cells were incubated in Annexin binding buffer containing propidium iodide (10 mM Hepes, 140 mM NaCl, 2.5 mM CaCl2, 2.5 ug/mL propdium iodide) for 15 min, washed and resuspended in FACS buffer (1× PBS, 1% BSA and 50 mM EDTA). Determination of the stage of apoptosis by gating was as follows: viable cells (Annexin-V−/PI−), early apoptosis (Annexin-V+/PI−), late apoptosis (Annexin-V+/PI+), and dead cells (Annexin-V−/PI+).

### Glycerol Gradient Sedimentation

Glycerol gradient sedimentation was performed as previously described (Klaus Hartmuth, 2012) with slight modifications for use with whole-cell lysates. Briefly, 10-30% glycerol gradients were formed by layering 10% glycerol gradient buffer (20 mM Hepes-KOH (pH 7.9), 150 mM NaCl, 1.5 mM MgCl2 10% glycerol) on top of a 30% glycerol buffer with identical salt concentrations. Gradients were formed using a Gradient Station (Biocomp Instruments). Cells were lysed in “IP lysis buffer” (50 mM Tris, 150 mM NaCl and 1% Triton X-100). 400 uL containing 1-3 mg of crude lysate was loaded per gradient in SW55 centrifuge tubes and spun at 55,000 RPM for 3.5 hours at 4 C. 200 uL fractions were collected by manually pipetting from the top of the gradient. Recombinant proteins of known mass were run in parallel gradients as controls.

### Gel Filtration Chromatography

Sephacryl S-500 (17-0613-05, GE Healthcare) columns were packed into a 50×1.5 cm column and equilibrated with column buffer (10 mM Tris, 60 mM KCl, 25 mM EDTA, 1% Triton X-100 and 0.1% sodium azide). Whole-cell lysates were collected in IP lysis buffer as described above and incubated with 0.5 mM ATP, 3.2 mM MgCl2 and 20 mM creatine phosphate (di-Tris salt) for 20 min at 30 C to dissociate multi-snRNP spliceosomal complexes. 2 mL of lysate containing 5 mg of protein was loaded on columns and 90 1.5 mL fractions were collected overnight at 4 C.

### Western Blotting

For denaturing protein immunoblots, cells were washed in ice cold PBS and lysed in 1× RIPA buffer (10 mM Tris-Cl Ph 8.0, 1 mM EDTA, 1% Triton X-100, 0.1% SDS and 140 mM NaCl) supplemented with lx protease and phosphatase inhibitor cocktail (PI-290, Boston Bioproducts). Lysates were sonicated in a bioruptor (Diagenode) for 5 minutes (medium intensity) and cleared by centrifugation at 15000×g for 15 min at 4 C. Proteins were electrophoresed on polyacrylamide gradient gels (Life Technologies) and detected by chemiluminescence. For native western blotting, cells were washed in ice cold PBS and lysed in 1× sonication buffer (10% Glycerol, 25 mM HEPES pH 7.4, 10 mM MgCl2) supplemented with protease and phosphatase inhibitors. Coomassie blue native PAGE western blots were run according the manufacturer's instructions (Life Technologies).

### Immunoprecipitation

Immunoprecipitations were performed with pooled glycerol gradient fractions. The Fc region of mouse anti-SF3B1 (Medical and Biological Laboratories, D221-3) was directionally cross-linked to protein G Dynabeads (Life Technologies) using 20 mM dimethyl pimelimidate (DMP). IgG isotype controls were cross-linked and processed identically. Proteins were eluted with elution buffer (15% glycerol, 1% SDS, 50 mM tris-HCl, 150 mM NaCl pH 8.8) at 80 C and subjected to western blot analysis.

### Quantitative and Reverse Transcription PCR

RNA was extracted using the RNeasy extraction kit (Qiagen) and subjected to on-column DNase treatment. cDNA was synthesized with the Superscript II Reverse Transcriptase kit (Life Technologies) with no reverse transcriptase samples serving as negative controls. Gene expression was quantified by Power Sybr Green Master Mix (Applied Biosystems). Primers for all genes were determined to be equally efficient over 5 serial two-fold dilutions. Gene expression values were normalized to ACTB and the fold change calculated by the ΔΔCt method. For quantification of the U2 snRNA, the above method was used except total cellular RNA was extracted with Trizol (Life Technologies). SF3B1 qPCR primer sequences: (forward) 5′-ccaaagattgcagaccggga-3′ (SEQ ID NO: 24), (reverse) 5′-tcaggggttttccctccatc-3′ (SEQ ID NO: 25). These primers detect all three splicing variants of SF3B1 (SEQ ID NOs: 2-4).

### Library Preparation and RNA-Sequencing

Total RNA was extracted with the RNeasy mini extraction kit (Qiagen) and treated by on-column DNAse digestion. RNA quality was determined with a bioanalyzer (Agilent) and samples with RIN values>7 were used for sequencing. mRNA were enriched with the NEBNext Poly(A) mRNA Magnetic Isolation Module (New England BioLabs, #E7490S). Library preparations for paired end sequencing were performed using the NEBNext Ultra Directional RNA Library Prep Kit for Illumina (New England BioLabs, #E7420S) according to manufacturer's specifications. Samples were pooled and 75 bp paired reads were generated using a NextSeq 500 sequencer (Illumina). Approximately 50 million reads per sample were generated.

### RNA Sequencing Analysis

FASTQ files were aligned using TOPHAT v1.4 with parameters “—mateinner-dist 300 —mate-std-dev 500 —no-sort-bam —no-convert-bam -p 4”. juncBase was used to identify read counts at splice junctions. The spliced in/spliced out counts at each junction were used to create an estimate of the risk of retaining an intron for each cell line. The distribution of this statistic was calculated for each cell line in each condition (with and without SF3B1 suppression) using a beta binomial distribution in which spliced in and spliced out read counts were the a and 13 terms, respectively. The distribution over relative risk of intron retention upon SF3B1 suppression was calculated as follows. For every quintile of the beta binomial distribution for the SF3B1 suppressed state, the ratio of the two beta-binomial distributions for each cell line was calculated. The posterior distributions over the relative risk of intron retention were combined for cell lines of the same genotype (i.e. SF3B1^{neutral} or SF3B1^{loss}) by obtaining the product of their distributions. P-values were obtained by calculating the overlap in the distributions over the relative risk of intron retention in each of the genotypes.

SF3B1 Gene Expression Analysis from TCGA and CCLE Datasets

Relative copy number and Affymetrix expression data for SF3B1 were downloaded from the CCLE portal from the Broad Institute (presently at www dot broadinstitute dot org forward slash ccle forward slash home). TCGA breast adenocarcinoma data were downloaded from the cBioPortal (presently at www dot cbioportal dot org forward slash public-portal forward slash index dot do). For both datasets, samples lacking either gene expression or copy-number were removed. As described above, copy-loss was defined as samples with log2 normalized relative copy number of <−0.35, copy gain was defined as >=0.3.

Nuclear Speckle Quantification by SC-35 Immunofluorescence with CellProfiler Image Analysis

Cells were plated on 35 mm glass bottom dishes with #1.5 cover glass (D35-14-1.5-N, In Vitro Scientific). Cells were fixed and stained with anti-SC-35 antibody (S4045, Sigma-Aldrich) at 1:1000 dilution and detected with Alexafluor488 secondary antibody at 1:1000 (Life Technologies). Nuclei were counterstained with Hoescht dye. Monochromatic images were captured under identical conditions and pseudo-colored using Photoshop. A custom image analysis pipeline was empirically adapted from a preexisting pipeline designed for detecting H2AX foci using CellProfiler (Kamentsky et al., 2011). Measurements of nuclear speckles were generated from at least 15 random microscopic fields. A minimum of 100 nuclei identified by CellProfiler were used for quantitation per treatment.

Correlation Analysis of Copy-Loss of SF3b Genes with Cell Dependencies Upon Suppression of Other SF3b Complex Genes

Relative copy number and ATARiS gene dependency scores were determined after knockdown of each SF3b complex member across the same 179 cell lines used in the CYCLOPS analysis. Linear regression analysis was performed for copy number of each SF3b complex gene with knockdown of every SF3b component. One-sided p-values were calculated for association of sensitivity to suppression with gene loss for all intra-SF3b complex comparisons. Samples were excluded if they harbored co-deletion of the two genes used to generate the correlation.

### Generation of Xenografts and Growth Assessment

All animal husbandry was done with the approval of the Dana-Farber Cancer Institute IACUC. 1×10⁶ CRISPR^{neutral#1} or CRISPR^{frameshift-loss} cells expressing TR-shSF3B1 #3 were subcutaneously injected into opposing flanks of nude mice (Foxn1 nu/nu, Harlan). Animals were randomized to control group or doxycycline treatment after detection of a palpable tumor on either flank. Mice in the doxycycline treatment arm were continuously fed a doxycycline diet (2,000 ppm). Mice were sacrificed at the end of the experiment, or when endpoints were reached based on failure to thrive according to IACUC recommendation. Repeated measures two-way ANOVA was used to assess significance.

A custom image analysis pipeline was used to systematically quantify Ki67+ cells from tumor xenografts using CellProfilier. A minimum of 3 tumors per group, totaling at least 2,440 nuclei per tumor, was used to quantify the ratio of Ki67+ cells. At least 5 individual and random microscopic images from each tumor were analyzed.

### Sequence Listing

SEQ ID NO: 1—human SF3B1 genomic sequence (NG_032903.2 nucleotide 4955..48074)
SEQ ID NO: 2—human SF3B1 mRNA sequence, transcript variant 1 (NM_012433.3)
SEQ ID NO: 3—human SF3B1 mRNA sequence, transcript variant 2 (NM_001005526.2)
SEQ ID NO: 4—human SF3B1 mRNA sequence, transcript variant 3 (NM_001308824.1)
SEQ ID NO: 5—human SF3B1 protein sequence, isoform 1 (NP_036565.2)
SEQ ID NO: 6—human SF3B1 protein sequence, isoform 2 (NP_001005526.1)
SEQ ID NO: 7—human SF3B1 protein sequence, isoform 3 (NP_001295753.1)
SEQ ID NO: 8—target sequence of TR-shSF3B1#3 on human SF3B1 mRNA

CAACTCCTTATGGTATCGAATCT

SEQ ID NO: 9—target sequence of TR-shSF3B1#5 on human SF3B1 mRNA

CCTCGATTCTACAGGTTATTA

SEQ ID NOs: 10-15—see example 11
SEQ ID NO: 16—target sequence of shSF3B1 #2 on human SF3B1 mRNA

CGCTATTGATTGATGAAGATT

SEQ ID NO: 17—target sequence of shSF3B1 #3 on human SF3B1 mRNA

CAACTCCTTATGGTATCGAAT

SEQ ID NO: 18—target sequence of shSF3B1 #4 on human SF3B1 mRNA

TGCTTTGATTTGGTGATGTAA

SEQ ID NO: 19—target sequence of shSF3B1 #5 on human SF3B1 mRNA

CCTCGATTCTACAGGTTATTA

SEQ ID NOs 20-23—see example 11
SEQ ID NO: 24—forward primer for amplifying human SF3B1 mRNA or cDNA

ccaaagattgcagaccggga

SEQ ID NO: 25—reverse primer for amplifying human SF3B1 mRNA or cDNA

tcaggggttttccctccatc

1. A method for determining the likelihood that a subject with cancer responds to an SF3B1 suppression treatment, comprising measuring the copy number of SF3B1 in a sample comprising cells from the subject, wherein the likelihood is increased if the copy number of SF3B1 in the sample from the subject is smaller than the ploidy of the cells in the sample.

2.-15. (canceled)

16. A method for determining the likelihood that a subject with cancer responds to an SF3B1 suppression treatment, comprising
a. measuring expression level of SF3B1 in a sample from the subject; and
b. comparing the measured expression level of SF3B1 in the sample from the subject to the expression level of SF3B1 in a control sample,
wherein the likelihood that a subject with cancer responds to an SF3B1 suppression treatment is increased if the expression level of SF3B1 in the sample from the subject is lower than the expression level of SF3B1 in the control sample.

17.-45. (canceled)

45. A method for treating a subject with cancer, comprising providing an SF3B1 suppression treatment, thereby treating the cancer in the subject.

46. A method for treating a subject with cancer, comprising
a. measuring the copy number of SF3B1 in a sample comprising cells from the subject; and
b. providing an SF3B1 suppression treatment if the copy number of SF3B1 in the sample from the subject is smaller than the ploidy of the cells in the sample,
thereby treating the cancer in the subject.

47. The method of claim 46, wherein the cancer is selected from the group consisting of breast cancer, hematopoietic cancer, bladder cancer and kidney cancer.

48. The method of claim 47, wherein the cancer is selected from the group consisting of breast cancer and hematopoietic cancer.

49. The method of claim 46, wherein the cancer is selected from the group consisting of acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, chronic myelogenous leukemia, colon adenocarcinoma, esophageal carcinoma, glioblastoma multiforme, head and neck squamous cell carcinoma, chromophobe renal cell carcinoma, renal clear cell carcinoma, renal papillary cell carcinoma, hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma.

50. The method of claim 46, wherein the sample comprises a cancerous lesion.

51. The method of claim 46, wherein the sample comprises circulating tumor cells.

52. The method of claim 46, wherein measuring the copy number of SF3B1 comprises comparative genomic hybridization (CGH).

53. The method of claim 46, wherein measuring the copy number of SF3B1 comprises fluorescence in situ hybridization (FISH).

54. The method of claim 46, wherein measuring the copy number of SF3B1 comprises amplifying a genomic sequence comprising at least 20 nucleotides of SF3B1.

55. The method of claim 46, wherein measuring the copy number of SF3B1 comprises DNA sequencing.

56. The method of claim 55, wherein DNA sequencing comprises whole-genome sequencing.

57. The method of claim 55, wherein DNA sequencing comprises whole-exome sequencing.

58. The method of claim 46, wherein the copy number of SF3B1 in the sample is an average copy number if the sample is heterogeneous.

59. The method of claim 58, wherein the average copy number of SF3B1 in the sample from the subject is smaller than the ploidy of the cells in the sample by at least 25%.

60. A method for treating a subject with cancer, comprising
a. measuring expression level of SF3B1 in a sample from the subject;
b. comparing the measured expression level of SF3B1 in the sample from the subject to the expression level of SF3B1 in a control sample; and
c. providing an SF3B1 suppression treatment if the expression level of SF3B1 in the sample from the subject is lower than the expression level of SF3B1 in the control sample,
thereby treating the cancer in the subject.

61.-86. (canceled)

87. A kit comprising:
a. a reagent for reverse transcription of an RNA molecule;
b. two or more primers, wherein a first primer comprises a polynucleotide comprising SEQ ID NO: 24, and a second primer comprises a polynucleotide comprising SEQ ID NO: 25; and
c. a reagent for amplification of a DNA sequence.

88. A kit comprising an antibody that is capable of binding SF3B1 and a reagent for the detection of the antibody.
